(19) United States
(12) Patent Application Publication
(10) Pub. No.: US 2021/0002650 A1
(43) Pub. Date: Jan. 7, 2021

(54) COMPOSITIONS AND METHODS FOR TREATING ALZHEIMER'S DISEASE

(71) Applicant: University of Virginia Patent Foundation, Charlottesville, VA (US)

(72) Inventors: George S. Bloom, Charlottesville (US); Erin J. Kodis, Ardmore, PA (US)

(73) Assignee: University of Virginia Patent Foundation, Charlottesville, VA (US)

(21) Appl. No.: 16/921,366

(22) Filed: Jul. 6, 2020

Related U.S. Application Data
(60) Provisional application No. 62/870,412, filed on Jul. 3, 2019.

(51) Int. Cl. C12N 15/113; A61K 31/439; A61K 31/4402; A61K 31/135; A61P 25/28

(52) U.S. Cl. CPC C12N 15/1138; A61K 31/439; A61P 25/28; A61K 31/135; A61K 31/4402

(57) ABSTRACT

Provided are methods for treating Alzheimer's disease (AD), which in some embodiments can include administering to a subject in need thereof a composition that includes an inhibitor of an Aβ oligomer (AβO) biological activity. Also provided are methods for inhibiting development and/or progression of at least one symptom associated with AD, methods for inhibiting neuronal cell cycle re-entry (CRR), methods for inhibiting Aβ oligomer (AβO) biological activity, methods for inhibiting Aβ oligomer (AβO)-stimulated activation of calcium-calmodulin-dependent protein kinase II (CaMKII) biological activity, and methods for inhibiting calcium influx-induced excitotoxic neuronal death. In some embodiments, the inhibitor of the AβO biological activity or the inhibitor of N-methyl-D-aspartate receptor (NMDAR) signaling includes a small molecule inhibitor, an inhibitory nucleic acid, a calcium chelator, or any combination thereof. Also provided are methods for treating subjects who are pre-symptomatic for AD with inhibitors of AβO and/or NMDAR biological activities.

### CROSS REFERENCE TO RELATED APPLICATION

The presently disclosed subject matter claims the benefit of U.S. Provisional Patent Application Ser. No. 62/870,412, filed Jul. 3, 2019, the disclosure of which incorporated herein by reference in its entirety.

### GOVERNMENT INTEREST

This presently disclosed subject matter was made with government support under Grant No. AG051085, awarded by the National Institutes of Health. The government has certain rights in the presently disclosed subject matter.

### BACKGROUND

Alzheimer's disease (AD) is a devastating neurological disorder characterized by memory loss and cognitive decline. These behavioral symptoms are caused at the cellular level by synaptic dysfunction and loss, and neuron death, and at the molecular level by toxic forms of amyloid-β (AD) and tau that work coordinately to damage synapses [1-3] reduce insulin signaling [4], impair axonal transport [5], and kill neurons [6-8]. While poorly soluble, fibrillar forms of Aβ and tau that respectively are found in plaques and tangles are histopathological hallmarks of AD, soluble oligomeric forms of Aβ and tau are now recognized as being far more toxic [9-11]. It follows naturally that efforts to prevent and treat AD will benefit from advances in our still primitive understanding of the pathogenic signaling mechanisms that underlie the breakdown of normal neuronal homeostasis caused by toxic oligomers of Aβ and tau.

As much as 90% of neuron death in AD may follow ectopic re-entry of neurons into the cell cycle [12,13]. Whereas fully differentiated, healthy neurons are permanently post-mitotic, affected neurons in AD and other neurodegenerative disorders often express molecular markers of the G1 and S-phase stages of the cell cycle [14-17]. Instead of dividing, however, these neurons apparently die after a delay of up to hundreds of days following cell cycle re-entry (CCR) [18]. Previous research defined a CCR signaling network in AD, whereby Aβ oligomers (AβOs) induce activation of multiple protein kinases that catalyze site-specific tau phosphorylation, and a positive feedback loop between phospho-tau and the multi-subunit protein kinase complex, mTORC1, that leads to mTORC1 dysregulation and drives neurons from the G0 phase of the cell cycle into the G1 phase [19,20].

Besides triggering CCR and neuron death, AβOs cause excitotoxicity by stimulating excess calcium influx into neurons [21-23]. This disruption of normal calcium homeostasis affects numerous signaling pathways, and can damage and destroy synapses, and lead to abrupt neuron death [24]. A major contributor to this pathological process is the N-methyl-D-aspartate receptor (NMDAR), which permits toxic levels of calcium to enter neurons exposed to AβOs [3,25]. Interestingly, one of the few FDA-approved treatments for AD is memantine, which works by blocking excess calcium entry into neurons via NMDAR [26].

There is a long felt need in the art for compositions and methods useful for blocking the pathways involved in AD and for treating pre-symptomatic and symptomatic AD. The presently disclosed subject matter satisfies these needs.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

It is disclosed herein that memantine is useful for treating or preventing pre-symptomatic AD.

In some embodiments, a compound of the presently disclosed subject matter is useful for treating or preventing at least one symptom associated with AD. In some embodiments, a compound of the presently disclosed subject matter is useful for treating or preventing two or more symptoms of AD.

In some embodiments, administering a compound of the presently disclosed subject matter to a subject in need thereof slows AD progression.

In some embodiments, administering a compound of the presently disclosed subject matter to a subject in need thereof prevents AD progression.

In some embodiments, a compound of the presently disclosed subject matter can be administered when the subject is asymptomatic for AD.

In some embodiments, a compound of the presently disclosed subject matter can be administered when the subject has symptoms of AD.

NAMENDA® (memantine hydrochloride) is an orally active NMDA receptor antagonist. The molecular formula is C₁₂H₂₁N.HCl and the molecular weight is 215.76 dalton (Da). The CAS number is 41100-52-1. Memantine HCl occurs as a fine white to off-white powder and is soluble in water. The chemical name for memantine hydrochloride is 1-amino-3,5-dimethyladamantane hydrochloride. Synonyms for the chemical name include: 3,5-dimethyl-1-adamantanamine hydrochloride, 3,5-dimethylamantadine hydrochloride, and 3,5-dimethyltricyclo[3.3.1.1^{3,7}]decan-1-amine hydrochloride. The chemical has the following structural formula:

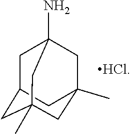

The present application provides compositions and methods to inhibit or prevent neuronal CCR.

In some embodiments, the present applications provides compositions and methods for pharmacologically inhibiting NMDAR activity. In some embodiments, inhibiting NMDAR activity inhibits neuronal CCR. In some embodiments, the inhibitor of NMDAR activity is memantine. In some embodiments, the inhibitor of NMDAR activity is an shRNA.

In some embodiments, the present application provides compositions and methods for regulating AβO to treat AD.

In some embodiments, the present application provides composition and methods useful for inhibiting AβO-stimulated activation of calcium-calmodulin-dependent protein kinase II (CaMKII). In some embodiments, the method inhibits neuronal CCR. In some embodiments, the composition comprises a compound to inhibit AβO-stimulated activation of CaMKII. In some embodiments, the composition comprises a compound to inhibit AβO activity. In some embodiments, the compound is memantine.

In some embodiments, the present application provides compositions and methods for inhibiting excitotoxic neuron death caused by excess calcium influx. In some embodiments, the excess calcium influx is initiated by AβOs.

In some embodiments, shRNAs are useful for inhibiting or preventing CCR. In some embodiments, the shRNA is an antisense RNA. In some embodiments, the shRNA is directed to NR1, the constitutive subunit of NMDAR.

In some embodiments, a calcium chelator can be used to inhibit or prevent CCR.

In some embodiments, two or more of the treatments or compositions of the presently disclosed subject matter can be used as a combination therapy.

The presently disclosed subject matter also encompasses blocking other parts of the signaling pathways disclosed herein.

In some embodiments, a therapeutically effective amount of a compound of the presently disclosed subject matter is administered to a subject in need thereof. In some embodiments, a pharmaceutical composition comprising a compound of the presently disclosed subject matter is administered to the subject. One of ordinary skill in the art can determine the dosage to be administered based on the compound being administered and the age, sex, and health of the subject.

The presently disclosed subject matter encompasses the use of all types of inhibitors of the pathways described herein. The inhibitors include, but are not limited to, oligonucleotides, antisense oligonucleotide, nucleic acid, siRNA, shRNA, an antibody, antibody fragment, humanized antibody, monoclonal antibody, fragments thereof, aptamer, phylomer, protein, and small molecules such as drugs.

In some embodiments, the presently disclosed subject matter encompasses the use of siRNA silencing and pharmacological inhibition.

Thus, in some embodiments the presently disclosed subject matter relates to methods for treating Alzheimer's disease (AD). In some embodiments, the methods comprise, consist essentially of, or consist of administering to a subject in need thereof a composition comprising, consisting essentially of, or consisting of an effective amount of an inhibitor of an Aβ oligomer (AβO) biological activity.

In some embodiments, the presently disclosed subject matter also relates to methods for inhibiting development and/or progression of at least one symptom associated with Alzheimer's disease (AD). In some embodiments, the methods comprise, consist essentially of, or consist of administering to a subject in need thereof a composition comprising, consisting essentially of, or consisting of an effective amount of an inhibitor of an Aβ oligomer (AβO) biological activity.

In some embodiments, the presently disclosed subject matter also relates to methods for inhibiting neuronal cell cycle re-entry (CRR). In some embodiments, the methods comprise, consist essentially of, or consist of administering to a subject in need thereof a composition comprising, consisting essentially of, or consisting of an effective amount of an inhibitor of an Aβ oligomer (AβO) biological activity.

In some embodiments, the presently disclosed subject matter also relates to methods for inhibiting Aβ oligomer (AβO) biological activity. In some embodiments, the methods comprise, consist essentially of, or consist of administering to a subject in need thereof a composition comprising, consisting essentially of, or consisting of an effective amount of an inhibitor of N-methyl-D-aspartate receptor (NMDAR) signaling.

In some embodiments, the presently disclosed subject matter also relates to methods for inhibiting Aβ oligomer (AβO)-stimulated activation of calcium-calmodulin-dependent protein kinase II (CaMKII) biological activity. In some embodiments, the methods comprise, consist essentially of, or consist of administering to a subject in need thereof a composition comprising, consisting essentially of, or consisting of an effective amount of an inhibitor of an Aβ oligomer (AβO) biological activity.

In some embodiments, the presently disclosed subject matter also relates to methods for inhibiting calcium influx-induced excitotoxic neuronal death. In some embodiments, the methods comprise, consist essentially of, or consist of administering to a subject in need thereof a composition comprising, consisting essentially of, or consisting of an effective amount of an inhibitor of an Aβ oligomer (AβO) biological activity.

In some embodiments of the presently disclosed methods, the inhibitor of the Aβ oligomer (AβO) biological activity or the inhibitor of N-methyl-D-aspartate receptor (NMDAR) signaling comprises a small molecule inhibitor, an inhibitory nucleic acid, a calcium chelator, or any combination thereof.

In some embodiments, the small molecule inhibitor is selected from the group consisting of 3,5-dimethyladamantan-1-amine, (1S,9R)-1-methyl-16-azatetracyclo[7.6.1.02,7.010,15]hexadeca-2,4,6,10,12,14-hexaene (MK-801), (1S)-1-phenyl-2-pyridin-2-ylethanamine (lanicemine), ketamine, metabolic precursors thereof, biologically active metabolic products thereof, derivatives thereof, and pharmaceutically acceptable salts thereof, optionally wherein the derivative is nitromemantine, or any combination thereof.

In some embodiments, the inhibitory nucleic acid targets a nucleic acid encoding a component of an N-methyl-D-aspartate receptor (NMDAR), optionally wherein the component of the NMDAR is an NR1 gene product.

In some embodiments, the inhibitory nucleic acid targets a human NR1 gene product, optionally wherein the human NR1 gene product comprises any one of SEQ ID NOs: 1-12.

In some embodiments, the inhibitory nucleic acid targets a human NR2 gene product, optionally wherein the human NR2 gene product comprises any one of SEQ ID NOs: 19-25.

In some embodiments of the presently disclosed methods, the subject is pre-symptomatic for AD. In some embodiments, the inhibitor of an Aβ oligomer (AβO) biological activity comprises a small molecule inhibitor, an inhibitory nucleic acid, a calcium chelator, or any combination thereof. In some embodiments, the small molecule inhibitor is selected from the group consisting of 3,5-dimethyladamantan-1-amine, (1S,9R)-1-methyl-16-azatetracyclo[7.6.1.02,7.010,15]hexadeca-2,4,6,10,12,14-hexaene (MK-801), (1S)-1-phenyl-2-pyridin-2-ylethanamine (lanicemine), ketamine, metabolic precursors thereof, biologically active metabolic products thereof, derivatives thereof, and pharmaceutically acceptable salts thereof, optionally wherein the derivative is nitromemantine, or any combination thereof. In some embodiments, the inhibitory nucleic acid targets a nucleic acid encoding a component of an N-methyl-D-aspartate receptor (NMDAR), optionally wherein the component of the NMDAR is an NR1 gene product. In some embodiments, the inhibitory nucleic acid targets a human NR1 gene product, optionally wherein the human NR1 gene product comprises any one of SEQ ID NOs: 1-12. In some embodiments, the inhibitory nucleic acid targets a human NR2 gene product, optionally wherein the human NR2 gene product comprises any one of SEQ ID NOs: 19-25.

In some embodiments of the presently disclosed methods, the subject is APOE4-positive.

In some embodiments of the presently disclosed methods, the neuronal cell cycle re-entry (CRR), the Aβ oligomer (AβO) biological activity, the AβO-stimulated activation of calcium-calmodulin-dependent protein kinase II (CaMKII) biological activity, or the calcium influx-induced excitotoxic neuronal death is associated with development and/or progression of Alzheimer's disease (AD) in the subject.

Accordingly, it is an object of the presently disclosed subject matter to provide compositions and methods for modulating biological activities mediated by Aβ oligomers (AβOs), including those related to NMDAR and/or CaMKII signaling.

This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A and 1B. Intracellular calcium chelation by BAPTA-AM prevents AβO-induced neuronal CCR. FIG. 1A is a fluorescence micrograph of primary cortical neurons treated for 18 hours with AβOs with or without 10 μM BAPTA-AM to chelate intracellular calcium. CCR neurons were identified by their immunoreactivity with antibodies to both cyclin D1 (red; third panel from the left), which marks nuclei during G1 of the cell cycle, and NeuN (blue; second panel from the left), which marks nuclei in all neurons. Neuronal somatodendritic compartments were labeled with an antibody to MAP2 (green; left-most panel). A merge of the three panels is provided as the right-most panel. FIG. 1B is a bar graph of quantification of the immunofluorescence results and statistical analysis by one-way ANOVA. Error bars indicate s.e.m.

FIGS. 2A-2D. The NMDAR inhibitor, MK-801, blocks AβO-induced neuronal CCR and early activation of CaMKII. FIG. 2A is a fluorescence micrograph of primary cortical neurons treated overnight with AβOs with or without 10 μM MK-801. After 18 hours of exposure to AβOs, neurons were stained for MAP2 (left-most panel; green), NeuN (second panel from the left; blue), and Cyclin D1 (third panel from the left; red) to mark neurons that re-enter the cell cycle. A merge of the three panels is provided as the right-most panel. FIG. 2B is a bar graph of quantification of the immunofluorescence results and statistical analysis by one-way ANOVA. Error bars indicate s.e.m. FIG. 2C is a series of photographs of phospho-activated and total CaMKII levels monitored by western blotting of cortical neurons treated for the indicated times with AβOs, with or without 10 μM MK-801. FIG. 2D is a graph of quantification of phosphorylated CaMKII relative to total CaMKII at each time point in the presence (red squares) or absence (blue circles) of MK-801. Note that MK-801 prevented the transient rise in phospho-activation of CaMKII. Results are significant by two-way ANOVA when comparing the plus versus minus MK-801 data sets (p<0.005), and by Bonferroni post hoc analysis when comparing the 0 and 15-minute time points without MK-801 (p<0.01), and when comparing plus and minus MK-801 at the 15 minute time point (p<0.0001). Error bars indicate s.e.m.

FIGS. 3A-3C. Knockdown of the NR1 subunit of NMDAR prevents AβO-induced neuronal CCR. FIG. 3A is a fluorescence micrograph of primary cortical neurons transduced for 96 hours prior to AβO addition with lentivirus expressing shRNA to NR1, or as a control, with lentivirus comprising an empty expression vector. After 16-18 hours of AβO exposure, the cells were stained by triple immunofluorescence for NeuN (blue) and MAP2 (green) to mark neurons, or for cyclin D1 (red) to assess CCR. A merge of the NeuN, MAP2, and cyclin D1 panels for each treatment are shown at the bottom of FIG. 3A. FIG. 3B is a bar graph of quantification of the immunofluorescence results for each treatment with (gray bars) or without (white bars) AβOs. Quantification of the immunofluorescence results and statistical analysis by one-way ANOVA are presented. Error bars indicate s.e.m. FIG. 3C depicts the results of quantitative western blotting and shows a 30% knockdown of NR1. β-III tubulin was included as a loading control.

FIGS. 4A and 4B. Memantine blocks AβO-induced neuronal CCR. FIG. 4A is a fluorescence micrograph of primary cortical neurons treated with AβOs with or without pre-treatment with 10 μM memantine. Cells were then stained with antibodies to MAP2 (green), NeuN (blue), and cyclin D1 (red) to identify CCR neurons. A merge of each set of three panels is provided as the right-most panel. FIG. 4B is a bar graph of quantification of the immunofluorescence results and statistical analysis by one-way ANOVA. Error bars indicate s.e.m.

FIGS. 5A and 5B. Treatment of Tg2576 AD model mice with memantine prevents CCR in vivo. FIG. 5A is a fluorescence micrograph of brain sections of Tg2576 and wild type mice provided ad libitum access to drinking water with or without memantine from the time of weaning at 3 weeks until 2 months of age from each condition stained by triple immunofluorescence for the neuron-specific protein βIII-tubulin (green), cyclin D1 (red), and NeuN (aqua) to identify CCR positive neurons. A merge of each set of three panels is provided as the right-most panel. FIG. 5B is a bar graph of quantification of the immunofluorescence results and statistical analysis by one-way ANOVA. Error bars indicate s.e.m.

FIG. 6. AβO-induced calcium influx via NMDAR is necessary for neuronal CCR. Depicted are pathways by which AβO-induced excitotoxicity can contribute to many neuronal dysfunctions in AD, including synaptic deficits. As depicted, AβO-mediated calcium influx via NMDAR is also necessary for initiating CCR, and AβO-mediated synaptic dysfunctions involving NMDAR can initiate neuronal CCR directly, or alternatively, these pathways could diverge after AβO-mediated calcium influx, each contributing to neuron death independently. The two schemes depicted are not necessarily mutually exclusive.

FIGS. 7A and 7B: The selective α-amino-3-hydroxy-5-methyl-4-isoxazolepropionic acid (AMPA) receptor inhibitor, CNQX, does not inhibit CCR. FIG. 7A is a fluorescence micrograph of primary cortical neurons treated overnight with AβOs with or without 10 μM CNQX. After 18 hours of exposure to AβOs, neurons were stained for MAP2 (green), NeuN (blue), or cyclin D1 (red) to mark neurons that re-enter the cell cycle. A merge of each set of three panels is provided as the right-most panel. FIG. 7B is a bar graph of the quantification of the immunofluorescence results. Both untreated neurons minus AβOs versus plus AβOs, and CNQX treated neurons minus AβOs versus plus AβOs, showed significant increases in CCR (p<0.0001 by one-way ANOVA), indicating that AMPAR inhibition did not inhibit CCR from occurring.

FIGS. 8A and 8B: The endoplasmic reticulum inhibitors to IP₃ and ryanodine receptors do not block activation of CCR. FIG. 8A is a fluorescence micrograph of primary cortical neurons treated overnight with AβOs with or without either 50 μM 2-aminoethoxydiphenyl borate (2-APB) to block IP₃ receptors, 20 μM dantrolene to block ryanodine receptors, or both inhibitors. After 18 hours of exposure to AβOs, neurons were stained for MAP2 (green), NeuN (blue), or cyclin D1 (red) to mark neurons that re-entered the cell cycle. A merge of the NeuN, MAP2, and cyclin D1 panels for each treatment are shown at the bottom of FIG. 8A. FIG. 8B is a bar graph of the quantification of the immunofluorescence results. In all drug combinations, there was a significant amount of CCR in samples treated with AβOs, showing that inhibition of ER calcium receptors did not inhibit CCR from occurring. The indicated p values were calculated by one-way ANOVA using the Bonferroni post-hoc test.

### DETAILED DESCRIPTION

The presently disclosed subject matter relates to the discovery that neuronal CCR and excitotoxic calcium influx via NMDAR share a common mechanistic origin initiated by AβOs. Using AβO-treated primary mouse neuron cultures, it is disclosed that CCR was prevented by chelating total cellular calcium, by pharmacologically blocking AβO-mediated calcium influx through NMDAR, or by reducing expression of an N/DAR subunit protein. Moreover, it is disclosed that neuronal CCR in vivo in Tg2576 AD model mice could be blocked by treating the mice prophylactically with memantine.

Taken together, these results mechanistically link AβO-induced calcium influx and neuronal CCR. Moreover, they suggested, unexpectedly, that memantine, which is used as a drug for modestly relieving symptoms in patients with a clinical AD diagnosis but that does not apparently act as a disease-modifying drug in that context, has the potential to forestall disease progression if administered during pre-symptomatic stages of the disease.

Headings are included herein for reference and to aid in locating certain sections. These headings are not intended to limit the scope of the concepts described therein under, and these concepts may have applicability in other sections throughout the entire application.

### I. Abbreviations and Acronyms

2-APB: 2-aminoethoxydiphenyl borate
AβOs: amyloid beta (Aβ) oligomers
AD: Alzheimer's disease
CCR: cell cycle re-entry
HFIP: 1,1,1,3,3,3-hexafluoro-2-propanol
μg/kg body wt.: micrograms per kilogram body weight
Memantine: 1-amino-3,5-dimethyladamantane hydrochloride; also referred to as NAMENDA®
NAMENDA®: also referred to as Memantine
NMDAR: N-methyl-D-aspartate receptor
shRNA: short hairpin RNA

### II. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As use herein, the terms “administration of” and or “administering” a compound should be understood to mean providing a compound of the presently disclosed subject matter or a prodrug of a compound of the presently disclosed subject matter to a subject in need of treatment.

The term “adult” as used herein, is meant to refer to any non-embryonic or non-juvenile subject. For example the term “adult adipose tissue stem cell,” refers to an adipose stem cell, other than that obtained from an embryo or juvenile subject.

Cells or tissue are “affected” by an injury, disease or disorder if the cells or tissue have an altered phenotype relative to the same cells or tissue in a subject not afflicted with the injury, disease, condition, or disorder.

As used herein, an “agonist” is a composition of matter that, when administered to a mammal such as a human, enhances or extends a biological activity of interest. Such effect may be direct or indirect.

A disease, condition, or disorder is “alleviated” if the severity of a symptom of the disease or disorder, the frequency with which such a symptom is experienced by a patient, or both, are reduced.

As used herein, “alleviating an injury, disease or disorder symptom,” means reducing the frequency or severity of the symptom.

As used herein, amino acids are represented by the full name thereof, by the three letter code corresponding thereto, or by the one-letter code corresponding thereto, as indicated in the following table:

| | 3-Letter | 1-Letter | | 3-Letter | 1-Letter |
|---|---|---|---|---|---|
| Full Name | Code | Code | Full Name | Code | Code |
| Aspartic Acid | Asp | D | Threonine | Thr | T |
| Glutamic Acid | Glu | E | Glycine | Gly | G |
| Lysine | Lys | K | Alanine | Ala | A |
| Arginine | Arg | R | Valine | Val | V |
| Histidine | His | H | Leucine | Leu | L |
| Tyrosine | Tyr | Y | Isoleucine | Ile | I |
| Cysteine | Cys | C | Methionine | Met | M |
| Asparagine | Asn | N | Proline | Pro | P |
| Glutamine | Gln | Q | Phenylalanine | Phe | F |
| Serine | Ser | S | Tryptophan | Trp | W |

The term “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

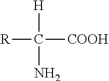

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

An “antagonist” is a composition of matter that when administered to a mammal such as a human, inhibits or impedes a biological activity attributable to the level or presence of an endogenous compound in the mammal. Such effect may be direct or indirect.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

The term “antimicrobial agents” as used herein refers to any naturally-occurring, synthetic, or semi-synthetic compound or composition or mixture thereof, which is safe for human or animal use as practiced in the methods of this presently disclosed subject matter, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a living organism, including skin, hair, tissue, blood, plasma, cells, sweat, and urine.

As used herein, the term “biologically active fragments” or “bioactive fragment” of the polypeptides encompasses natural or synthetic portions of the full-length protein that are capable of specific binding to their natural ligand or of performing the function of the protein.

A “biomarker” is a specific biochemical in the body which has a particular molecular feature that makes it useful for measuring the progress of disease or the effects of treatment, or for measuring a process of interest.

As used herein, the term “carrier molecule” refers to any molecule that is chemically conjugated to the antigen of interest that enables an immune response resulting in antibodies specific to the native antigen.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A”.

Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and in some embodiments at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

The term “complex”, as used herein in reference to proteins, refers to binding or interaction of two or more proteins. Complex formation or interaction can include such things as binding, changes in tertiary structure, and modification of one protein by another, such as phosphorylation.

A “compound,” as used herein, refers to any type of substance or agent that is commonly considered a drug, or a candidate for use as a drug, as well as combinations and mixtures of the above.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, chemokines, protein or peptide hormones, and transforming growth factors. A number of other cytokines are known to those of skill in the art. The sources, characteristics, targets and effector activities of these cytokines have been described.

The term “delivery vehicle” refers to any kind of device or material which can be used to deliver compounds in vivo or can be added to a composition comprising compounds administered to a plant or animal. This includes, but is not limited to, implantable devices, aggregates of cells, matrix materials, gels, nucleic acids, etc.

As used herein, a “derivative” of a compound, when referring to a chemical compound, is one that may be produced from another compound of similar structure in one or more steps, as in replacement of H by an alkyl, acyl, or amino group.

A “derivative protein or peptide,” as used herein, includes any protein or peptide, which in its entirety, or in part, comprises a substantially similar amino acid sequence to an IL-2 and IL-33 fusion peptide (IL233) and has IL233 biological activity as disclosed herein. Derivatives of IL233 may be characterized by single or multiple amino acid substitutions, deletions, additions, or replacements. These derivatives may include (a) derivatives in which one or more amino acid residues are substituted with conservative or non-conservative amino acids; (b) derivatives in which one or more amino acids are added to one or more of the components of the fusion peptide (c) derivatives in which one or more of the amino acids includes a substituent group; (d) derivatives in which one of the constituent groups or a portion thereof is fused to another peptide (e.g., serum albumin or protein transduction domain); (e) derivatives in which one or more nonstandard amino acid residues (i.e., those other than the 20 standard L-amino acids found in naturally occurring proteins) are incorporated or substituted into one of the IL-2 or IL-33 substituents; and (f) derivatives in which one or more nonamino acid linking groups are incorporated into or replace a portion of one of the portions of the fusion protein. A derivative protein may also be referred to as homologous when used in the context described herein. A derivative or homolog as described or claimed herein will have similar activity to IL-2, IL-33, and the fusion peptide IL233 as disclosed herein.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting a disease or disorder or a risk or propensity for development of a disease or disorder, for the types of diseases or disorders encompassed by the presently disclosed subject matter. In any method of diagnosis there exist false positives and false negatives. Any one method of diagnosis does not provide 100% accuracy.

A “disease” is a state of health of an animal wherein the subject cannot maintain homeostasis, and wherein if the disease is not ameliorated then the subject's health continues to deteriorate. In contrast, a “disorder” in an subject is a state of health in which the animal is able to maintain homeostasis, but in which the subject's state of health is less favorable than it would be in the absence of the disorder. Left untreated, a disorder does not necessarily cause a further decrease in the subject's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains. As used herein, the term “effector domain” refers to a domain capable of directly interacting with an effector molecule, chemical, or structure in the cytoplasm which is capable of regulating a biochemical pathway.

The term “downstream” when used in reference to a direction along a nucleotide sequence means the 5′ to 3′ direction. Similarly, the term “upstream” means the 3′ to 5′ direction.

As used herein, an “effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “enhancer” is a DNA regulatory element that can increase the efficiency of transcription, regardless of the distance or orientation of the enhancer relative to the start site of transcription.

As used herein, an “essentially pure” preparation of a particular protein or peptide is a preparation wherein in some embodiments at least about 95%, and in some embodiments at least about 99%, by weight, of the protein or peptide in the preparation is the particular protein or peptide.

As used in the specification and the appended claims, the terms “for example,” “for instance,” “such as,” “including” and the like are meant to introduce examples that further clarify more general subject matter. Unless otherwise specified, these examples are provided only as an aid for understanding the presently disclosed subject matter, and are not meant to be limiting in any fashion.

The terms “formula” and “structure” are used interchangeably herein.

As used herein the term “expression” when used in reference to a gene or protein, without further modification, is intended to encompass transcription of a gene and/or translation of the transcript into a protein.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 2-15 amino acids in length, at least about 15-25 amino acids, at least about 25-50 amino acids in length, at least about 50-75 amino acids in length, at least about 75-100 amino acids in length, and greater than 100 amino acids in length, depending on the particular protein or peptide being referred to.

As used herein, the term “fragment” as applied to a nucleic acid, may in some embodiments be at least about 20 nucleotides in length, in some embodiments at least about 50 nucleotides, in some embodiments from about 50 to about 100 nucleotides, in some embodiments at least about 100 to about 200 nucleotides, in some embodiments at least about 200 nucleotides to about 300 nucleotides, in some embodiments at least about 300 to about 350, in some embodiments at least about 350 nucleotides to about 500 nucleotides, in some embodiments at least about 500 to about 600, in some embodiments at least about 600 nucleotides to about 620 nucleotides, in some embodiments at least about 620 to about 650, and in some embodiments the nucleic acid fragment will be greater than about 650 nucleotides in length.

As used herein, a “functional” molecule is a molecule in a form in which it exhibits a property or activity by which it is characterized. A functional enzyme, for example, is one that exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, the term “induction of apoptosis” means a process by which a cell is affected in such a way that it begins the process of programmed cell death, which is characterized by the fragmentation of the cell into membrane-bound particles that are subsequently eliminated by the process of phagocytosis.

The term “inhibit,” as used herein, refers to the ability of a compound, agent, or method to reduce or impede a described function, level, activity, rate, etc., based on the context in which the term “inhibit” is used. Inhibition can be in some embodiments by at least 10%, in some embodiments by at least 25%, in some embodiments by at least 50%, and in some embodiments, the function is inhibited by at least 75%. The term “inhibit” is used interchangeably with “reduce” and “block”.

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound of the presently disclosed subject matter by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

The term “injected once with a 5-daily dose”, as used herein, means that an induction therapy was initiated wherein mice were injected with 1 μg protein once a day for five consecutive days and then followed over time as indicated.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container which contains the identified compound presently disclosed subject matter or be shipped together with a container which contains the identified compound. Alternatively, the instructional material may be shipped separately from the container with the intention that the instructional material and the compound be used cooperatively by the recipient.

The term “ischemia” as used herein refers to a local anemia due to mechanical obstruction of the blood supply, which gives rise to inadequate circulation of the blood to an organ, tissue, or region of an organ or tissue.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor.

A “receptor” is a compound that specifically binds to a ligand.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

The term “material” refers to any compound, molecule, substance, or group or combination thereof that forms any type of structure or group of structures during or after electroprocessing. Materials include natural materials, synthetic materials, or combinations thereof. Naturally occurring organic materials include any substances naturally found in the body of plants or other organisms, regardless of whether those materials have or can be produced or altered synthetically. Synthetic materials include any materials prepared through any method of artificial synthesis, processing, or manufacture. In some embodiments, the materials are biologically compatible materials.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc., and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “Oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

“Operably linked” refers to a juxtaposition wherein the components are configured so as to perform their usual function. Thus, control sequences or promoters operably linked to a coding sequence are capable of effecting the expression of the coding sequence. By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “per application” as used herein refers to administration of a compositions, drug, or compound to a subject.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer”.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan. As used herein, “pharmaceutical compositions” include formulations for human and veterinary use.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with a pathogenic agent. This is sometimes referred to as induction of tolerance.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer (eds.) The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “prevent,” as used herein, means to stop something from happening, or taking advance measures against something possible or probable from happening. In the context of medicine, “prevention” generally refers to action taken to decrease the chance of getting a disease or condition.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeablyherein.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

A “sample,” as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. RNA interference is a commonly used method to regulate gene expression. This effect is often achieved by using small interfering RNA or short hairpin RNA (shRNA).

As used herein, the term “solid support” relates to a solvent insoluble substrate that is capable of forming linkages (in some embodiments covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, a standard can be a known standard agent or compound which is administered or added to a control sample and used for comparing results when measuring said compound in a test sample. In some embodiments, the standard compound is added or prepared at an amount or concentration that is equivalent to a normal value for that compound in a normal subject. Standard can also refer to an “internal standard,” such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, and/or treatment is an animal. Such animals include mammals. In some embodiments, a subject is a human.

As used herein, a “subject in need thereof” is a patient, animal, mammal, or human, who will benefit from the method of this presently disclosed subject matter.

As used herein, a “substantially homologous amino acid sequence” includes those amino acid sequences which have in some embodiments at least about 95% homology, in some embodiments at least about 96% homology, in some embodiments at least about 97% homology, in some embodiments at least about 98% homology, and in some embodiments at least about 99% homology to an amino acid sequence of a reference sequence. Amino acid sequences similarity or identity can be computed using, for example, the BLASTP and TBLASTN programs which employ the BLAST (basic local alignment search tool) algorithm. The default setting used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially similar nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 96%, 97%, 98%, 99% or more. Substantial similarity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cells united to perform a specific function; (2) a part of an organism consisting of an aggregate of cells having a similar structure and function; or (3) a grouping of cells that are similarly characterized by their structure and function, such as muscle or nerve tissue.

The term “transfection” is used interchangeably with the terms “gene transfer”, “transformation,” and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

The term “transgene” is used interchangeably with “inserted gene,” or “expressed gene” and, where appropriate, “gene”. “Transgene” refers to a polynucleotide that, when introduced into a cell, is capable of being transcribed under appropriate conditions so as to confer a beneficial property to the cell such as, for example, expression of a therapeutically useful protein. It is an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, a “transgenic cell” is any cell that comprises a nucleic acid sequence that has been introduced into the cell in a manner that allows expression of a gene encoded by the introduced nucleic acid sequence.

As used herein, the term “transgenic mammal” means a mammal, the germ cells of which comprise an exogenous nucleic acid.

The term to “treat,” as used herein, means reducing the frequency with which symptoms are experienced by a patient or subject or administering an agent or compound to reduce the frequency with which symptoms are experienced.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease and should be interpreted based on the context of the use.

“Treating” is used interchangeably with “treatment” herein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### III. Compositions and Methods of the Presently Disclosed Subject Matter

As disclosed herein, Aβ oligomer (AβO) biological activities have been shown to be associated with various aspects of the development and progression of Alzheimer's disease (AD). As such, in some embodiments the presently disclosed subject matter provides methods for inhibiting Aβ oligomer (AβO) biological activities, which in some embodiments can relate to treating Alzheimer's disease (AD), inhibiting development and/or progression of at least one symptom associated with AD, inhibiting neuronal cell cycle re-entry (CRR), in some embodiments CCR associated with AD, inhibiting Aβ oligomer (AβO)-stimulated activation of calcium-calmodulin-dependent protein kinase II (CaMKII) biological activity, and/or inhibiting calcium influx-induced excitotoxic neuronal death.

In some embodiments, a subject that is treated with a composition and/or method of the presently disclosed subject matter is pre-symptomatic for AD.

In some embodiments, the subject is APOE4-positive. As set forth herein, the phrase “APOE4-positive” refers to a subject whose genome encodes at least one copy of the F4 allelic variant of the APOE gene. As used herein, the term “APOE4” refers to the F4 allelic variant of the APOE gene. The human APOE genetic locus is on chromosome 19 at 19q13.32. The F4 allelic variant is discussed in U.S. Pat. Nos. 5,508,167; 5,716,828; and 6,027,896; each of which is incorporated by reference in its entirety. APOE4 differs from APOE3 in that in APOE4 arginine is substituted for the normally occurring cysteine at amino acid residue 112. As set forth herein, the F4 allelic variant of the APOE gene is found in ˜25% of humans and ˜50% of confirmed AD patients. Furthermore, all other things being equal, carrying the ε4 allelic variant of the APOE gene increases one's odds of developing AD symptoms ˜5-fold when present as a single gene copy and ˜10-20-fold when two copies are present. APOE4 is by far the strongest genetic risk factor for AD, and since the exemplary inhibitor memantine is an FDA-approved drug with minimal adverse side effects, long term administration of memantine and/or other compositions of the presently disclosed subject matter to APOE4 carriers represents a prophylactic approach to preventing and/or treating AD.

In some embodiments, the presently disclosed methods comprise, consist essentially of, or consist of administering to a subject in need thereof a composition comprising, consisting essentially of, or consisting of an effective amount of an inhibitor of an Aβ oligomer (AβO) biological activity, wherein the inhibitor of the AβO biological activity is an AβO inhibitor, an inhibitor of N-methyl-D-aspartate receptor (NMDAR) signaling, or is a combination thereof.

Various molecules that can modulate biological activities related to NMDAR signaling are known and can be employed within the methods and compositions of the presently disclosed subject matter. Exemplary such molecules include inhibitors of Aβ oligomer (AβO) biological activities and inhibitors of N-methyl-D-aspartate receptor (NMDAR) signaling. Exemplary classes of such molecules include small molecule inhibitors, inhibitory nucleic acids, and calcium chelators, which can be employed individually of in combination.

By way of example and not limitation, an inhibitor of an AβO biological activity and/or of NMDAR signaling can be a small molecule. Exemplary small molecule inhibitors include, but are not limited to 3,5-dimethyladamantan-1-amine (memantine) and (1S,9R)-1-methyl-16-azatetracyclo[7.6.1.0^{2,7}.0^{10,15}]hexadeca-2,4,6,10,12,14-hexaene (MK-801), chlorophenidine, diphenidine, methoxyphenidine, phencyclidine, TCS 46b (CAS 302799-86-6; Santa Cruz Biotechnology, Santa Cruz, Calif.), and ketamine.

As would be understood by those of skill, compounds for use in the compositions and methods of the presently disclosed subject matter can also include compounds that are metabolized by cells (e.g., in vitro or in vivo) to generate an inhibitor of an AβO biological activity and/or of NMDAR signaling (referred to herein as “metabolic precursors”) and compounds that result from metabolic processes by cells (e.g., in vitro or in vivo) on the presently disclosed compounds (referred to herein as “metabolic products”). As would also be understood by one of ordinary skill in the art, the compounds and/or their metabolic precursors or metabolic products that can be employed in the compositions and methods of the presently disclosed subject matter are any that are biologically active, meaning that they function as inhibitors of an AβO biological activity and/or of NMDAR signaling or, upon administration to a cell, tissue, organ, or subject, are metabolized and/or otherwise converted to a molecule that inhibits an AβO biological activity and/or of NMDAR signaling. It is also recognized that the inhibition need not be complete, and that biologically active molecules can be associated with partial inhibition, provided that the result of the partial inhibition provides some therapeutic benefit to the cell, tissue, organ, or subject.

Also included within the scope of the presently disclosed subject matter are derivatives of the presently disclosed inhibitors and pharmaceutically acceptable salts of the presently disclosed inhibitors and/or derivatives. One exemplary derivative of memantine is nitromemantine, which is 3-amino-5,7-diethyladamantan-1-yl nitrate. Other derivatives of memantine and/or other molecules of the presently disclosed subject matter can also be employed, provided that they function as inhibitors of an AβO biological activity and/or of NMDAR signaling or, upon administration to a cell, tissue, organ, or subject, are metabolized and/or otherwise converted to a molecule that inhibits an AβO biological activity and/or of NMDAR signaling.

In addition to small molecule inhibitors, antibodies that bind to proteins involved in NMDAR signaling can also be employed in the compositions and methods of the presently disclosed subject matter. Exemplary such antibodies include those that bind to and inhibit one or more subunits of the NMDAR, including but not limited to the NR1 (also referred to as the GluN1 or GRIN1) component and the GluN2 subunit (of which the NMDAR includes four, referred to as GluN2A-D). Anti-N1 and anti-GuN2 antibodies are commercially available from companies including, but not limited to abcam plc (Cambridge, United Kingdom), Sigma-Aldrich (St. Louis, Mo.), Santa Cruz Biotechnology (Santa Cruz, Calif.), and ARUP Laboratories (Salt Lake City, Utah).

Nucleic acids useful in the presently disclosed subject matter include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; miRNA, siRNA, and the like. The nucleic acids may be prepared by any conventional means typically used to prepare nucleic acids in large quantity. For example, DNAs and RNAs may be chemically synthesized using commercially available reagents and synthesizers by methods that are well-known in the art (see e.g., Gait (1985) Oligonucleotide Synthesis: A Practical Approach (IRL Press, Oxford, England)). RNAs may be produce in high yield via in vitro transcription using plasmids such as SP65 (Promega Corporation, Madison, Wis.).

miRNAs are RNA molecules of about 22 nucleotides or less in length. These molecules are post-transcriptional regulators that bind to complementary sequences on target mRNAs. Although miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules.

Thus, in some embodiments an inhibitor of an AβO biological activity and/or of NMDAR signaling can be a small molecule can be an inhibitory nucleic acid. In some embodiments, the inhibitory nucleic acid targets a nucleic acid encoding a component of an N-methyl-D-aspartate receptor (NMDAR), optionally wherein the component of the NMDAR is an NR1 gene product. The use of inhibitory nucleic acids is exemplified in U.S. Patent Application Publication Nos. 2004/0009949, 2016/0024494, 2016/0362688, and 2020/0063102, each of which is incorporated by reference herein in its entirety.

In some embodiments, the inhibitory nucleic acid is an inhibitory RNA. In some embodiments, the inhibitory RNA targets a human NR1 gene product. As used herein the term “NR1 gene product” refers to a transcription product of an NR1 gene, optionally a human NR1 gene, or a translation product thereof. The human NR1 locus is located on chromosome 9, more particularly 9q34.3, and corresponds to nucleotides 137,139,092-137,168,759 of GENBANK® Accession No. NC_000009.12. Various transcription products of the human NR1 gene are disclosed as GENBANK® Accession Nos. NM_007327.4 (transcript variant GluN1-1a; SEQ ID NO: 1), NM_021569.4 (transcript variant GluN1-2a; SEQ ID NO: 2), NM_001185090.2 (transcript variant GluN1-3b; SEQ ID NO: 3), NM_000832.7 (transcript variant GluN1-4a; SEQ ID NO: 4), and NM_001185091.2 (transcript variant GluN1-5b; SEQ ID NO: 5), any or all of which can be targeted using an inhibitory nucleic acid of the presently disclosed subject matter.

In some embodiments, the inhibitory RNA targets a human NR2A gene product. As used herein the term “NR2A gene product” refers to a transcription product of an NR2A gene, optionally a human NR2A gene, or a translation product thereof. The human NR2A locus is located on chromosome 16, more particularly 16p13.2, and corresponds to the complement of nucleotides 9,753,404-10,182,928 of GENBANK® Accession No. NC_0000016.10. Exemplary transcription products of the human NR2A gene is disclosed as GENBANK® Accession Nos. NM_001134407.3, NM_000833.5, and NM_001134408.2 (SEQ ID NOs: 20-22, respectively).

In some embodiments, the inhibitory RNA targets a human NR2B gene product. As used herein the term “NR2B gene product” refers to a transcription product of an NR2B gene, optionally a human NR2B gene, or a translation product thereof. The human NR2B locus is located on chromosome 12, more particularly 12p13.1, and corresponds to the complement of nucleotides 13,537,337-13,982,012 of GENBANK® Accession No. NC_0000012.12. A transcription product of the human NR2B gene is disclosed as GENBANK® Accession No. NM_000834.5 (SEQ ID NO: 19).

In some embodiments, the inhibitory RNA targets a human NR2C gene product. As used herein the term “NR2C gene product” refers to a transcription product of an NR2C gene, optionally a human NR2C gene, or a translation product thereof. The human NR2C locus is located on chromosome 17, more particularly 17q25.1, and corresponds to the complement of nucleotides 74,842,021-74,860,843 of GENBANK® Accession No. NC_0000016.11. Exemplary transcription products of the human NR2C gene is disclosed as GENBANK® Accession Nos. NM_000835.6, and NM_001278553.1 (SEQ ID NOs: 23 and 24, respectively).

In some embodiments, the inhibitory RNA targets a human NR2D gene product. As used herein the term “NR2D gene product” refers to a transcription product of an NR2D gene, optionally a human NR2D gene, or a translation product thereof. The human NR2D locus is located on chromosome 19, more particularly 19q13.33, and corresponds to nucleotides 48,394,875-48,444,937 of GENBANK® Accession No. NC_0000019.10. An exemplary transcription product of the human NR2D gene is disclosed as GENBANK® Accession No. NM_000836.4 (SEQ ID NO: 25).

In some embodiments, the inhibitory nucleic acid is an shRNA that targets an NR1 gene product. Anti-NR1 shRNAs are commercially available and include those sold by Sigma-Aldrich under the tradename MISSION®. An exemplary anti-NR1 shRNA is Sigma-Aldrich Catalog No. EHU15709, which targets SEQ ID NO: 6, which corresponds to nucleotides 1147-1617 of SEQ ID NOs: 1, 2, and 4, and nucleotides 1210-1680 of SEQ ID NOs: 3 and 5. Other commercially available anti-NR1 inhibitory nucleic acids include those sold under Catalog No. sc-91941 by Santa Cruz Biotechnology.

In some embodiments, the inhibitory nucleic acid is an shRNA that targets an NR2 gene product. Anti-NR2 shRNAs are commercially available and include those sold under Catalog Nos. sc-90622, sc-90622-SH, and sc-90622-V by Santa Cruz Biotechnology.

Alternatively or in addition, the CRISPR/Cas system can be employed to target NR1 and/or NR2. The use of CRISPR/Cas to alter gene expression is described in U.S. Pat. No. 8,697,359 and U.S. Patent Application Publication Nos. 2014/0189896, 2014/0242664, 2014/0287838, and 2014/0357530, each of which is incorporated by reference in its entirety. Commercially available kits to target NR1 by CRISPR/Cas include those sold by GENSCRIPT® (Piscataway, N.J.), which target SEQ ID NOs: 7-12; and by Santa Cruz Biotechnology (Catalog No. sc-405452). Commercially available kits to target NR2 by CRISPR/Cas include those sold by GENSCRIPT® (Piscataway, N.J.; Catalog No. KN423623, which targets NR2B), and by Santa Cruz Biotechnology (Catalog No. sc-411326).

As set forth herein, an inhibitor of an AβO biological activity and/or of NMDAR signaling is an inhibitor of AβO-stimulated activation of a calcium-calmodulin-dependent protein kinase II (CaMKII; also referred to as CAMK2A) biological activity. As used herein, CaMKII refers to a calcium-calmodulin-dependent protein kinase II gene and its products. Exemplary CaMKII genes include the human CaMKII gene, the locus for which is found on human chromosome 5 and corresponds to the complement of nucleotides 150219491-150290130 of GENBANK® Accession No. NC_000005.10. The MaMKII gene products are calcium-dependent serine/threonine kinases that play various roles in synaptic plasticity, learning, and memory, including long-term potentiation via interactions with the NMDAR NR2B subunit (Bayer et al. (2001) Interaction with the NMDA receptor locks CaMKII in an active conformation. Nature 411: 801-805). As such, interfering with CaMKII interactions with the NMDAR can inhibit NMDAR biological activities.

Since CaMKII kinases are calcium-dependent, in some embodiments an inhibitor of an AβO biological activity and/or of NMDAR signaling can be a calcium chelator. Exemplary calcium chelators include 1,2-Bis(2-aminophenoxy)ethane-N,N,N′,N′-tetraacetic acid tetrakis(acetoxymethyl ester) (BAPTA-AM), or a derivative thereof.

Calcium influx into neurons has also been shown to result in excitotoxic neuon death. As set forth herein, in some embodiments excitotoxic neuron death caused by excess calcium influx is initiated by amyloid-β oligomers (AβOs) in Alzheimer's disease (AD). Ectopic neuronal cell cycle re-entry (CCR) is an early symptom of neuron dysfunction, ultimately leading to neuron death, rather than replication of neurons, accounting for substantial neuron death in AD. The present study tests the idea that excitotoxicity and the resulting synaptic dysfunction, in addition to being toxic in their own right, also contribute to the CCR pathway of neuron death. To test this, we first asked whether AβO-mediated calcium influx is necessary for CCR in vitro using primary cortical neurons. Pharmacologically blocking N-methyl-D-aspartate receptor (NMDAR) activity with memantine or MK-801, or knocking down the NMDAR subunit, NR1, blocks CCR.

Furthermore, NMDAR inhibition by these same methods prevents AβO-stimulated activation of calcium-calmodulin-dependent protein kinase II (CaMKII), which mediates excitotoxicity and is necessary for CCR. As demonstrated herein, exposing primary neuron cultures to a 30 second shock of 10 M NMDA induces CCR in a manner similar to AbOs. Additionally, Tg2576 mice treated with memantine blocked CCR compared to the untreated Tg2576 mice and WT controls. As such, a functional connection between NMDAR excitotoxicity and the CCR pathway in AD exists and can be treated and/or prevented using the compositions and methods of the presently disclosed subject matter.

The presently disclosed subject matter is also directed to methods of administering the compounds, cells, proteins and peptides (collectively referred to as compounds) of the presently disclosed subject matter to a subject.

Pharmaceutical compositions comprising the present compounds are administered to an individual in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

The presently disclosed subject matter is also directed to pharmaceutical compositions comprising the peptides of the presently disclosed subject matter. More particularly, such compounds can be formulated as pharmaceutical compositions using standard pharmaceutically acceptable carriers, fillers, solublizing agents and stabilizers known to those skilled in the art.

The presently disclosed subject matter also encompasses the use pharmaceutical compositions of an appropriate compound, homolog, fragment, analog, or derivative thereof to practice the methods of the presently disclosed subject matter, the composition comprising at least one appropriate compound, homolog, fragment, analog, or derivative thereof and a pharmaceutically-acceptable carrier.

The pharmaceutical compositions useful for practicing the presently disclosed subject matter may be administered to deliver a dose of between 1 ng/kg/day and 100 mg/kg/day. Pharmaceutical compositions that are useful in the methods of the presently disclosed subject matter may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the presently disclosed subject matter.

Compounds which are identified using any of the methods described herein may be formulated and administered to a subject for treatment of the diseases disclosed herein.

The presently disclosed subject matter encompasses the preparation and use of pharmaceutical compositions comprising a compound useful for treatment of the conditions, disorders, and diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the presently disclosed subject matter may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the presently disclosed subject matter may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology. A formulation of a pharmaceutical composition of the presently disclosed subject matter suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the presently disclosed subject matter may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Without further description, it is believed that one of ordinary skill in the art can, using the preceding description and the following illustrative EXAMPLES, make and utilize the compounds of the presently disclosed subject matter and practice the claimed methods. The following EXAMPLES therefore, particularly point out exemplary embodiments of the presently disclosed subject matter, and are not to be construed as limiting in any way the remainder of the disclosure.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods for the Examples

Animals and Usage.

All animal usage and protocols were approved by the IACUC of the University of Virginia. Animals were housed in a barrier facility with ad libitum access to food and water on a 12-hour light/dark cycle.

Neuron Dissections and Cultures.

Cortical neuron cultures derived from E17/18 wild type C57/BL6 mouse embryos prepared and maintained as described previously [20]. Cultures were maintained for 16-18 days prior to experimental manipulations.

Aβ Oligomerization and Treatment.

AβOs were made from lyophilized Aβ(1-42) (Anaspec, AS-20276-5), which first was resuspended in 1,1,1,3,3,3-hexafluoro-2-propanol (fIP; Sigma, 105228-5G) and incubated for 4 hours at room temperature to yield a solution of monomeric peptide. 20 μl aliquots were stored at −80° C. until ready to use. To prepare oligomers, an aliquot was dried in a speed vac for 3 hours, solubilized in 1 mM in dimethyl sulfoxide (Sigma, D2650-5X5ML), placed in a water bath sonicator for 5 minutes, and then supplemented with Neurobasal medium (GIBCO/Life Technologies, 21103-049) to yield a final peptide concentration of 100 μM. The solution was then placed on an orbiting rocker at 4° C. for 48 hours, after which it was centrifuged at 14,000×g in a tabletop microcentrifuge for 15 minutes to pellet large aggregates. AβO solutions were diluted into neuron cultures to yield a final total Aβ concentration of ˜2 μM, and the cultures were processed for immunofluorescence microscopy or western blotting 16-18 hours later.

Immunofluorescence Microscopy.

All steps were performed at room temperature unless indicated otherwise. Primary mouse cortical neurons grown on #1.5 thickness, 12 mm round glass coverslips were rinsed once with ice-cold phosphate buffered saline (PBS), and then were fixed in 4% paraformaldehyde in PBS for 12 minutes. Following fixation, the cells were rinsed 3 times for 5 minutes each with ice cold PBS, permeabilized for 15 minutes with PBS containing 0.2% Triton X-100 (Fisher, 9002-93-1), and rinsed 3 times for 5 minutes each with PBS. Next, the cells were blocked with PBST (PBS supplemented with 2% bovine serum albumin (BSA; Roche, 03116956001); and 0.1% Tween 20 (Fisher, 9005-64-5) for 1 hour, after which they were labeled overnight with primary antibodies followed by secondary antibodies for 1 hour. Antibodies were diluted into 2% BSA in PBS, and after each antibody incubation step the cells were rinsed 3 times for 5 minutes each with PBS. Finally, the coverslips were rinsed with ultrapure water and mounted onto slides using Fluormount-G (Southern Biotech, 0100-01) containing 1% 1,4-diazabicyclo[2.2.2]octane (Sigma, D27802-25MG), an anti-quenching agent.

Brain tissue sections were obtained from mice that were transcardially perfused first with 10 ml of phosphate buffer (PB; 0.1 M sodium phosphate, pH 7.6), followed by 10 ml of 4% paraformaldehyde in PB at a flow rate of 1.5 ml/minute. Next, brains were removed and placed in 4% paraformaldehyde in PB for 24 hours, washed 3 times in PBS, incubated in 30% sucrose overnight, and flash frozen using dry ice. Frozen tissue was stored at −80° C. until ready for sectioning to 40 μm thickness on a Microm HM 505 E cryostat.

Sagittal brain sections were placed into wells of a 12-well tissue culture dish, rinsed briefly with PBS, then rinsed 3 times for 15 minutes each in PBS+0.3% Triton X-100 followed by PBS+0.3% Triton X-100 and 2% normal goat serum for 2 hours with gentle rocking. Sections were then labeled with primary and secondary antibodies as described for cultured neurons.

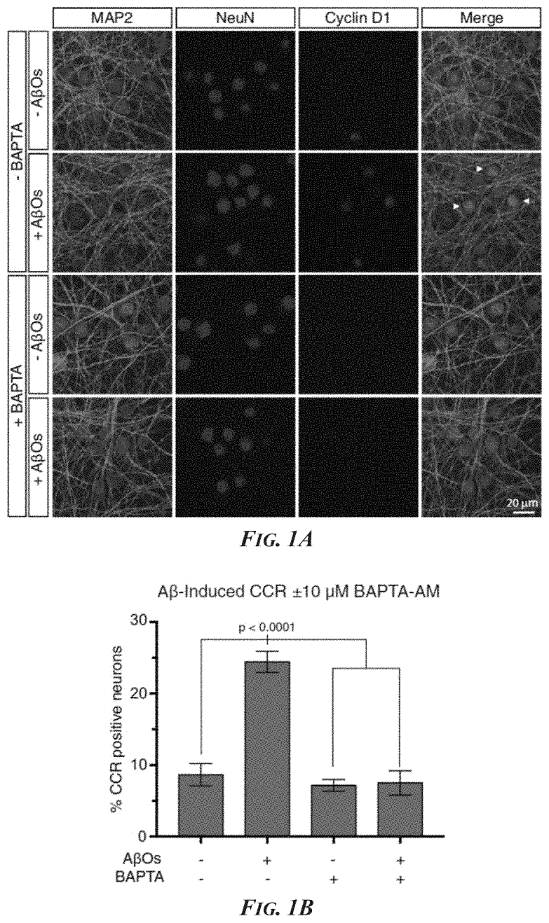

Cultured neurons and brain sections were imaged on either of two microscopes: 1) Nikon Eclipse Ti equipped with a Yokogawa CSU-X1 spinning disk head, 20×0.75 NA CFI Plan Apo, 40×1.3 NA CFI S Fluor and 60×1.4 NA Plan Apo objectives; 405 nm, 488 nm, 561 nm and 640 nm lasers; and a Hamamatsu Flash 4.0 scientific CMOS camera; or 2) EVOS FL imaging system (Invitrogen). Micrographs were captured using either Nikon Elements or EVOS software. CCR-positive cells were counted using the ImageJ cell counter plugin for brain sections (available at an NIH website), and were counted manually using the EVOS microscope for primary neuron cultures. For cultured neurons (FIGS. 1-4, S5 and S2), 4 biological replicates of >300 neurons were counted per condition per experiment.

For brain sections (FIG. 5), 4 brains from each condition (WT with and without memantine, and Tg2576 with and without memantine) were used. To ensure thorough neuroanatomical coverage, 3 images for each of 3 cortical sections (lateral, medial, middle) were counted per brain to quantify cyclin D1-positive neurons. ˜500 neurons per section were counted, yielding ˜1,500 each of lateral, medial and middle cortical neurons, or a total of ˜4,500 neurons per mouse. Because 4 brains per condition were counted in this manner, ˜18,000 total neurons were counted for each condition. At a qualitative level, CCR neurons appeared to be evenly distributed throughout the cortical regions described here.

Western Blotting.

Samples were resolved on 12% SDS polyacrylamide gels, then transferred at 100 V for 1.75 hours onto 0.22 μm pore size nitrocellulose membranes using a Bio-Rad Mini-PROTEAN Tetra Electrophoresis Cell. Following transfer, blots were rinsed once in tris-buffered saline (TBS), blocked for 30-60 minutes in blocking buffer (LI-COR, 927-50000), sequentially incubated in primary antibodies overnight at 4° C. in antibody buffer (1:1 mix of blocking buffer, and TBST: TBS+0.1% Tween-20), then LI-COR infrared-labeled secondary antibodies for 30 minutes. Blots were rinsed 3 times for 5 minutes each using TBST after the primary antibody or TBS after the secondary antibody. Finally, membranes were scanned using a LI-COR Odyssey imaging station.

Lentivirus Production.

HEK-293T cells were grown in culture to 90% confluency, then transfected with envelope, packaging, and shRNA plasmids using Lipofectamine 3000 (Invitrogen, L3000-015). MISSION® brand shRNAs for the knockdown vectors were obtained from Sigma-Aldrich (NR1 knockdown vector: TRCN0000233327). Media collected at 2-3 and 4-5 days post-transfection was pooled, and virus was pelleted in a Beckman Coulter Optima LE-80K ultracentrifuge for 2 hours at 23,000 rpm (95,389×gₘₐₓ) at 4° C. in an SW28 rotor. The supernatant was then removed and the pellets containing the virus were resuspended in 300 μl Neurobasal medium. Viruses were then aliquoted and stored at −80° C. until ready for use.

Memantine Treatment.

Tg2576 AD model mice that overexpress human β-amyloid precursor protein (APP) with the K670N/M67IL Swedish mutation [27] and WT mice of the same background (50% SLW, 50% C57/BL6) were treated with memantine at ˜30 mg/kg/day based on a previously described protocol [28] from the time they were weaned (3 weeks) until 2 months of age. The memantine concentration in water, ˜1 mM, chosen based on mouse weight and the assumption of mice consuming 0.15 ml of water per g body weight per day. Mice were given ad libitum access to food and water.

Statistics.

All statistical analysis was performed with Prism 7 software. For BAPTA-AM, MK-801, memantine and knockdown experiments, one-way ANOVAs with Bonferroni post-hoc tests were used for analysis. For the phosphorylated calcium-calmodulin-dependent protein kinase II (pCaMKII) time course, a two-way ANOVA with Bonferroni post-hoc test was used to give p values for individual time points. For Tg2576 memantine experiments, one-way ANOVA with Bonferroni post-hoc test was used. At least 4,000 neurons were counted per mouse per condition.

Reagents Used.

Drugs: 2-Aminoethoxydiphenyl borate (2-ABP: Sigma; D9754-10G), BAPTA-AM (ThermoFisher; B1205), 6-cyano-7-nitroquinoxaline-2,3-dione (CNQX: Sigma; C239-100MG), dantrolene (Sigma; D9175-100MG), MK-801 (Sigma; M107-5MG), memantine (Sigma; M9292-100MG). Antibodies: chicken anti-MAP2 (Abcam; ab92434), mouse anti-NeuN (Millipore; MAB377), rabbit anti-CyclinDI (Abcam; ab16663), rabbit anti-NR1 (CST; D65B7), rabbit anti-pCaMKII (CST; D21E4), mouse anti-total CaMKII (BD Labs; 611292), mouse anti-βIII-tubulin; (TuJ; courtesy of T. Spano and T. Frankfurter, University of Virginia), goat anti-mouse IgG Alexa Fluor 568 (Life Technologies; A11041), goat anti-mouse IgG Alexa Fluor 405 (Invitrogen; 35501BID), goat anti-rabbit IgG Alexa Fluor 488 (Life Technologies; A11034), goat anti-chicken IgG Alexa Fluor 647 (Life Technologies; A21235), goat anti-mouse IRDye 680LT (Licor; 925-68070), goat anti-rabbit IRDye 800 CW (Licor; 925-32211.

### Example 1

### Intracellular Calcium is Necessary for CCR

To test the hypothesis that neuronal CCR is calcium-dependent we first treated primary mouse cortical neurons with BAPTA-AM, a cell-permeant chelator of intracellular calcium, beginning 30 minutes prior to addition of AβOs to the cultures. After 16-18 hours of AβO exposure, neurons were fixed and stained by triple immunofluorescence microscopy with antibodies to the G1 marker, cyclin D1, the neuron-specific nuclear protein, NeuN, and the neuronal somatodendritic protein, MAP2. As shown in FIG. 1, AβO treatment increased the fraction of cyclin D1-positive neurons from ˜8% to ˜25%, but the rise in cyclin D1-positive neurons was prevented by pre-treatment with BAPTA-AM. These results demonstrate that calcium is necessary for neuronal CCR.

### Example 2

### CCR is Blocked by Pharmacologically Inhibiting NMDAR, but not AMPA Receptor or ER Calcium Stores

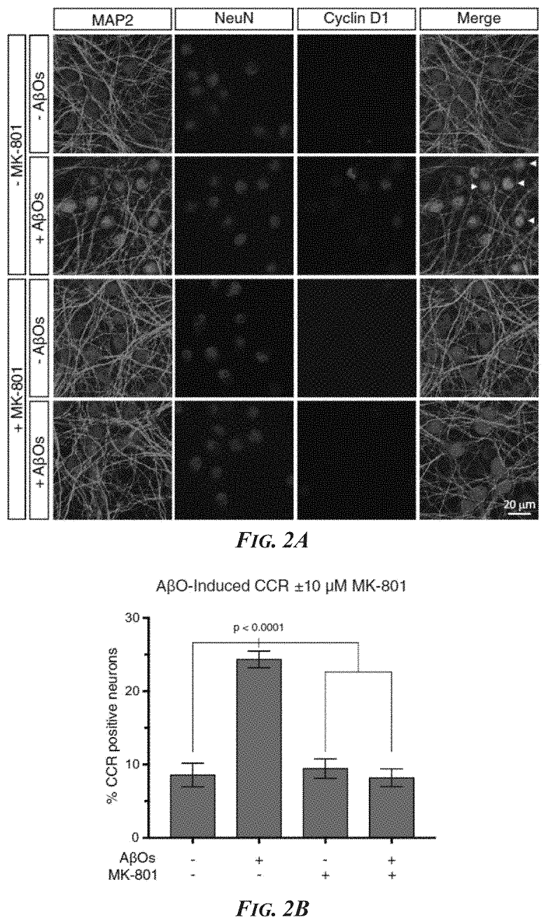

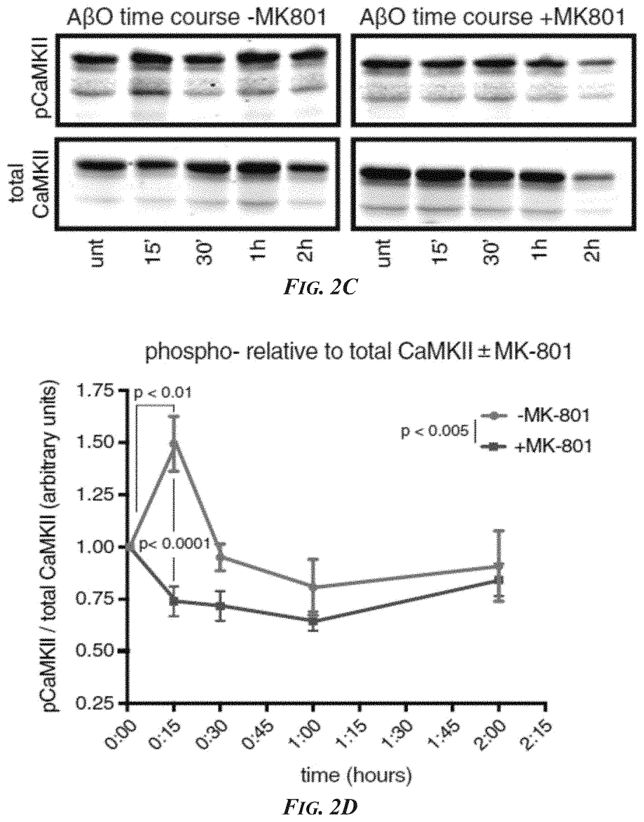

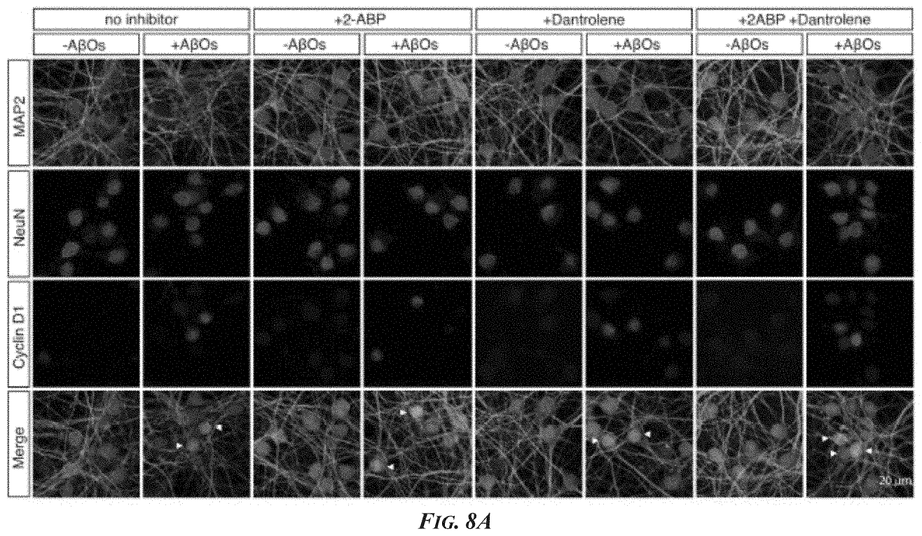

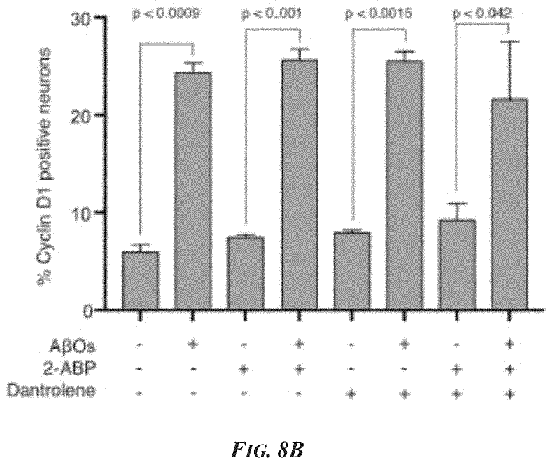

Which specific calcium sources contribute to the initiation of CCR were determined. Given the numerous deficits induced by AβOs at the synapse, and that AβOs induce calcium influx through NMDAR, whether this AβO-mediated calcium influx is required for CCR was investigated. Accordingly, primary neuron cultures were treated with MK-801, an NMDAR inhibitor that blocks calcium entry through the channel pore, beginning 30 minutes before AβOs were added. After 16-18 hours of AβO treatment, neurons were fixed and stained for NeuN, MAP2 and cyclin D1 to enable quantitation of neuronal CCR. As shown in FIG. 2A/B, pre-treatment of neurons with MK-801 blocked the induction of CCR by AβOs. Otherwise identical experiments were performed using 6-cyano-7-nitroquinoxaline-2,3-dione (CNQX) to block plasma membrane AMPA receptors (FIG. 7), 2-aminoethoxydiphenyl borate (2-ABP) to block ER-associated IP₃ receptors and TRP channels (FIG. 8), or dantrolene to inhibit ER-associated ryanodine receptors (FIG. 8). Because none of those inhibitors blocked CCR, it appeared that NMDAR was the major, and possibly exclusive source of the excess calcium that enters neurons in response to AβO exposure and initiates CCR.

Previous work from our lab demonstrated that AβO-mediated CCR requires activation of at least four protein kinases: CaMKII, the src-family kinase, fyn, protein kinase A (PKA) and mTOR [19, 20]. CaMKII is of particular interest, as it can be activated via calcium influx through NMDAR. We thus tested the hypothesis that AβO-mediated calcium influx through NMDAR activates CaMKII as a mediator of CCR. Again, we treated primary neurons with AβOs with or without a 30 minute MK-801 pre-treatment, and collected the cells at various time points from 0-2 hours after AβO stimulation for western blotting with antibodies to phospho-activated and total CaMKII. As shown in FIG. 2C/D, we found that CaMKII was transiently activated 15 minutes after AβO addition, and that activation was prevented by MK-801. The AβO-mediated activation of CaMKII necessary for CCR [19] is therefore dependent upon calcium influx through NMDAR.

### Example 3

### Knockdown of NR1 Blocks CCR

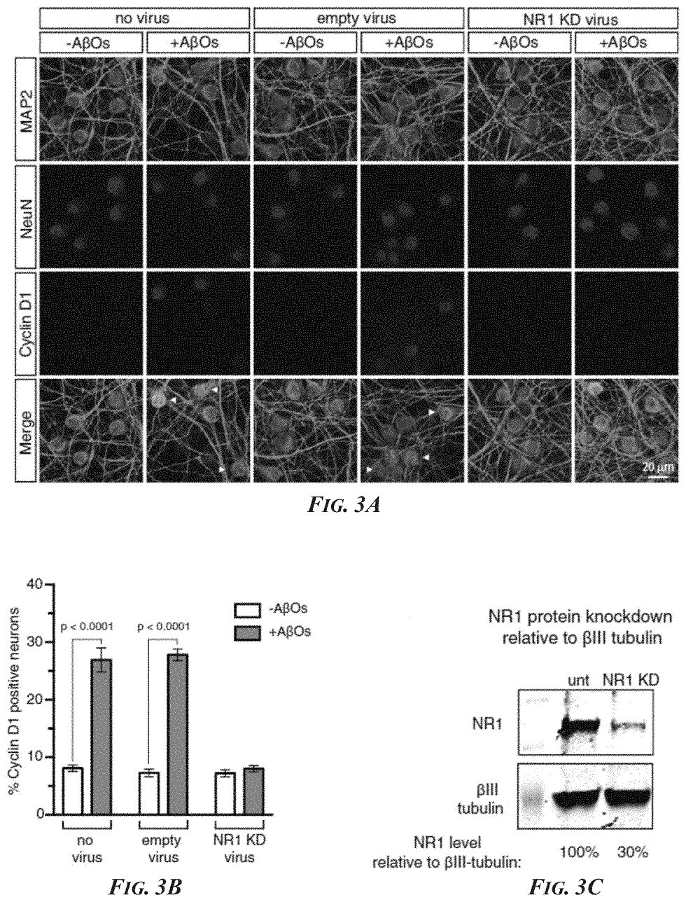

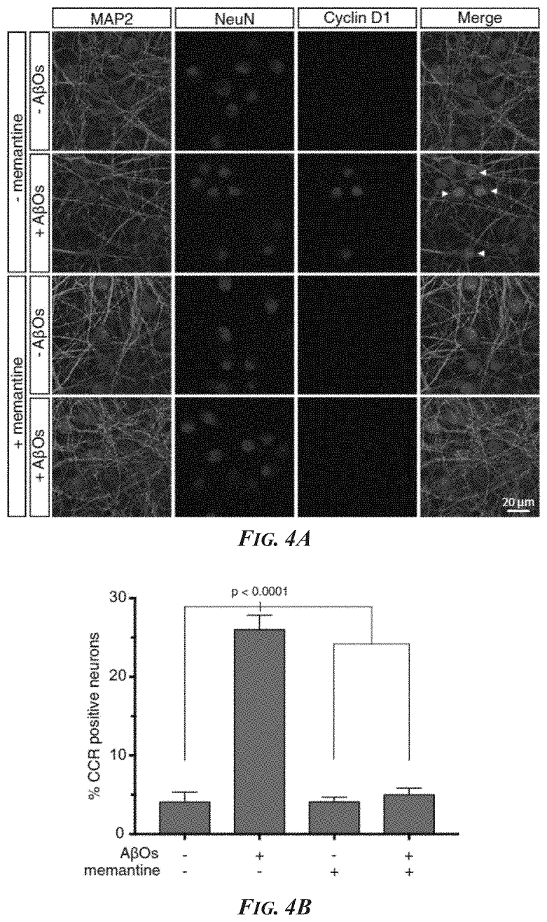

To provide independent, non-pharmacological evidence for the role of NMDAR in AβO-stimulated neuronal CCR, we used antisense shRNA to knock down expression of the constitutive NMDAR subunit, NR1. 96 hours prior to AβO addition, primary neuron cultures were transduced with lentivirus containing an empty vector, or an shRNA vector to NR1. Following a 16-18 hour exposure to AβOs, the cells were processed for triple immunofluorescence with antibodies to MAP2, NeuN, and cyclin D1. As shown in FIG. 3, we found that reducing the neuronal level of NR1 to 30% of normal blocked the ability of AβOs to cause CCR. These results validate the pharmacological evidence based on the use of MK-801 (FIG. 2) that NMDAR is essential for enabling AβOs to drive normally post-mitotic neurons back into the cell cycle.

### Example 4

### Memantine Blocks CCR in Cultured Neurons and In Vivo

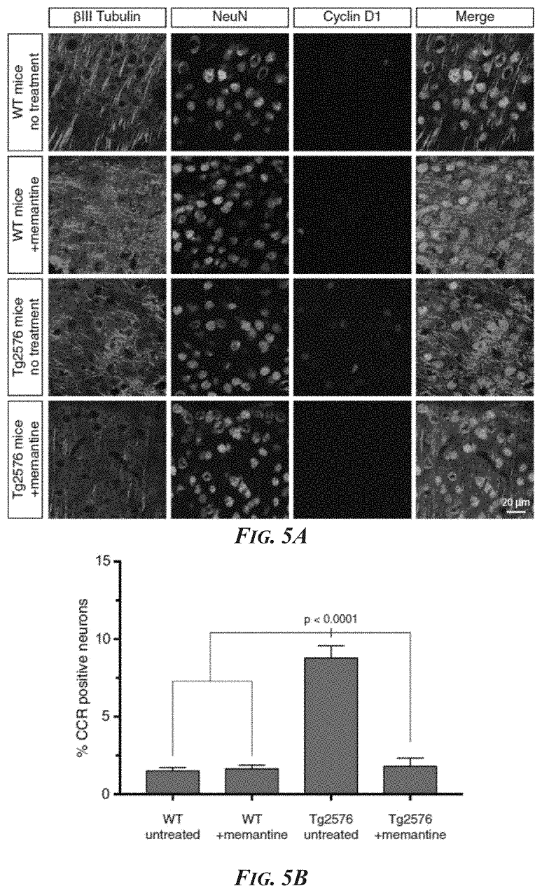

Memantine is an FDA-approved drug for AD treatment, and works by preventing excess calcium influx through NMDAR while still allowing normal calcium-mediated synaptic transmission through the receptor. To gain further insight into the mechanism by which excess calcium induces CCR, we tested whether specifically blocking excess, but not normal calcium entry through NMDAR could block CCR. Similar to experiments with MK-801, we treated primary neurons with AβOs for 16-18 hours with or without a 30 minute pre-treatment with memantine, followed by triple immunofluorescence labeling with antibodies to NeuN, MAP2 and cyclin D1. As shown in FIG. 4, memantine, like MK-801 (FIG. 2), prevented AβOs from inducing neuronal CCR. Although memantine is typically used to treat late stage AD and is not considered to be a disease-modifying drug, these results with cultured neurons raised the possibility that memantine can interfere with CCR in vivo and might therefore be able to block neuron death in AD.

To test that possibility, wild type (WT) and Tg2576 AD model mice [27], which overexpress human APP with the Swedish mutation (K670M/N671L), were provided ad libitum access to memantine-containing water from the time they were weaned (3 weeks) until 2 months of age, when abundant neuronal CCR is normally evident in the Tg2576 strain [20]. Following the 5 weeks of memantine treatment, the animals were euthanized and brain sections were stained with antibodies to NeuN, cyclin D1 and the neuron-specific protein, 111-tubulin. As shown in FIG. 5, WT mice with or without memantine treatment had a basal level of 1.6% neuronal CCR along cortical regions. as determined by cyclin D1 immunoreactivity. In contrast, the basal level of CCR in similar cortical regions of Tg2576 mice was 8.8%, which was reduced to WT levels by memantine. These results show that treating Tg2576 mice with memantine before symptom onset acts prophylactically to prevent CCR.

### Discussion of the Examples

The behavioral symptoms of AD are directly caused by the impaired function and loss of synapses by neurons that control memory and cognition, and by the death of those neurons. Using CCR as a surrogate for eventual neuronal death in primary neuron cultures and in vivo, we demonstrate here that AβO-stimulated entry of excess calcium via NMDAR, which has been shown by others to trigger synaptotoxicity in vivo [3,29,30], also initiates neuronal CCR. We found that AβO-induced CCR could be prevented in cultured neurons by chelating total intracellular calcium with BAPTA-AM (FIG. 1), blocking calcium entry via NMDAR with MK-801 (FIG. 2), shRNA knockdown of the constitutive NMDAR subunit NR1 (FIG. 3), or using memantine for specifically blocking the entry of excess calcium into neurons via NMDAR (FIG. 4). In contrast, inhibitors of cytoplasmic calcium elevation by AMPA receptor (FIG. 7) or ER calcium stores (FIG. 8) did not block AβO-induced CCR. Most importantly in terms of clinical relevance, we also show that memantine treatment of Tg25756 AD model mice blocks neuronal CCR in vivo (FIG. 5). Together, these results indicate that AβO-stimulated excitotoxic calcium influx through NMDAR and CCR/cell death share a common mechanistic origin. Furthermore, they suggest that memantine, which is widely considered to relieve symptoms without attacking processes that lead to neuronal decline, may indeed slow or prevent AD progression if administered at early, pre-symptomatic disease stages.

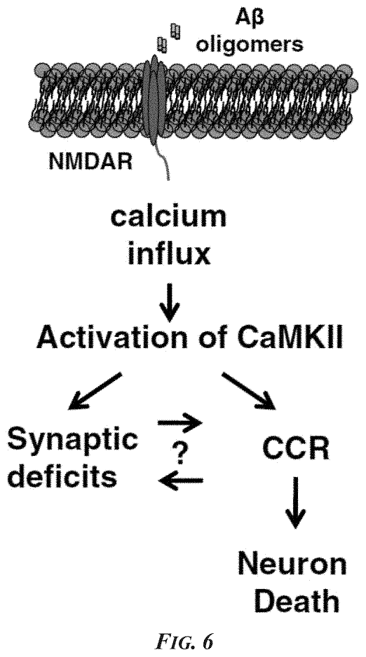

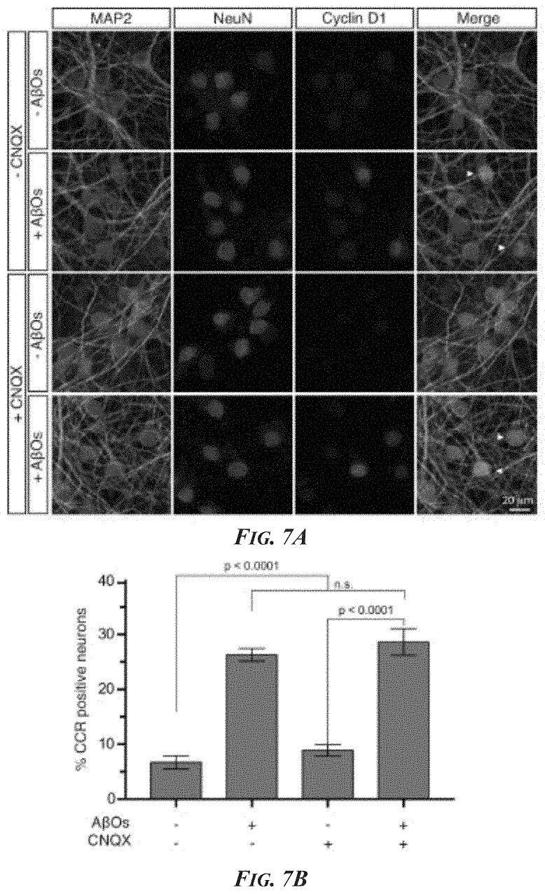

While the pathways for both synaptotoxicity [11, 32] and CCR/cell death proceed following AβO-stimulated entry of excess calcium via NMDAR, it is not yet clear if these dual causes of AD symptoms are connected serially or represent bifurcating processes with a common origin. As illustrated in FIG. 6, AβO-mediated synaptic deficits stemming from aberrant NMDAR calcium influx could lead to CCR in a linear pathway, or alternatively, AβO-mediated calcium influx could contribute to synaptic deficits and CCR independently, eventually causing neuron death. These two schemes are not necessarily mutually exclusive, and further work will be needed to resolve this issue.

It has been known since the 1990s that vulnerable neurons in AD frequently enter into a paradoxical pathway before dying: ectopic CCR. Differentiated neurons are permanently post-mitotic, but in AD and several other neurodegenerative disorders, neuron death often appears to follow expression of various cell markers, such as cyclin D1 and duplication of large portions of the genome [18,31]. Furthermore, studies of human brain have shown a peak in polyploidy between preclinical AD and mild AD, followed by a decline in polyploidy and neuronal numbers in severe AD, presumably because CCR neurons preferentially die [17].

AβOs are the dominant species causing the excess calcium influx that contributes to excitotoxicity in AD, particularly through NMDARs [32,33]. The data shown here imply that excitotoxicity and CCR are mechanistically connected to NMDAR activity by tau. For instance, tau plays an essential role in effects downstream of excitotoxic calcium influx, as knocking out tau in an AD model mouse model protects against learning and memory deficits, seizures and premature death [34]. Additionally, it has been shown that NMDAR can be influenced by tau upstream of calcium influx, by recruiting the non-receptor tyrosine kinase, fyn, which phosphorylates NMDAR and thereby facilitates excess calcium influx provoked by AβOs [3,29,35]. We have also shown tau to be necessary for CCR to occur by a mechanism that requires fyn-dependent tau phosphorylation [19]. Although our results imply that excess calcium entry into neurons via NMDAR is sufficient to drive CCR, they do not exclude possible contributions to cytoplasmic calcium level increases from at least one other source, mitochondria [36].

There are additional ways that calcium is likely to be connected to CCR. Calcium is one of the most versatile second messengers [37], not only for controlling functions of post-mitotic neurons, but also in regulating division of proliferative cells. In fact, calcium is involved at multiple stages of the cell cycle, including conversion from the quiescent state of G0 to G1, and from G1 to S phase [38,39]. These transitions are germane to neuronal CCR, because neurons undergoing CCR apparently proceed into S phase before their eventual death. In other excitotoxic paradigms, such as stroke and ischemia, NMDAR overexcitation by kainic acid treatment is sufficient to induce CCR in neurons before their eventual death by excitotoxic shock [40-42]. It is possible that this already established pathway has been repurposed in the neuron as a response to stress or toxicity.

Memantine alleviates many AD symptoms, including loss of LTP/LTD, learning and memory deficits [28,43], synapse loss, and abnormal tau phosphorylation [44]. Memantine is additionally protective against more canonic excitotoxic insults beyond AD, including preventing neuron damage after ischemic shock in mice and rats [45,46]. Our results show that memantine also works prophylactically to prevent CCR when used before disease signs are evident. This raises the possibility that memantine might slow or halt disease progression if administered at a sufficiently early pre-symptomatic disease stage. With that idea in mind and because memantine is a safe, FDA-approved drug, its long term administration to non-symptomatic, ApoE4-positive individuals as a prophylactic against AD is an aspect of the presently disclosed subject matter.

For example, memantine and/or its precursors, metabolic products, and derivatives can be provided to individuals who are judged to be at risk of developing AD symptoms based on assays for disease-related biomarkers.

The symptoms of Alzheimer's disease (AD) are caused by the deterioration and loss of synapses among neurons that mediate memory and cognition, and by the death of those neurons. Currently, there are five FDA-approved drugs for AD, four of which are cholinesterase inhibitors, and the fifth of which, memantine (NAMENDA®), is an NMDA receptor antagonist. Each of these drugs is administered to relieve symptoms in patients with confirmed AD diagnoses, but none of them have been considered to interfere with synapse failure or neuron death, and they are therefore not considered to be disease modifying. Indeed, the NAMENDA® product sheet provided by its manufacturer, Allergan, states: “There is no evidence that memantine prevents or slows neurodegeneration in patients with Alzheimer's disease”.

As such, there are no credible expectations that the cholinesterase inhibitors can be disease modifying under any circumstances, but as disclosed herein, evidence has been obtained that memantine might be different. Ectopic neuronal cell cycle re-entry (CCR), is widespread in AD but does not result in the creation of new neurons by cell division. Ironically, CCR instead leads to neuron death, which occurs on a massive level in AD. Approximately 30% of frontal lobe neurons will have died by the time a typical patient finally passes away, and up to 90% of the neuron death in AD is thought to be caused by CCR.

Using cultured neurons and transgenic AD model mice, it was first discovered that neuronal CCR is initiated by amyloid-β oligomers (AβOs), the precursors of the amyloid plaques that characteristically accumulate in AD brain, and proceeds by a mechanism dependent on tau, the building blocks of the neurofibrillary tangles that are also a histopathological hallmark of AD brain (Seward et al. (2013) J Cell Science 126:1278-1286).

Recently, it was determined that memantine blocks CCR, not only in AβO-treated cultured neurons, but in an AD model mouse strain as well. These new data raise the distinct possibility that memantine actually has disease modifying properties and might thus be able to retard AD progression—provided it is administered before overt signs of disease are evident. See Kodis et al. (2018) N-methyl-D-aspartate receptor-mediated calcium influx connects amyloid-β oligomers to ectopic neuronal cell cycle reentry in Alzheimer's disease. Alzheimer's & Dementia 14(10):1302-1312, which is incorporated herein by reference in its entirety.

Summarily, Alzheimer's disease (AD) symptoms reflect synaptic dysfunction and neuron death. Amyloid-β oligomers (AβOs) induce excess calcium entry into neurons via N-methyl-D-aspartate receptors (NMDARs), contributing to synaptic dysfunction. The presently disclosed subject matter relates in some embodiments to the discovery that AβO-stimulated calcium entry also drives neuronal cell cycle re-entry (CCR), a prelude to neuron death in AD. Pharmacologic modulators of calcium entry and gene expression knockdown were used in cultured neurons and AD model mice. In cultured neurons, AβO-stimulated CCR was blocked by NMDAR antagonists, total calcium chelation with BAPTA-AM, or knockdown of the NMDAR subunit, NR1. NMDAR antagonists also blocked activation of calcium-calmodulin-dependent protein kinase II (CaMKII), and treatment of Tg2576 AD model mice with the NMDAR antagonist, memantine, prevented CCR. Thus, the data presented herein demonstrated a role for AβO-stimulated calcium influx via NMDAR and CCR in AD, and provides a basis for administering memantine and/or other compositions of the presently disclosed subject matter as disease-modifying therapies for pre-symptomatic AD.

### REFERENCES

All references listed below, as well as all references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.

Other than their use in chemical formulae, numbers in brackets throughout the application correspond to the references listed herein below:
[1] Sheng et al. (2012) Synapses and Alzheimer's Disease. Cold Spring Harbor Perspectives in Biology 4:a005777-7.
[2] Selkoe (2002) Alzheimer's Disease Is a Synaptic Failure. Science 298:789-91.
[3] Ittner et al. (2010) Dendritic function of tau mediates amyloid-beta toxicity in Alzheimer's disease mouse models. Cell 142:387-97.
[4] de la Monte (2014) Type 3 diabetes is sporadic Alzheimer's disease: mini-review. European Neuropsychopharmacology 24:1954-60.
[5] Vossel et al. (2010) Tau reduction prevents Abeta-induced defects in axonal transport. Science 330:198.
[6] Rapoport et al. (2002) Tau is essential to β-amyloid-induced neurotoxicity. Proc Natl Acad Sci USA 99:6364-9.
[7] Bloom (2014) Amyloid-O and tau: the trigger and bullet in Alzheimer disease pathogenesis. JAMA Neurol 71:505-8.
[8] Nussbaum et al. (2012) Prion-like behaviour and tau-dependent cytotoxicity of pyroglutamylated amyloid-0. Nature 485:651-5.
[9] Hardy & Selkoe (2002) The amyloid hypothesis of Alzheimer's disease: progress and problems on the road to therapeutics. Science 297:353-6.
[10] Walsh et al. (2002) Naturally secreted oligomers of amyloid beta protein potently inhibit hippocampal long-term potentiation in vivo. Nature 416:535-9.
[11] Lacor et al. (2007) A Oligomer-Induced Aberrations in Synapse Composition, Shape, and Density Provide a Molecular Basis for Loss of Connectivity in Alzheimer's Disease. J Neurosci 27:796-807.
[12] Herrup & Yang (2007) Cell cycle regulation in the postmitotic neuron: oxymoron or new biology? Nature Rev Neurosci 8:368-78.
[13] Arendt et al. (2010) Selective Cell Death of Hyperploid Neurons in Alzheimer's Disease. The American Journal of Pathology 177:15-20.
[14] Yang et al. (2003) Neuronal cell death is preceded by cell cycle events at all stages of Alzheimer's disease. J Neurosci 23:2557-63.
[15] Greene et al. (2007) Cell cycle molecules define a pathway required for neuron death in development and disease. Biochim Biophys Acta 1772:392-401.
[16] Varvel et al. (2008) Abeta oligomers induce neuronal cell cycle events in Alzheimer's disease. J Neurosci 28:10786-93.
[17] Arendt (2012) Cell cycle activation and aneuploid neurons in Alzheimer's disease. Mol Neurobiol 46:125-35.
[18] Yang et al. (2001) DNA replication precedes neuronal cell death in Alzheimer's disease. J Neurosci 21:2661-8.
[19] Seward et al. (2013) Amyloid-O signals through tau to drive ectopic neuronal cell cycle re-entry in Alzheimer's disease. J Cell Sci 126:1278-86.
[20] Norambuena et al. (2017) mTOR and neuronal cell cycle reentry: How impaired brain insulin signaling promotes Alzheimer's disease. Alzheimer's & Dementia 13:152-67.
[21] Khachaturian (1989) Calcium, membranes, aging, and Alzheimer's disease. Introduction and overview. Annals of the NY Academy of Science 568:1-4.
[22] Mattson et al. (1992) β-Amyloid peptides destabilize calcium homeostasis and render human cortical neurons vulnerable to excitotoxicity. J Neurosci 12:376-89.
[23] Workgroup1 AACH (2017) Calcium Hypothesis of Alzheimer's disease and brain aging: A framework for integrating new evidence into a comprehensive theory of pathogenesis. Alzheimer's & Dementia 13:178-182.e17.
[24] Wang & Qin (2010) Molecular and cellular mechanisms of excitotoxic neuronal death. Apoptosis 15:1382-402.
[25] Malinow (2011) New developments on the role of NMDA receptors in Alzheimer's disease. Current Opinion in Neurobiology 22:559-63.
[26] Lipton (2006) Paradigm shift in neuroprotection by NMDA receptor blockade: Memantine and beyond. Nature Reviews Drug Discovery 5:160-70.
[27] Hsiao et al. (1996) Correlative memory deficits, Abeta elevation, and amyloid plaques in transgenic mice. Science 274:99-102.
[28] Minkeviciene (2004) Memantine Improves Spatial Learning in a Transgenic Mouse Model of Alzheimer's Disease. Journal of Pharmacology and Experimental Therapeutics 311:677-82.
[29] Roberson et al. (2011) Amyloid-β/Fyn-Induced Synaptic, Network, and Cognitive Impairments Depend on Tau Levels in Multiple Mouse Models of Alzheimer's Disease. J Neurosci 31:700-11.
[30] Mucke & Selkoe (2012) Neurotoxicity of Amyloid β-Protein: Synaptic and Network Dysfunction. Cold Spring Harbor Perspectives in Medicine 2:a006338-8.
[31] Vincent et al. (1997) Aberrant expression of mitotic cdc2/cyclin B1 kinase in degenerating neurons of Alzheimer's disease brain. J Neurosci 17:3588-98.
[32] De Felice et al. (2007) Abeta Oligomers Induce Neuronal Oxidative Stress through an N-Methyl-D-aspartate Receptor-dependent Mechanism That Is Blocked by the Alzheimer Drug Memantine. Journal of Biological Chemistry 282:11590-601.
[33] Alberdi et al. (2010) Amyloid Roligomers induce Ca2+ dysregulation and neuronal death through activation of ionotropic glutamate receptors. Cell Calcium 47:264-72.
[34] Roberson et al. (2007) Reducing Endogenous Tau Ameliorates Amyloid β-Induced Deficits in an Alzheimer's Disease Mouse Model. Science 316:750-4.
[35] Zempel et al. (2010) A Oligomers Cause Localized Ca2+ Elevation, Missorting of Endogenous Tau into Dendrites, Tau Phosphorylation, and Destruction of Microtubules and Spines. J Neurosci 30:11938-50.
[36] Bezprozvanny & Mattson (2008) Neuronal calcium mishandling and the pathogenesis of Alzheimer's disease. Trends in Neurosciences 31:454-63.
[37] Berridge et al. (2000) The versatility and universality of calcium signalling. Nature Reviews Molecular Cell Biology 1:11-21.
[38] Chafouleas et al. (1984) Changes in calmodulin and its mRNA accompany reentry of quiescent (G0) cells into the cell cycle. Cell 36:73-81.
[39] Machaca (2010) Ca2+ signaling, genes and the cell cycle. Cell Calcium 48:243-50.
[40] Kuan (2004) Hypoxia-Ischemia Induces DNA Synthesis without Cell Proliferation in Dying Neurons in Adult Rodent Brain. J Neurosci 24:10763-72.
[41] Wen et al. (2005) Cell-cycle regulators are involved in transient cerebral ischemia induced neuronal apoptosis in female rats. FEBS Letters 579:4591-9.
[42] Marathe et al. (2015) Notch signaling in response to excitotoxicity induces neurodegeneration via erroneous cell cycle reentry. Cell Death & Differentiation 22:1775-84.
[43] Martinez-Coria et al. (2010) Memantine improves cognition and reduces Alzheimer's-like neuropathology in transgenic mice. The American Journal of Pathology 176:870-80.
[44] Li et al. (2004) Memantine inhibits and reverses the Alzheimer type abnormal hyperphosphorylation of tau and associated neurodegeneration. FEBS Letters 566:261-9.
[45] Seif el Nasr et al. (1990) Neuroprotective effect of memantine demonstrated in vivo and in vitro. Eur J Pharmacol 185:19-24.
[46] López-Valdés et al. (2014) Memantine enhances recovery from stroke. Stroke 45:2093-100.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. (canceled)

2. A method for inhibiting development and/or progression of at least one symptom associated with Alzheimer's disease (AD), the method comprising, consisting essentially of, or consisting of administering to a subject in need thereof a composition comprising, consisting essentially of, or consisting of an effective amount of an inhibitor of an Aβ oligomer (AβO) biological activity.

3. (canceled)

4. A method for inhibiting Aβ oligomer (AβO) biological activity, the method comprising, consisting essentially of, or consisting of administering to a subject in need thereof a composition comprising, consisting essentially of, or consisting of an effective amount of an inhibitor of N-methyl-D-aspartate receptor (NMDAR) signaling.

5. (canceled)

6. A method for inhibiting calcium influx-induced excitotoxic neuronal death, the method comprising, consisting essentially of, or consisting of administering to a subject in need thereof a composition comprising, consisting essentially of, or consisting of an effective amount of an inhibitor of an Aβ oligomer (AβO) biological activity.

7. The method of claim 2, wherein the inhibitor of the Aβ oligomer (AβO) biological activity comprises a small molecule inhibitor, an inhibitory nucleic acid, a calcium chelator, or any combination thereof.

8. The method of claim 7, wherein the small molecule inhibitor is selected from the group consisting of 3,5-dimethyladamantan-1-amine, (1S,9R)-1-methyl-16-azatetracyclo[7.6.1.0^{2,7}.0^{10,15}]hexadeca-2,4,6,10,12,14-hexaene (MK-801), (1S)-1-phenyl-2-pyridin-2-ylethanamine (lanicemine), ketamine, metabolic precursors thereof, biologically active metabolic products thereof, derivatives thereof, and pharmaceutically acceptable salts thereof, optionally wherein the derivative is nitromemantine, or any combination thereof.

9. The method of claim 7, wherein the inhibitory nucleic acid targets a nucleic acid encoding a component of an N-methyl-D-aspartate receptor (NMDAR), optionally wherein the component of the NMDAR is an NR1 gene product.

10. The method of claim 9, wherein the inhibitory nucleic acid targets a human NR1 gene product, optionally wherein the human NR1 gene product comprises any one of SEQ ID NOs: 1-12.

11. The method of claim 9, wherein the inhibitory nucleic acid targets a human NR2 gene product, optionally wherein the human NR2 gene product comprises any one of SEQ ID NOs: 19-25.

12. The method of claim 2, wherein the subject is pre-symptomatic for AD.

13. The method of claim 12, wherein the inhibitor of an Aβ oligomer (AβO) biological activity comprises a small molecule inhibitor, an inhibitory nucleic acid, a calcium chelator, or any combination thereof.

14. The method of claim 13, wherein the small molecule inhibitor is selected from the group consisting of 3,5-dimethyladamantan-1-amine, (1S,9R)-1-methyl-16-azatetracyclo[7.6.1.0^{2,7}.0^{10,15}]hexadeca-2,4,6,10,12,14-hexaene (MK-801), (1S)-1-phenyl-2-pyridin-2-ylethanamine (lanicemine), ketamine, metabolic precursors thereof, biologically active metabolic products thereof, derivatives thereof, and pharmaceutically acceptable salts thereof, optionally wherein the derivative is nitromemantine, or any combination thereof.

15. The method of claim 13, wherein the inhibitory nucleic acid targets a nucleic acid encoding a component of an N-methyl-D-aspartate receptor (NMDAR), optionally wherein the component of the NMDAR is an NR1 gene product.

16. The method of claim 15, wherein the inhibitory nucleic acid targets a human NR1 gene product, optionally wherein the human NR1 gene product comprises any one of SEQ ID NOs: 1-12.

17. The method of claim 13, wherein the inhibitory nucleic acid targets a human NR2 gene product, optionally wherein the human NR2 gene product comprises any one of SEQ ID NOs: 19-25.

18. The method of claim 2, wherein the subject is APOE4-positive.

19. (canceled)


SEQUENCE LISTING (25 sequences)

SEQ ID NO: 1 - DNA, 4379 bp, Homo sapiens
gcagtcgccgcagcgtccggaccggaaccagcgccgtccgcggagccgccgccgccgccgccgggccctttccaagccgggcgctcggagctgtgcccggccccgcttcagcaccgcggacagcgccggccgcgtggggctgagccccgagcccccgcgcacgcttcagcgccccttccctcggccgacgtcccgggaccgccgctccgggggagacgtggcgtccgcagcccgcggggccgggcgagcgcaggacggcccggaagccccgcgggggatgcgccgagggccccgcgttcgcgccgcgcagagccaggcccgcggcccgagcccatgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccagacggcatcctcgggctgcagctcatcaacggcaagaacgagtcggcccacatcagcgacgccgtgggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgtgacgatccaccaggagcccttcgtgtacgtcaagcccacgctgagtgatgggacatgcaaggaggagttcacagtcaacggcgacccagtcaagaaggtgatctgcaccgggcccaacgacacgtcgccgggcagcccccgccacacggtgcctcagtgttgctacggcttttgcatcgacctgctcatcaagctggcacggaccatgaacttcacctacgaggtgcacctggtggcagatggcaagttcggcacacaggagcgggtgaacaacagcaacaagaaggagtggaatgggatgatgggcgagctgctcagcgggcaggcagacatgatcgtggcgccgctaaccataaacaacgagcgcgcgcagtacatcgagttttccaagcccttcaagtaccagggcctgactattctggtcaagaaggagattccccggagcacgctggactcgttcatgcagccgttccagagcacactgtggctgctggtggggctgtcggtgcacgtggtggccgtgatgctgtacctgctggaccgcttcagccccttcggccggttcaaggtgaacagcgaggaggaggaggaggacgcactgaccctgtcctcggccatgtggttctcctggggcgtcctgctcaactccggcatcggggaaggcgcccccagaagcttctcagcgcgcatcctgggcatggtgtgggccggctttgccatgatcatcgtggcctcctacaccgccaacctggcggccttcctggtgctggaccggccggaggagcgcatcacgggcatcaacgaccctcggctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaacaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtcccacgagaatggcttcatggaagacctggacaagacgtgggttcggtatcaggaatgtgactcgcgcagcaacgcccctgcgacccttacttttgagaacatggccggggtcttcatgctggtagctgggggcatcgtggccgggatcttcctgattttcatcgagattgcctacaagcggcacaaggatgctcgccggaagcagatgcagctggcctttgccgccgttaacgtgtggcggaagaacctgcaggatagaaagagtggtagagcagagcctgaccctaaaaagaaagccacatttagggctatcacctccaccctggcttccagcttcaagaggcgtaggtcctccaaagacacgagcaccgggggtggacgcggcgctttgcaaaaccaaaaagacacagtgctgccgcgacgcgctattgagagggaggagggccagctgcagctgtgttcccgtcatagggagagctgagactccccgcccgccctcctctgccccctcccccgcagacagacagacagacggacgggacagcggcccggcccacgcagagccccggagcaccacggggtcgggggaggagcacccccagcctcccccaggctgcgcctgcccgcccgccggttggccggctggccggtccaccccgtcccggccccgcgcgtgcccccagcgtggggctaacgggcgccttgtctgtgtatttctattttgcagcagtaccatcccactgatatcacgggcccgctcaacctctcagatccctcggtcagcaccgtggtgtgaggcccccggaggcgcccacctgcccagttagcccggccaaggacactgatgggtcctgctgctcgggaaggcctgagggaagcccacccgccccagagactgcccaccctgggcctcccgtccgtccgcccgcccaccccgctgcctggcgggcagcccctgctggaccaaggtgcggaccggagcggctgaggacggggcagagctgagtcggctgggcagggccgcagggcgctccggcagaggcagggccctggggtctctgagcagtggggagcgggggctaactggccccaggcggaggggcttggagcagagacggcagccccatccttcccgcagcaccagcctgagccacagtggggcccatggccccagctggctgggtcgcccctcctcgggcgcctgcgctcctctgcagcctgagctccaccctcccctcttcttgcggcaccgcccacccacaccccgtctgccccttgaccccacacgccggggctggccctgccctcccccacggccgtccctgacttcccagctggcagcgcctcccgccgcctcgggccgcctcctccagactcgagagggctgagcccctcctctcctcgtccggcctgcagcccagaacgggcctccccgggggtccccggacgctggctcgggactgtcttcaaccctgccctgcaccttgggcacgggagagcgccacccgcccgcccccgccctcgctccgggtgcgtgaccggcccgccaccttgtacagaaccagcactcccagggcccgagcgcgtgccttccccgtgcggcccgtgcgcagccgcgctctgcccctccgtccccagggtgcaggcgcgcaccgcccaacccccacctcccggtgtatgcagtggtgatgcctaaaggaatgtcacgca

SEQ ID NO: 2 - DNA, 4268 bp, Homo sapiens
gcagtcgccgcagcgtccggaccggaaccagcgccgtccgcggagccgccgccgccgccgccgggccctttccaagccgggcgctcggagctgtgcccggccccgcttcagcaccgcggacagcgccggccgcgtggggctgagccccgagcccccgcgcacgcttcagcgccccttccctcggccgacgtcccgggaccgccgctccgggggagacgtggcgtccgcagcccgcggggccgggcgagcgcaggacggcccggaagccccgcgggggatgcgccgagggccccgcgttcgcgccgcgcagagccaggcccgcggcccgagcccatgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccagacggcatcctcgggctgcagctcatcaacggcaagaacgagtcggcccacatcagcgacgccgtgggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgtgacgatccaccaggagcccttcgtgtacgtcaagcccacgctgagtgatgggacatgcaaggaggagttcacagtcaacggcgacccagtcaagaaggtgatctgcaccgggcccaacgacacgtcgccgggcagcccccgccacacggtgcctcagtgttgctacggcttttgcatcgacctgctcatcaagctggcacggaccatgaacttcacctacgaggtgcacctggtggcagatggcaagttcggcacacaggagcgggtgaacaacagcaacaagaaggagtggaatgggatgatgggcgagctgctcagcgggcaggcagacatgatcgtggcgccgctaaccataaacaacgagcgcgcgcagtacatcgagttttccaagcccttcaagtaccagggcctgactattctggtcaagaaggagattccccggagcacgctggactcgttcatgcagccgttccagagcacactgtggctgctggtggggctgtcggtgcacgtggtggccgtgatgctgtacctgctggaccgcttcagccccttcggccggttcaaggtgaacagcgaggaggaggaggaggacgcactgaccctgtcctcggccatgtggttctcctggggcgtcctgctcaactccggcatcggggaaggcgcccccagaagcttctcagcgcgcatcctgggcatggtgtgggccggctttgccatgatcatcgtggcctcctacaccgccaacctggcggccttcctggtgctggaccggccggaggagcgcatcacgggcatcaacgaccctcggctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaacaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtcccacgagaatggcttcatggaagacctggacaagacgtgggttcggtatcaggaatgtgactcgcgcagcaacgcccctgcgacccttacttttgagaacatggccggggtcttcatgctggtagctgggggcatcgtggccgggatcttcctgattttcatcgagattgcctacaagcggcacaaggatgctcgccggaagcagatgcagctggcctttgccgccgttaacgtgtggcggaagaacctgcagagcaccgggggtggacgcggcgctttgcaaaaccaaaaagacacagtgctgccgcgacgcgctattgagagggaggagggccagctgcagctgtgttcccgtcatagggagagctgagactccccgcccgccctcctctgccccctcccccgcagacagacagacagacggacgggacagcggcccggcccacgcagagccccggagcaccacggggtcgggggaggagcacccccagcctcccccaggctgcgcctgcccgcccgccggttggccggctggccggtccaccccgtcccggccccgcgcgtgcccccagcgtggggctaacgggcgccttgtctgtgtatttctattttgcagcagtaccatcccactgatatcacgggcccgctcaacctctcagatccctcggtcagcaccgtggtgtgaggcccccggaggcgcccacctgcccagttagcccggccaaggacactgatgggtcctgctgctcgggaaggcctgagggaagcccacccgccccagagactgcccaccctgggcctcccgtccgtccgcccgcccaccccgctgcctggcgggcagcccctgctggaccaaggtgcggaccggagcggctgaggacggggcagagctgagtcggctgggcagggccgcagggcgctccggcagaggcagggccctggggtctctgagcagtggggagcgggggctaactggccccaggcggaggggcttggagcagagacggcagccccatccttcccgcagcaccagcctgagccacagtggggcccatggccccagctggctgggtcgcccctcctcgggcgcctgcgctcctctgcagcctgagctccaccctcccctcttcttgcggcaccgcccacccacaccccgtctgccccttgaccccacacgccggggctggccctgccctcccccacggccgtccctgacttcccagctggcagcgcctcccgccgcctcgggccgcctcctccagactcgagagggctgagcccctcctctcctcgtccggcctgcagcccagaacgggcctccccgggggtccccggacgctggctcgggactgtcttcaaccctgccctgcaccttgggcacgggagagcgccacccgcccgcccccgccctcgctccgggtgcgtgaccggcccgccaccttgtacagaaccagcactcccagggcccgagcgcgtgccttccccgtgcggcccgtgcgcagccgcgctctgcccctccgtccccagggtgcaggcgcgcaccgcccaacccccacctcccggtgtatgcagtggtgatgcctaaaggaatgtcacgca

SEQ ID NO: 3 - DNA, 4079 bp, Homo sapiens
gcagtcgccgcagcgtccggaccggaaccagcgccgtccgcggagccgccgccgccgccgccgggccctttccaagccgggcgctcggagctgtgcccggccccgcttcagcaccgcggacagcgccggccgcgtggggctgagccccgagcccccgcgcacgcttcagcgccccttccctcggccgacgtcccgggaccgccgctccgggggagacgtggcgtccgcagcccgcggggccgggcgagcgcaggacggcccggaagccccgcgggggatgcgccgagggccccgcgttcgcgccgcgcagagccaggcccgcggcccgagcccatgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaagagtaaaaaaaggaactatgaaaacctcgaccaactgtcctatgacaacaagcgcggacccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccagacggcatcctcgggctgcagctcatcaacggcaagaacgagtcggcccacatcagcgacgccgtgggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgtgacgatccaccaggagcccttcgtgtacgtcaagcccacgctgagtgatgggacatgcaaggaggagttcacagtcaacggcgacccagtcaagaaggtgatctgcaccgggcccaacgacacgtcgccgggcagcccccgccacacggtgcctcagtgttgctacggcttttgcatcgacctgctcatcaagctggcacggaccatgaacttcacctacgaggtgcacctggtggcagatggcaagttcggcacacaggagcgggtgaacaacagcaacaagaaggagtggaatgggatgatgggcgagctgctcagcgggcaggcagacatgatcgtggcgccgctaaccataaacaacgagcgcgcgcagtacatcgagttttccaagcccttcaagtaccagggcctgactattctggtcaagaaggagattccccggagcacgctggactcgttcatgcagccgttccagagcacactgtggctgctggtggggctgtcggtgcacgtggtggccgtgatgctgtacctgctggaccgcttcagccccttcggccggttcaaggtgaacagcgaggaggaggaggaggacgcactgaccctgtcctcggccatgtggttctcctggggcgtcctgctcaactccggcatcggggaaggcgcccccagaagcttctcagcgcgcatcctgggcatggtgtgggccggctttgccatgatcatcgtggcctcctacaccgccaacctggcggccttcctggtgctggaccggccggaggagcgcatcacgggcatcaacgaccctcggctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaacaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtcccacgagaatggcttcatggaagacctggacaagacgtgggttcggtatcaggaatgtgactcgcgcagcaacgcccctgcgacccttacttttgagaacatggccggggtcttcatgctggtagctgggggcatcgtggccgggatcttcctgattttcatcgagattgcctacaagcggcacaaggatgctcgccggaagcagatgcagctggcctttgccgccgttaacgtgtggcggaagaacctgcaggatagaaagagtggtagagcagagcctgaccctaaaaagaaagccacatttagggctatcacctccaccctggcttccagcttcaagaggcgtaggtcctccaaagacacgcagtaccatcccactgatatcacgggcccgctcaacctctcagatccctcggtcagcaccgtggtgtgaggcccccggaggcgcccacctgcccagttagcccggccaaggacactgatgggtcctgctgctcgggaaggcctgagggaagcccacccgccccagagactgcccaccctgggcctcccgtccgtccgcccgcccaccccgctgcctggcgggcagcccctgctggaccaaggtgcggaccggagcggctgaggacggggcagagctgagtcggctgggcagggccgcagggcgctccggcagaggcagggccctggggtctctgagcagtggggagcgggggctaactggccccaggcggaggggcttggagcagagacggcagccccatccttcccgcagcaccagcctgagccacagtggggcccatggccccagctggctgggtcgcccctcctcgggcgcctgcgctcctctgcagcctgagctccaccctcccctcttcttgcggcaccgcccacccacaccccgtctgccccttgaccccacacgccggggctggccctgccctcccccacggccgtccctgacttcccagctggcagcgcctcccgccgcctcgggccgcctcctccagactcgagagggctgagcccctcctctcctcgtccggcctgcagcccagaacgggcctccccgggggtccccggacgctggctcgggactgtcttcaaccctgccctgcaccttgggcacgggagagcgccacccgcccgcccccgccctcgctccgggtgcgtgaccggcccgccaccttgtacagaaccagcactcccagggcccgagcgcgtgccttccccgtgcggcccgtgcgcagccgcgctctgcccctccgtccccagggtgcaggcgcgcaccgcccaacccccacctcccggtgtatgcagtggtgatgcctaaaggaatgtcacgca

SEQ ID NO: 4 - DNA, 3905 bp, Homo sapiens
gcagtcgccgcagcgtccggaccggaaccagcgccgtccgcggagccgccgccgccgccgccgggccctttccaagccgggcgctcggagctgtgcccggccccgcttcagcaccgcggacagcgccggccgcgtggggctgagccccgagcccccgcgcacgcttcagcgccccttccctcggccgacgtcccgggaccgccgctccgggggagacgtggcgtccgcagcccgcggggccgggcgagcgcaggacggcccggaagccccgcgggggatgcgccgagggccccgcgttcgcgccgcgcagagccaggcccgcggcccgagcccatgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccagacggcatcctcgggctgcagctcatcaacggcaagaacgagtcggcccacatcagcgacgccgtgggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgtgacgatccaccaggagcccttcgtgtacgtcaagcccacgctgagtgatgggacatgcaaggaggagttcacagtcaacggcgacccagtcaagaaggtgatctgcaccgggcccaacgacacgtcgccgggcagcccccgccacacggtgcctcagtgttgctacggcttttgcatcgacctgctcatcaagctggcacggaccatgaacttcacctacgaggtgcacctggtggcagatggcaagttcggcacacaggagcgggtgaacaacagcaacaagaaggagtggaatgggatgatgggcgagctgctcagcgggcaggcagacatgatcgtggcgccgctaaccataaacaacgagcgcgcgcagtacatcgagttttccaagcccttcaagtaccagggcctgactattctggtcaagaaggagattccccggagcacgctggactcgttcatgcagccgttccagagcacactgtggctgctggtggggctgtcggtgcacgtggtggccgtgatgctgtacctgctggaccgcttcagccccttcggccggttcaaggtgaacagcgaggaggaggaggaggacgcactgaccctgtcctcggccatgtggttctcctggggcgtcctgctcaactccggcatcggggaaggcgcccccagaagcttctcagcgcgcatcctgggcatggtgtgggccggctttgccatgatcatcgtggcctcctacaccgccaacctggcggccttcctggtgctggaccggccggaggagcgcatcacgggcatcaacgaccctcggctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaacaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtcccacgagaatggcttcatggaagacctggacaagacgtgggttcggtatcaggaatgtgactcgcgcagcaacgcccctgcgacccttacttttgagaacatggccggggtcttcatgctggtagctgggggcatcgtggccgggatcttcctgattttcatcgagattgcctacaagcggcacaaggatgctcgccggaagcagatgcagctggcctttgccgccgttaacgtgtggcggaagaacctgcagcagtaccatcccactgatatcacgggcccgctcaacctctcagatccctcggtcagcaccgtggtgtgaggcccccggaggcgcccacctgcccagttagcccggccaaggacactgatgggtcctgctgctcgggaaggcctgagggaagcccacccgccccagagactgcccaccctgggcctcccgtccgtccgcccgcccaccccgctgcctggcgggcagcccctgctggaccaaggtgcggaccggagcggctgaggacggggcagagctgagtcggctgggcagggccgcagggcgctccggcagaggcagggccctggggtctctgagcagtggggagcgggggctaactggccccaggcggaggggcttggagcagagacggcagccccatccttcccgcagcaccagcctgagccacagtggggcccatggccccagctggctgggtcgcccctcctcgggcgcctgcgctcctctgcagcctgagctccaccctcccctcttcttgcggcaccgcccacccacaccccgtctgccccttgaccccacacgccggggctggccctgccctcccccacggccgtccctgacttcccagctggcagcgcctcccgccgcctcgggccgcctcctccagactcgagagggctgagcccctcctctcctcgtccggcctgcagcccagaacgggcctccccgggggtccccggacgctggctcgggactgtcttcaaccctgccctgcaccttgggcacgggagagcgccacccgcccgcccccgccctcgctccgggtgcgtgaccggcccgccaccttgtacagaaccagcactcccagggcccgagcgcgtgccttccccgtgcggcccgtgcgcagccgcgctctgcccctccgtccccagggtgcaggcgcgcaccgcccaacccccacctcccggtgtatgcagtggtgatgcctaaaggaatgtcacgca

SEQ ID NO: 5 - DNA, 3968 bp, Homo sapiens
gcagtcgccgcagcgtccggaccggaaccagcgccgtccgcggagccgccgccgccgccgccgggccctttccaagccgggcgctcggagctgtgcccggccccgcttcagcaccgcggacagcgccggccgcgtggggctgagccccgagcccccgcgcacgcttcagcgccccttccctcggccgacgtcccgggaccgccgctccgggggagacgtggcgtccgcagcccgcggggccgggcgagcgcaggacggcccggaagccccgcgggggatgcgccgagggccccgcgttcgcgccgcgcagagccaggcccgcggcccgagcccatgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaagagtaaaaaaaggaactatgaaaacctcgaccaactgtcctatgacaacaagcgcggacccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccagacggcatcctcgggctgcagctcatcaacggcaagaacgagtcggcccacatcagcgacgccgtgggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgtgacgatccaccaggagcccttcgtgtacgtcaagcccacgctgagtgatgggacatgcaaggaggagttcacagtcaacggcgacccagtcaagaaggtgatctgcaccgggcccaacgacacgtcgccgggcagcccccgccacacggtgcctcagtgttgctacggcttttgcatcgacctgctcatcaagctggcacggaccatgaacttcacctacgaggtgcacctggtggcagatggcaagttcggcacacaggagcgggtgaacaacagcaacaagaaggagtggaatgggatgatgggcgagctgctcagcgggcaggcagacatgatcgtggcgccgctaaccataaacaacgagcgcgcgcagtacatcgagttttccaagcccttcaagtaccagggcctgactattctggtcaagaaggagattccccggagcacgctggactcgttcatgcagccgttccagagcacactgtggctgctggtggggctgtcggtgcacgtggtggccgtgatgctgtacctgctggaccgcttcagccccttcggccggttcaaggtgaacagcgaggaggaggaggaggacgcactgaccctgtcctcggccatgtggttctcctggggcgtcctgctcaactccggcatcggggaaggcgcccccagaagcttctcagcgcgcatcctgggcatggtgtgggccggctttgccatgatcatcgtggcctcctacaccgccaacctggcggccttcctggtgctggaccggccggaggagcgcatcacgggcatcaacgaccctcggctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaacaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtcccacgagaatggcttcatggaagacctggacaagacgtgggttcggtatcaggaatgtgactcgcgcagcaacgcccctgcgacccttacttttgagaacatggccggggtcttcatgctggtagctgggggcatcgtggccgggatcttcctgattttcatcgagattgcctacaagcggcacaaggatgctcgccggaagcagatgcagctggcctttgccgccgttaacgtgtggcggaagaacctgcagcagtaccatcccactgatatcacgggcccgctcaacctctcagatccctcggtcagcaccgtggtgtgaggcccccggaggcgcccacctgcccagttagcccggccaaggacactgatgggtcctgctgctcgggaaggcctgagggaagcccacccgccccagagactgcccaccctgggcctcccgtccgtccgcccgcccaccccgctgcctggcgggcagcccctgctggaccaaggtgcggaccggagcggctgaggacggggcagagctgagtcggctgggcagggccgcagggcgctccggcagaggcagggccctggggtctctgagcagtggggagcgggggctaactggccccaggcggaggggcttggagcagagacggcagccccatccttcccgcagcaccagcctgagccacagtggggcccatggccccagctggctgggtcgcccctcctcgggcgcctgcgctcctctgcagcctgagctccaccctcccctcttcttgcggcaccgcccacccacaccccgtctgccccttgaccccacacgccggggctggccctgccctcccccacggccgtccctgacttcccagctggcagcgcctcccgccgcctcgggccgcctcctccagactcgagagggctgagcccctcctctcctcgtccggcctgcagcccagaacgggcctccccgggggtccccggacgctggctcgggactgtcttcaaccctgccctgcaccttgggcacgggagagcgccacccgcccgcccccgccctcgctccgggtgcgtgaccggcccgccaccttgtacagaaccagcactcccagggcccgagcgcgtgccttccccgtgcggcccgtgcgcagccgcgctctgcccctccgtccccagggtgcaggcgcgcaccgcccaacccccacctcccggtgtatgcagtggtgatgcctaaaggaatgtcacgca

SEQ ID NO: 6 - DNA, 471 bp, Homo sapiens
atcaacggcaagaacgagtcggcccacatcagcgacgccgtgggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgtgacgatccaccaggagcccttcgtgtacgtcaagcccacgctgagtgatgggacatgcaaggaggagttcacagtcaacggc

SEQ ID NO: 7 - DNA, 20 bp, Homo sapiens
cttctaccgcatacccgtgc

SEQ ID NO: 8 - DNA, 20 bp, Homo sapiens
gcttaccttgtccgagtaga

SEQ ID NO: 9 - DNA, 20 bp, Homo sapiens
gccgtcacgttcttggtccc

SEQ ID NO: 10 - DNA, 20 bp, Homo sapiens
ccgggcggctcagaaacgcc

SEQ ID NO: 11 - DNA, 20 bp, Homo sapiens
catcatctcaaaccacacgc

SEQ ID NO: 12 - DNA, 20 bp, Homo sapiens
gaacgtgacggccctgctga

SEQ ID NO: 13 - DNA, 20 bp, Homo sapiens
gcacggtcccaaagcggaaa

SEQ ID NO: 14 - DNA, 20 bp, Homo sapiens
cagttccgacattgctttat

SEQ ID NO: 15 - DNA, 20 bp, Homo sapiens
tgcagcagtgctgaactata

SEQ ID NO: 16 - DNA, 20 bp, Homo sapiens
atgcatcatctacacccctc

SEQ ID NO: 17 - DNA, 20 bp, Homo sapiens
caggggtcttctacatgttg

SEQ ID NO: 18 - DNA, 20 bp, Homo sapiens
cactgctgcatcatagatga

SEQ ID NO: 19 - DNA, 30609 bp, Homo sapiens
acttcctccggcttcttcacctctaggaaatctcggggcttcgctctatcgagagtgctcgtcaagggtgattcttgatcaccaccatccagtagtgctggcgtccgaatgcatatccttttgccttgcaaagaagagtcatgaacttcaaaacccccgagacggatcattaatcctgctttgcaggaaaggaaaaccctccattcccagccaccggccacccctctccgcagactgcaaccgcggaggcgctcggagccggaaggggacgctttgggaatgaccatgctccaccgagggacggagccggcccccagcttctccacacagagcctcctccactaacgctccaaaaaccaaaaaccgtaattgccagaagaagcgttaaaaatctattccagccactaacctcacatgcacacggaataattactctggattctgcattgtgagctgctctccataccctgaattacctttgaattaaatctttttttttttgaatttgcatctcttcaagacacaagattaaaacaaaatttacgctaaattggattttaaattatcttccgttcatttatccttcgtctttcttatgtggatatgcaagcgagaagaagggactggacattcccaacatgctcactcccttaatctgtccgtctagaggtttggcttctacaaaccaagggagtcgacgagttgaagatgaagcccagagcggagtgctgttctcccaagttctggttggtgttggccgtcctggccgtgtcaggcagcagagctcgttctcagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcacccccatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcaggaggatgaccatctgagcattgtgaccctggaggaggcaccatttgtcattgtggaaagtgtggaccctctgagtggaacctgcatgaggaacacagtcccctgccaaaaacgcatagtcactgagaataaaacagacgaggagccgggttacatcaaaaaatgctgcaaggggttctgtattgacatccttaagaaaatttctaaatctgtgaagttcacctatgacctttacctggttaccaatggcaagcatgggaagaaaatcaatggaacctggaatggtatgattggagaggtggtcatgaagagggcctacatggcagtgggctcactcaccatcaatgaggaacgatcggaggtggtcgacttctctgtgcccttcatagagacaggcatcagtgtcatggtgtcacgcagcaatgggactgtctcaccttctgccttcttagagccattcagcgctgacgtatgggtgatgatgtttgtgatgctgctcatcgtctcagccgtggctgtctttgtctttgagtacttcagccctgtgggttataacaggtgcctcgctgatggcagagagcctggtggaccctctttcaccatcggcaaagctatttggttgctctggggtctggtgtttaacaactccgtacctgtgcagaacccaaaggggaccacctccaagatcatggtgtcagtgtgggccttctttgctgtcatcttcctggccagctacactgccaacttagctgccttcatgatccaagaggaatatgtggaccaggtttctggcctgagcgacaaaaagttccagagacctaatgacttctcaccccctttccgctttgggaccgtgcccaacggcagcacagagagaaatattcgcaataactatgcagaaatgcatgcctacatgggaaagttcaaccagaggggtgtagatgatgcattgctctccctgaaaacagggaaactggatgccttcatctatgatgcagcagtgctgaactatatggcaggcagagatgaaggctgcaagctggtgaccattggcagtgggaaggtctttgcttccactggctatggcattgccatccaaaaagattctgggtggaagcgccaggtggaccttgctatcctgcagctctttggagatggggagatggaagaactggaagctctctggctcactggcatttgtcacaatgagaagaatgaggtcatgagcagccagctggacattgacaacatggcaggggtcttctacatgttgggggcggccatggctctcagcctcatcaccttcatctgcgaacaccttttctattggcagttccgacattgctttatgggtgtctgttctggcaagcctggcatggtcttctccatcagcagaggtatctacagctgcatccatggggtggcgatcgaggagcgccagtctgtaatgaactcccccaccgcaaccatgaacaacacacactccaacatcctgcgcctgctgcgcacggccaagaacatggctaacctgtctggtgtgaatggctcaccgcagagcgccctggacttcatccgacgggagtcatccgtctatgacatctcagagcaccgccgcagcttcacgcattctgactgcaaatcctacaacaacccgccctgtgaggagaacctcttcagtgactacatcagtgaggtagagagaacgttcgggaacctgcagctgaaggacagcaacgtgtaccaagatcactaccaccatcaccaccggccccatagtattggcagtgccagctccatcgatgggctctacgactgtgacaacccacccttcaccacccagtccaggtccatcagcaagaagcccctggacatcggcctcccctcctccaagcacagccagctcagtgacctgtacggcaaattctccttcaagagcgaccgctacagtggccacgacgacttgatccgctccgatgtctctgacatctcaacccacaccgtcacctatgggaacatcgagggcaatgccgccaagaggcgtaagcagcaatataaggacagcctgaagaagcggcctgcctcggccaagtcccgcagggagtttgacgagatcgagctggcctaccgtcgccgaccgccccgctcccctgaccacaagcgctacttcagggacaaggaagggctacgggacttctacctggaccagttccgaacaaaggagaactcaccccactgggagcacgtagacctgaccgacatctacaaggagcggagtgatgactttaagcgcgactccgtcagcggaggagggccctgtaccaacaggtctcacatcaagcacgggacgggcgacaaacacggcgtggtcagcggggtacctgcaccttgggagaagaacctgaccaacgtggagtgggaggaccggtccgggggcaacttctgccgcagctgtccctccaagctgcacaactactccacgacggtgacgggtcagaactcgggcaggcaggcgtgcatccggtgtgaggcttgcaagaaagcaggcaacctgtatgacatcagtgaggacaactccctgcaggaactggaccagccggctgccccagtggcggtgacgtcaaacgcctccaccactaagtaccctcagagcccgactaattccaaggcccagaagaagaaccggaacaaactgcgccggcagcactcctacgacaccttcgtggacctgcagaaggaagaagccgccctggccccgcgcagcgtaagcctgaaagacaagggccgattcatggatgggagcccctacgcccacatgtttgagatgtcagctggcgagagcacctttgccaacaacaagtcctcagtgcccactgccggacatcaccaccacaacaaccccggcggcgggtacatgctcagcaagtcgctctaccctgaccgggtcacgcaaaaccctttcatccccacttttggggacgaccagtgcttgctccatggcagcaaatcctacttcttcaggcagcccacggtggcgggggcgtcgaaagccaggccggacttccgggcccttgtcaccaacaagccggtggtctcggcccttcatggggccgtgccagcccgtttccagaaggacatctgtatagggaaccagtccaacccctgtgtgcctaacaacaaaaaccccagggctttcaatggctccagcaatgggcatgtttatgagaaactttctagtattgagtctgatgtctgagtgagggaacagagaggttaaggtgggtacgggagggtaaggctgtgggtcgcgtgatgcgcatgtcacggagggtgacgggggtgaacttggttcccatttgctcctttcttgttttaatttatttatgggatcctggagttctggttcctactgggggcaaccctggtgaccagcaccatctctcctccttttcacagttctctccttcttccccccgctgtcagccattcctgttcccatgagatgatgccatgggccctctcagcaggggagggtagagcggagaaaggaagggctgcatgcgggcttcctcctggtgtggaagagctccttgatatcctctttgagtgaagctgggagaaccaaaaagaggctatgtgagcacaaaggtagcttttcccaaactgatcttttcatttaggtgaggaagcaaaagcatctatgtgagaccatttagcacactgcttgtgaaaggaaagaggctctggctaaattcatgctgcttagatgacatctgtctaggaatcatgtgccaagcagaggttgggaggccatttgtgtttatatataagccaaaaaatgcttgcttcaaccccatgagactcgatagtggtggtgaacagaacaaaaggtcattggtggcagagtggattcttgaacaaactggaaagtacgttatgatagtgtcccacggtgccttggggacaagagcaggtggattgtgcgtgcatgtgtgttcatgcacacttgcacccatgtgtagtcaggtgcctcaagagaaggcaaccttgactctttctattgtttctttcaatatccccaagcagtgtgattgtttggcttatatacagacagagatggccatgtattacctgaattttggctgtgtctcccttcatccttctggaataaggagaatgaaaattcttgataaagaagattctgtggtctaaacaaaaaaaggcggtgagcaatcctgcaagaacaaggtacataaacaagtcctcagtggttggcaattgtttcaaccagtttgaaccaagaactttccaggaaggctaaagggaaaccgaattttcacagccatgattcttttgcccacacttgggagcaaaagattctacaaagctcttttgagcatttagactctcgactggccaaggtttggggaagaacgaagccacctttgaagaagtaaggagtcgtgtatggtagggtaagtgagagagggggatgtttccaatgctttgatcccttcttacttaacctgaagctagacgagcaggcttcttccccccaaaactgattacaactgctacagagcagacagttaagagaaatgagcttgacctttaagagaaatgagctgcactccatgagtgcagctctggaggtacgaaaagaggggaagagacttggaaatgggagacgggggcagagagggaccctccaccacctctttgggcctggctccctgggaatgtgacttgagcccagagtgaacactcttggtagaagcccttctaccttcctgcaacaccttgtttccctctcagattgtaccattgaggaataaaaaaagatatacatgtataaaactccagggatgagggtaccagcagaactgatgctcccttcacttaacattggaaaattgagaagggaaatgaatatgtatcactgccggtcctgaaacttgcggtagggctatttattctatgggtcagttccactgtcctgccatcctcccctacacagggtggcatccagagagccccaggtctgtgtggaagcttgactgcaggatcgccatgttgcacacaggacacataaaccagaaaggtgctcatcctcctctggaaggaagctgccatgtctcttcaagtcactccctccagggctgctcttcgcccctgtttgccctcactcgtgacccttcctcatcccgcccttgactgctgtccccaaccctcctccttgatttttacctcagtatctttcattttaaagccaactgaaggccttgtaaagctttgttttctttctaaaatgaacatgggggcaaggggctgaacctctcacagatttggtgagaggagcacggagtgctttaccttagataacagaaaagactgagagaggacagagggcatgaggcacctccaggaggaagaaggcgaagggccacgtaaacactgcttcgccctgactctgtccaggggtcagggactcatcccctcatcttcctcttacctcacccctttcaaacaaaaaaaccaaaccaactaacctagcgaacagcaatctagaaccaagggagctctttcagtctccgctggcaacagaaatcaagatggtgtttatttcatatgtaaatattttgttttctaattttgtatagataaagtgttctcttgtgagtattcagggtgttgggctggagggagtggggtggtgatgggaatgagagggaaaaagtgttttgttatgggttttcatttcccattgggggaattgttttgtatggcttttttgcttcagggtgtctggaaaaagcgaatgctcatggaggccactatggtgaaaaagccaggggaaagtaattgtgtccatcccccccagaaaaccagtcttcttactggtttgtgagaaatgttcatgcactttggccagggttttacatagggttcaatttggctctaagcaaaccccaggtctgactcctatcttctaggtagaccctaggttaccacattccacacaaagacaggctggtggcaccatatttcctctgtcagtacctagggtaggaaagattcccaaaagggcttaaacagaattaaggtcaaattaacttcccattcttggcgtgactgaaaggtttcaggaagctcaatcctgagaaatctcccagcggcctcctctgatgctgaaacaatcgtctcagtcatctgagtcttttcttctttaggacctgggcgctgactcactccctgccccatctccaccctcagaagtcagcctggcaaacacacgtggaaaaatctccactaactctactttttgaagcatgtgatatctggtcacagagaggtagtgctggatatgtgtcttacaaatttgcatgcttttctagaaaagatgatgggatccactagaaactcagtggtgagatgattcccttagcaagattgctgaagttaggtttagacgtgggagggtgggtatgtgagcaatggtgccaatagcggctctttatttgccttgtcctcattactgccatcaggaaggtgctactggcctcgagccaggtgttcataatctggccttgggttaaccagacaaatagaacttcttttcctagactgttggctttgtggaggttggcagcctctatcacaggataaaatttccaaatccatttacccagtatattcactacattttttcctatcctcccatcataaaagctgttagctggacaattttatttttctaaacttatttggccccagttggttgcttttcttcaactggatatttgcactgggactaagttactcatggtctgcttaagaaaacttaaagaaaggaaggctgggcattgttaaaatctagatgttacttaattccagaataacaagcaagtaaaatgcatgaggaagaattcagctataagcccaatctatatcacagatgggtatgaaacaacacccaatccatccaaaactgtgagattccgcagataccctcaggagcacctaatattcccatgttcttcagaatccatgctttctcgctttgtaccctctatcattcccttccattgcagaagtggttggtgagtgtggtcctgtatgaactcctttcagtggcaatgcttaaatctcagcatttccactgacaataatctgacccttgaatttgcctgagtcgagactttgcacacccttcaaagtgatgcatgtacagtggactttgttatttcaaaatctgtcactgtgtatcctcagccagaacttgggaaacatccttcatatgtatttccctgcactggccacagccatgttgtgattgaggggaaaatcccaaatgattccattaaatgttgctggtgtcagtagtgccaaaaacgcatgtatgacatgatacagtaggagttgtctatacagttaagtgactatcactcacccggtcttccgtggccatcaggagttgcccaagggagaattctctgcagagtgctggtcacctgcacatgtcactaagaactaggtttctttggttcattttttttttttttttggtcggtttttagttagttagttcaagatttacagatagaataaccagattgtttgggcaataagcttttggaaaaactattccagaactcactttattctttcaatcatcatctctctaaaaaggccctgtcattgcttgacaccctgtgaatgtctgtttttgtatgttaggaatgattcagatgtgtgcataaatgatgtcactgcagtgatattgatgaatgcattcagcatttccagggactgtggccataccttatgtgctctctgagcccaaagccatataggaaatacatgctcagtaggctgctacacactacagcagagcttttgctttcttcacttgggtgtcagagacttttcctcctgtttaaggagctcttcatggaggagaagcagctgaaagtgaatagtcacactgggatgggtctggccttatgtatgctatgtggtccatggtgtgttgaaggtcctgaaaaggtctttttcatttcccaccccactgcaactaccaccttcccacacagatttgttgctttagcactgagctgagctgtctgtcattcgaatgaaagtaatcccaggaaaggtgacatattaagccatattagggatgtatacatttaatcacctaatttacattaggaatctcactccttttcctgggaataagttatgatacaagagaagaggaagggagcagagagcactgagagcaatagagtagggaacaaggatacaggttctagatggcaaagacatttaaaccatattttggcctccgcctgcttttatttaagcctcatttgacacctaaataggactcctggttaggaattattttcaatacacaatcatatgagtgtataatttatgaatttctaaattcagtatcttgtagggcaggttgagaactatgattctttccatagaaaatagaagagtgatgcttctcctccattacatgttactgagtaggcagctagaaaaatcaaattgaattagtgatacaataaaatttactgtcattgtctggtttctgtcccagtttaattaatcttccaattagctgagttggtctatatcctatactttccacctccatagggggtgccctgttatctggtaaaagagccacttatgacctcaggtgctacttaacctggggggcaattgtttcttaggcctagcagatgtttgggatgacactaaaatggcagattagtcagtctgagaatgtctaatattgatctgaatctaggattctattctagtttggggggtggggttgcagtattgtataaaatcataggatatctgagaacatcccaccctcaactcattccaaccacattgattcagtcacacagtggccctgtgagctctatgacttagaagagatagcctatctattcctatgacaaaggccctcacccaaaaccttaaattgaaagccagtgctgctactttcatgccttggagagaactgtgacgtttagggcttctcatttacaatattgaagcaagaaaaaagaattttgttcagagtaatgggttagaatagcagtactgggaataagtagggaaagttagaaatcttccacatttctctctacatctctcaagagctctgtacagtgagtttctggagctgcttcatgctttgaatataattgctggggtgattatgtggttaagggctcctgaaagaactgaaggtgaaaagcagagtagatgagtagaattcctcagagccttggcactctggtgtgtagaatctgtgttttggagaaggtgaaagaactcagtgcaagaattaaggtacaattaaaaagcacaacactaagcccaatttaaactgttattttaaagattaggtttccacactttaaacctgaactcttttatcggtaattattttaatgttgtgatttaggaggtggagcagattctagtactatgcaggagaaaaaaataaaagctctctggttaaatatgtgagtttaattaaaccaacgtctattgtgcacgtactatgtccctaggctagttctgtgaatgatataaaaggtttcaaaatggagtatattgtactcacgtagaatatatgaaagcataagtataaaatatgtaaatgtatgtataaataggtatgcatacatttgtatgattatacatgcaagtttaatattttctggtttcagggtaggagtcaaagatatccattatgctatttgaaaaaacattgcagagattcagcttcccatgtcatggccaggcctggacttattcttttgaaattattacacctcccagagctaaaactctgggatttcctctctaaccacaaccttgccttttctatatcttccttccttcaagacccctctgttgttctggtccataaatccatcaggcctcattgaattccctctttggtctaaccctgtatttacccacattagcaatattctcaccggcaattcctttttcctcttgccatctcactacaccaaccatgcttagctaactgcatcccatccccttgtcattcattgcatccccaagaagagcaagtcttaaacctacgtgaagagcacaaattcaatcaatttaacctcacctgaagcctactcacttggactttttaatttctccaatgttgactcagtgtcacattctgtgcttgataaatttgttttattttcctttcatgttcatgtcctgtctttcaacaggcaatctttcctactgtgtttgcaagaaaatctgattattgaatcctcacatatgctgtcctctctgcctttagatttctcttgtctttcacacatcttttccatcctttctcccaaccaagtgctcctcttccctatgctctgacctcttctgaggtgctctccagccaattgtatttctctcttttgcatctttaattccttccacacatctacctgcaactatctcaaaaacacccgtcctacaactatctctaaagacaaatctctcaatccaatttgactcttgaatttctgtcctatcgctatcttttcacttcccaacttctcaaaagggtagtctttctttctccacttcctcaccttatgctcatggcttcatcaatgggctacaccctaccactcttctgaaactactctcaggcaacgttccaatgaagttcttgttacaaaagtcaatagccttttttctttacttaccctgctcttcaacctcattgacaattaccattgggaggcaatacattctggttttaatcttagttctctggtcctctaacctcattggttcctgctcttcctcttatctccttagcattgacttgccatggtatatctgagcttgaccctcttctccttatcttaatgcttctgtagcaatctttctgtcttctatgatttcaataactctttgaatgaagatgaatcccaaatcttcacttttagcccctaccttttgcttttccttcatacacatacttccagtcaccttctgaacactcaacttcatcttgactcaaatatccaaaaccattgctgcaaaccaacataatcaccaacttcctccttttgcttaatggtatcaccatgtcttccgtcaccgaactcaagcttttcaggcacttttgcctcctccattttcttcatcctgcatatctaaaaatttgaaaagcctactaattcttccccctaaggtttccagtctttccttttcattcagacccttccttctagtctggattatttcagcgtcttttagcattgttccaatattttatatttgaatctactcttgtcagtactcacttcctacatataaatcagatgtcattcttcagatcaagaaacaaccttcagtgggcttcacttgccgtagtctgccagcaataatttcttgttacttgactatgtgtgtgtcccataatccagacaagctgaataacatttgtttcacctttccctaccagcttgttttcccacttgcgtcttcccttaaactgttccctttgtccaaaatactctccttcctatctcccatctcactcccttcttgaataatatccattccctctatataagtcttcaaagaccagaacaacttctttatcttccattcagaaaataattttaggcattttatgaatgcccataacactttcgttgtttctccctagtggcagctatcatgggccatcttatttcataggtatttggttatacatctcatctctcctgctacagcataagctccctgtgttcagggccatgtatttttaaatctttttatctcctgtagcactaaacagagtgccttgtatatgaaaggcattagtaaatatttgatattattataaattataattgagaaggagtcactttattttgcccatttgtatctgcagtagcacctaaggatgaatccccccagtttctctatatatatctgcacacacaatcacatgtatatactacatgtatacagttcattatttcaaatggaatctggatttttctaagtgctttgtcctttttctaagtgctttgtcctttgaagttgttttggactgttttatggttaggaaagtgtacttggaacactacatgtgttcaagtaagtgacagagggagaaactatgaaggatattgatcaaattttaggtatcagcttttcagatgctttgggtagagcttgtatccagacactttactaccaagattgagtctctaataacctgatccatgctgggactgatctgtgatccaatatttagcttcttttttaacactttcagcaccaaaagaaccttgtcacagttctggctcactaatcacacagccttgtagaccccaggtggtggtccaacaggaccactgtatttcaccacacgctacttccacatttgtggccttgggggaactgtctgtgctggtcaccaccttgtctttatatttctttcctgagttctttggcttctcaccctttctgaggctttgtctacaggatcacttctcccaaagggaggataagagttgaggtaattgagcactttacctcttatctgtatatttacaatgaaagtaagttaggatttgaatgagctggctcagagagaatgctagtctctaaactactcctttctctgtaaatataaagggattcattacctcagggggagacaacatcacttctgaccttatggccctcccttatcctctggtccctgtattcttgaagccctgttaacccttcctcctcctctcttggctctacttagcaatctatcaatcattctttctagccagtgtcagtctctgtccctctgaagaaactcttggagctcgtactgtcctttcacttcagctgctctggtaaccgataatgttttcgttccttctcccggccagccttctctgtaaatctcattatgccattggctatgaattattcccctttgggaggcatcttctgccactgtgtgctaatttaaagcatactatagagaactgaactcaaataagttacaggcagtgacaagtctctttttttaaaaaaaaagtgaagaaagtccactcttcattgagacaggagtgggtaggacaattattattggcaaggtagggcaaaacttgtgacaacttgggagatacctagtgaaaattaaaatataagtctagagagataattttgcttttcaaaagtcctatggggtctagatagccttgctcctgaaaatgagaatagttaatattttagagcaaaacccatttcattatactatgagacttttttttttagctgatgaaacaaattatctggtaaacagctggtgggagggtaggaagacagccatcctcatgaaggacgttcatgaatctcatttgggccaataaatggaccaaaattctaggcattttagccatgaactgttcatgtcaattattttgcctacaaatacccataaaaggaaagggaggatatgaggaaggaaaagaacaggaagtctacactggagcttccttttgtgtcaccaataaagccagaagctgacatgtgaggcacatggttcccccttggttactggcttcaatgatagcttcggaaagtagggaaacttgaaagagcaaggaccaaagcggagatgagtgaggaacctgaatgcaaccttatgtgtgaggtcctgatatccacaaggaacctggccccatgtcattcctgcccaggtccttcagattacagcacccaaggagcccttgatcatcaccctgggcagagggagtgacttatcacaccacccttacccacagaatttaggccaccaggctgtgactccatctgtcccccttgcagtaagagattcatagtctctgaaatctgacaggagaacagcatcaggggaaggggccacaggaatgcgaatttgttgagctgcctgctcctctaggataagtatattacctgtttccagaggggattgtttttttttcgaaatgaaacaaaacatgcctgcctaaaacttcagccctattcagaacagtcttgcagttaactgtcattgtcacattttaattgtaattgattttcttttgaaatccatggctattttaatgctccctttgaccatgacataagaaccctctgcaaaccagagacctggccattaggttgcctctggctgagttatactctcagtagtttatactttgctctcagatattcacctgaacagcacaactacacagaatttgatctctttgccatgagcattctgatgcctatgtacctccctaatgcacacacacaatcttaacaaatagatccaaatttcctatgtcaatcatgttatggtagatgaaagtgttcatttagaatgggataaagagacatatcataatttatgcagccagtgttttctctttgctctacctggcttgtggatcattgatccctcaaggtttgacagaaatattcaggcttcatgtgaaggcagcatgttctttgtgatgaaacaacctagttatcgtctccattttcccattttaccctatggaatccatgtggccttctaaggtcctgttgatggactggtactaagaggatggagaagcagaagccagtccccctgagaacagacagtcctgttgcttagatacatacatattcgcacatgtgcccacatctcttcttggaaacatcatgagcagaggtggagaggtctgttctcaatgggaggaataagggtaggagaattccttgagaatccctattctcaaggaatagggataaatgccttcacggattcaataatttatctgttctctctctcccagggtcaggagcaagaaaaatatcaaggtttcacataggcaggaagcaaggaggatagtgaagcatctaggtggaggtggaactcttactgagaaaaacatgaagtagtaaaatcaccctttctttgatataaatgcatttagaagtcaatgagggcacttacagctgtgtttcccttcactaacttttcatctcttgcattctttacttctctttctcaatttattcatcttgaggtatacttatcctgagttgactctgagcagcccatcctcaaaacacaaacaatgttaggatgagaagttgagtgagcacagagctcgaacacgtgcatggtacttaatgtttccaaactcgagcccaatcagtctagcacagtcaatcccactgcattttaatgactaggatggggcctgctgtacatagctggacatcttggattacagaaaagaggctttatgtgaaggcagcattttctttgtggtagagcaacatagttatcctgctaactgtcctatttgacctgatggaatccataaggtcctgttgatgggttggttctaagaggatggagaagcagaagtcattctactgagaacagacagtcctgttgcttagatacatacatattctttatgacagaattcctcctgagactcttgcacaccttcagtgatcagcgcattcactttggttatttcttggtaacaggcacgagaaatgggtcacatagaaatctgaagagtcatttctcgagcatttttagcttattcccatgtaaagaggcactgagctcctgcagttctagcctgcctctgctcccctccttggggaatggtggagtttctaggataaccagcaacccattaggctccaccacacttagcctatggtacattctgagtaaatcacagtagttggtatgtgaatacagaagaatttaccataaccacttctccaaataacctcctttgacttggaaataagatcatctttcagttttacagatgagaagtcaaagcacaaaattggataattagccaaaagtaagattatacagaaaattcttgacagaaccaaaacttggctgaaccaaaacttgtccaaatttctaactctcagtctagctcagcgagtaatgctgcctgaattgtctgcccccactgtaggttttcagcatctagcagtggagaaaagtcacgaaatctaggcagctgatttagccagatcttatctatgaaatttccctttgtagacacacacacactccacatacatctacagagacaagtcaaaaagagcaagaagaaagcaaaaatttgaaaatcagaaaggttagagaggtttccacggtgaagaaaacttggtcaaataaaaatgtttttgaattctatagggtaagatattgtgaataattttttattattaaaaattttttttaggtagcatgtaatccacataaataaaaaactcaagaataaactgggacagaggatttcttccagagtgctacatggcaaaagagccttcaggaacgtgtgcaggggtgtcatgatactccgaaggtgtcagtagttttggtcagcccacagtgagcagggcagcctcaagggagagccccttacatccttttctgggcacctctgtctaattaagttcaaacatacaagcctgattctaggattaaggcataagttagttctgaacagaaaaatataaggcagttcacatacacaatgaattcttgaagaaagaacctatagcccccagaccgataataaataattactgcctgccaaaatcctgaagaaagggaagcatgggggctccaaaattaatagtctcctgagaatacttatgagaccttgattttgtcacaaggttgcaatggtgaattatttattaagcacccgcagatcagagaatcttgcaacaaccttctcttacaacccacctgttcacttatgaatgatatcaagctcaggatataatccatctttccaaagattttagtatctttagtttgggctgcagaagaaattttggctcacagcattatgcttaaaccaaaaacagtgaaagagctctagtgtcctgcttaatgtagagtaactttttttttttatcagtccttcctagccctagagggaagataggagaggaggtaggaagatggaaaagtccttagatgaatgagtgaggagacttctataccatcaacttcagagtgtactgatccccggagcctggttcaggaggctagtttcagatgatcggaggaaatggaggaggtagcttttattcccatgcatgaagatggggggaggaggtgggcactagactaattttttaatctaaaaaaaatcaagcctattacaaatctcatttttcccttgtccttttgcctcccaataattcacataaaatcgtgttatcatagtaagctaagagagaggggcagtagctatttctaccttgactccgtggtgacttcatctttttcatgagtgtcattacagatacctggatcatgatgatcctgaaaaacaggtcacagggccaacagctgatgagtatttggagcatcttattcccccaagatgaccactccctgtcttctgctacacctatattcatcgggggggggaggtgggaaaatagcaggattcttggggactctgagggcatgaatggagaattatttgattgaaaccacagagctcttgtacttagtattaaatattaaaatagaaatgaaaagttcagcttttctgagctgtaccatagcggccaatggaggaagtcttacaaagaaaatctatatctgcacaagactgaagccactaaaccgagtatacacaacataaagatatcttcctatttcaagtatcccttagggggcatgggtgcttagctatcctatgaaagacagagaagataagctcaattttagagcccagagcttgctccggaacagcaagatccacatgaacttaaactctttcccactgagcctaggggtggaatgtaattcctctgatggttttgatttatttccaagctagacttctctatttgatcattggcaccttggcagctcacacatctccgtggtctcttctgtagctttcagaaaaaaaaaaaaaatatatatatatatatatatatacacacacatacatacatacatacatatacataaccctgtttgggtaaggcctattgacagaagccagatatctgggtggaagttagaagatgggcaaggaattcttatctcagagtttcaacactgcgacaatgtggagagaagtctcctgggaaaatgcagatgcccaataacttccaaaagaatcagggaagttggagtatttttgagatttacagtgtctttacttcagtaaaacaagccatagcaacattatgctctgcagagtcttctgttcacctttggatggaaaagagctgcttctcctagggagacaactaagaccaagaacaaaactcccatctcctaggccaattattgttttctcctttcctttcttttttattctctttgttacataatggtgaaatctacctggccattgccagtcaaacattctaattgctaaatatcatacagaaattcatgaaaaaagagtagaatcagcagatttcatttaatcccaagggtctttgcagttttcatcctctccctcaaagttatctcttctttgagacacatgatcacccctttcctcttctgggtggccatgattccaaaatgaacttcagtggttggtgcttcacaaaaggaaagacagctataatataaaatagaccaaaatacttatatttttagccactgcaaagtgggttttatcgttgagaacaaccttccaagctccttacttggaaacccttcctcacctcccagcctcctggcagctctcctggttccccttcatgtttttggctcccctccccgactttcctttcctatgaaaatgggatatatcccttaaactgtattcttatctaaactatcagactgttaaaagaaacccttgacagtaatttttcatggttttctcatcattttttaacttaggaagttggagaaagaatgacaagcaggcaaatgaggtcttgttcctgagtgcttcaactcaagatcaagtgctgggatagtgagtggctcttagtggagagggatgtgctctggaatattaagccacctctgagcatgtgtcctggtgctctggtcaagatgcctgttgtgtatgtggaggtattttgtggcttttcttgttctctctttttaaatttatttcaaggcacaagccctttgtgatggaggatgtctagcttgaacatgctggtaatgtgacactatcctcagctccccttggccctgggttcataggaggcaagtgtttctaaatttctcgcccagtgagttgttgcatgttatttttaaccattaatattgtgtgtctattggaatgagaaacagaaggttgaatgggaaatagattcagtgacaagcagacaaaaagcttggttctgagaaatgttcttcactgagttgaaaaaaaaataatctagaaacaaagaagagggaaattggggtagaatggtttggtggtgaagagacgatgacacccacatccagatgctctggtgtattccctcagagatttgtcattccctgtgagctctggcagaaggagaggctgtgctgccattgatggctcagaaatggtgacaggctggctttagaaagagacgtaggctttcttccagacagacattgataagcattgtagctcgattcttcaaaaccttgtctacaaacctttagggcagcccctctccaatgatttccattctccagtcaagaagattctgagtggaagagccttgtgtgcattgtgattacattgacttaaattgggaagccaaggggcactgagaactgaatatatcaggggaatggagttagctgaacctttttttttcaatattggaatgattttggatcacaaagttaggtcacctctgagagagaaaatctgttaaataaaattatatcttcccatctccagtgaaatcactaggttgttaatgtgattactgtttacttcagaaagaaaattgctagattcgattacaaataggcattttacagattgatttaatcctgagtcttacacatattaacatggaatttactgactcagacatgatcttctaagctataaaatattactggcgggtagtagatcatatgttctaagcaaaccaggaaagctaaaacgttaagtaatcctcccaaatattcagcgtctgtgtatttcactatagctgaaggggatttgggagttaggccgcaggggagaggagggacaaagtgccatagattaatgaaactcggagtaacttttttcttacctattaaaaataataattaacccaaggacaattctcaaaccagaaaaatcatgaaaagtgagctctgcacttgccattggttaaaatttagaaacacttagaataaatatccaggttttgggggaggagttgtttccttgcttgcattgttttgtttactggagctcttattttggggatatttggtctgtggcttcagagcatgttttggataaatgacttatccatgttgatatttattgagtggaaacccatgccaggctgttctaggtgctggaatataagaacgaaccacacaaagtccataccctcttgaagtttgtatgttgatgagaggaaatggacaaaaaagcaaatcaatagatctcatgtcagatagtgcaaagtgtcatgcagaaaaatgaggcagattaagggggatagatgataggatgggtgtggggttgctatttttgagtaagcaaagatgacatttaagtaaaggtttggaggaaatgagggagtgagacttggggctttctatgcaaaggcaaagagaaagcaattgcaaaggccctgctgtcaaaccaaatgcttggcaaacttgaggaagagccgggaggccaatgagggaggaggagagtatgagaggtgaggtcagagagggggcaataacgcaagtcaggtagggccttgtaggacactgtcaggatttggcttttaccaagagcaagatgagaagttgctagaaggttcggagcatagagtgatatgatctgacttcagttgaagaggaagctttgggactgctgtgttgagagtataccctgagagggcagacaatggaagcggctgaaagaccggttaggaagctcttgcaataatcctggtaagagatcatagttgcttgggccatgatgttagcaggggaggtgataagaagcagttggattccagaaatgttttaaagaatcgatatcacacagtttgctgatgaattagacattgaataagagaaagaaaaatcttaagaataaccttagaatgtttggcctgagctattgaaagtttggagttgccatcagtgtaagacaggaatcctgcaggtgaggctgactgggaaggaaagccttatagtttaaggtgtctacagatgttgcaagtagaaatgatgattaggcaattagatatatggacctggagttcagggaaaatgtatagcctgaagatacatatttaaaatttgtatgcgtttggttggtatttgaagccatgatactggaactagagaatgagtagcgacagaagaggattctctagactgaggcccagggtggaggattgttcaaagctccaggagatgagaagggaccagcaatgggaggagtggttaatgggtaaagcaagtactattattgagtaggataagactatctggaaacatggaggtcatcccattgatgacctggaagagagctgtttcagtggactggtgggggcaaaagcatgatggggtggattcaagaaagaggggaaggagaggaggtagagacaacaggaataggcaacgctttaggagtttggctgtaaagaggacatggaaatagcagatcatagagaatgtgacgcccagggagggacttctttttttaagatgggaaaaataacagcccattttatgttgacaagactaatccaggagaaaggaaaaattgatgatgcctagggagagaggacaattgctggagtaatgtctttgatgaagtgagagggcatgggattcaatacaaacatagagggactggcttcaggggactaaccaggtttgtttgtattaatagcagggagatagtgcatttgggcacagatgcaagtgggctggtaaatgggataatgggagtatgctttttctgattgatttttagtaaactatgaaaggagttcatcagttaagagtgagtataggggaagagtattggaggttttggaaggaagaaagcatgaatgagtcatatagccatccggcaggaagagtgagaacaaaaatgtcggagacaccaaaaatataggagacatgctagggggcataaaggaattatttgagtttttatgattatgagtttaaagaaatttgttttcttcagtcaccttcagctgcttaaacagggtggcaagttagattggaacagggttggggtataaccaggtgaatataatgaaagcaagagatgtggagtcaagggcaactgcagtgtgttaactataatgtttggtcatggaatctaagctgggtgtagaggaaagtgaggatttttagggaaggaggaacaatgaagcatattagaggcagaggattgagattcaagctgggatcactcagttagtgagtcagaagcaataacgtagaaattaggaggtgggttggagaggtggatgcttgaggttatggagggcttgcagttattggtggtttggggtctgttgttggggtggtgggctgagataagatagaggaaaaggttatagaaggagcaggggtcaaggaactgagaggccaggatatgggaattatatagatattgaagtcaacaaaatgttgacttccatatgataggaatgtaaattatgacaggaatgagggagagtgattcgagaacatttcaatcattgcatcaagtaaggggtgcagctagcctagtctgatgtgcatcaaagctggattttatggttgttgcatgttatttttttttaaaaaggagggaggatggtctggaatggcaatgaggggcaagaagggcatctccacctccaggccccagggcatgaggaaggtgcaagtggaaatgctcaccacctgggagtgattcagggtcctcaaagaggagccaggtttctgtaagagcaagaggaaaggaattgccagggttgtggctggtgatgaaccactgattcttaggcaggatttgggagttggtaaaggaagggaaatgagctgagatgggagatgtgcatgggtatctggggatgaaagtccaagtgataagagggactgggggtcttgggcttctcgtggtgattgccctaagcgaggctgtgggcatttggggttggccctaattgtctctatgtcagattgaggtggtgatgctgcaagtgcccggggcagggacttgatggcagcacatggaactctggtacctcttcctcagtcctgatatttgcttttttaacttctactaaaaaccccactgctaactgtgataggccacaacatgtttttatagatgtcaacaaatgttatcctggattgagaccacattttgactatttatcctggaaagtacacattttcacaggaaatcttgtcctttctaaggttcccaactaagctagtctcgttgtgggggggtcataattatttgttcttacttaccttactccttgttcatctttaaaaaaaaaatcaaataaaaaggattggaaagcaactttgcttctgcccaattctgctaaatgaaagttttctctgacagatcattttgattttgtattttcttttgtgacagaaacagtgggacaggagcatggaaatgaatgaaagtctttaaaagccttgagagagcgttttcaaggatcaggagagggcttagaaagaaaaggactctgagtggggctgcatgaaaagggggtaagtgaggactcttggggagggtctgaaaaaaacaagtagtttggcaaggagagcagaaaaataaaattccactgtgtaggcatttggagatttagcaagagcaacctagagtctgcccccagagacactgcacgttgtttctgacacagaggatgcactaattctatttcctgtgccacctgaaaaaatcataatttgagaagagaaactgttaggccatttctagtgaactatttttccaaaccctcacggaaccctgaacagggtgtgaatgagatcacaactactgctgctgctggttcagcctctctgtccactaataaaggcaaacccttcagcatggtcatatcctgccagccgttttcccccaaaagattaaattatgttcatcttggctcatctaacgccacatcttgagttcttaaggtatcacagccaactcagtcataccttactttatgcactgagtacgcattaaactctatttaggaaggaagagggaagcaaaggaagccagggatggttttgtcatgacctctctttgctttctgacctcccggaatccactctttggaagatgtgcagaaaacaatgtgacttccatttaccctgcctcagatctcccctccttcagactggctcccacccctggttaatggggtaacctgggtacgcctatttgttaaaggaatttgtgggaagtgaggggcaaaattgttggagcagagggaatagatttatgggagctttgttacccagtgactatcccagcattaatacttcagagtagccctgacgcttgtggaaacatttcaagggagctagaggcaatgttacagtggagatcagagaagggatggggcagtgagctcttgtccctaggggaaatctcttttcctccacgtgacaggagccagtgctccaacagcatcagaattacacttagccacaatgactgatgcgtgtgtgtgcctgtgcacatgtgtgtgagcgtgtgtgtgagcgcacgtgtgtaagggggtgatgatggaaacatgatatgcatccactatggggttgcgttgcaaattcacagctgcacccctcccaaagtcacatggcagaggggaaatggtcatagctttcctgtgtgatgtcctctccagggagcaccgacgactcaggcttgcctcagctctgcgaggcccagactccacactcctccctggggccggctctccttcctcttgactctcagatccccagcccatccacctccaagttctttttttgtttatttgttttgcttctgttttctgtaaatatctatttcttattttggacatgcagggatcaattgagattctggagtggggggaggggcggatgactgggtctttttattttttcctttctctcttggattttggtgtttgaacttgaaaaaaataattacaagtatatatataagcaaatgacttacctttaaacaaaagaaataagttgatttcattcttttgatttatcttgtttctcattggggtcgggtgggggtggggggtcttttgggtggagggcttttttgtgagctgtatagatttccagtttggacttgttcaaatgatgcaggaacaaaaattaaaacgaattaaaaaaaagacaacataaaaaaacaagaaaaactttgtgatgtataaaagctgtaaatagtcaaagattctttctcttttctaatggcaaattatttctgtcactttatattcaaaaaataaagaaacctaccacaaattatcagggttaaaaaaaatccagaatcagagtttttttcttggtctttgtgctttctttactgtgattccaaaaagattgttaaaagtgctgtttgggaaaaaccccttgcccagttggtttccaattgttggtaggagtcttggactggcgaatctacttgggttgggatctgtttctactatagctggtgctggagcccttggttacactcatctccccctcatgtattattctgaaacaaatcccataattatatggtttcatctgtaaacattacagtatatatctcttttactaaggactcttttttagtttaacaataccattgtgacatcataaggtagccataattccttgataagaaatatccagtgttcactaattgtctcaaatgtaatactgttttatggggtggtggtgggtttttttcccataagtcataaggtccatacaattagttgctatttctcaaatgtattttaaatctaaagatttgctttctaactttttcttgacatatacttgttaaaggaatagagcactttatcctttagaggttcccacagttggattttgctggttcttcccaatattaatgaagacaatgtaaattggtaattgttttactctttaatgcatacttccattgcttggaggtgtcagaaaagcaaaccagcccttgttttgctgattgtttcccaccccactcccagtcccagtcctgttactggctttatatatggattctaaatgatctctctttctataccttgattgctaaattatccaagttaaataatagtatctcccccttagctatcttggtaagaaaatagcatagacatatttagcttttcacatgccaatcatgaggcacatttcaacatcactttattaaaagccacatgctcctcctatagtcctagtactggaaaggaccttctagagaccatttagaagaactctgtctcatggccaaggagcctgggtgagctcatgtatggcagccctggctagaagccagcagggctaatccctagttccaggaatctcccaggataatgtattgttatctctttgaagttactgtctcagagagattatctgtgagggtctctcttgtactttcctttgtactgaacaaaaattcttgtttctccctgaacattccagaaagtagcaagctctaacataaagccagattcttcccagatatagtgattctctttacttctggagatacttccttaaaccccaaattttttttggacaacagaactgctttaacaataatctgaaaattatacagtaagtttgatttgaaaatctgagatttgtttaaaaaaaaaagggggaagacaagacttgaaatcttggctctgatttttctgttgtttttgtttttttgtttgttttttttttgtttgtttgttttttaagagggtcttgctctgtcatccaggctggagtggagaggtatgattacggctcactgcagccttgacctcccaggctcaagtgattctaccacctccgcctcctgtgtagctgggtactactggtgcataccaccacactgggctgattttgtattttttgtagagacggggtttcaccatgttgtccaggctgattctgaactcctgggttcaagtgatctgcctgcctcggcttcctatgatgctaggattacaggtgtgagccactgtgcccatcttggctctagttttaaataaggaagcttgcgagtatttatctggattatggtgggaacctccagtaggatatcatgaaagctttgatcatgatcacaacaccttcaataaaacaaagactccaatgacaagggtgagtgccagaacttcctgagactatgccaagtgtctgttctttatacagcctgcctcacccacacctggaatctcttaaatggattagctcagagcacctgtgtcctacatgcccttgtgccaccctgaagtccactgcaagggactatcagtaagaccagtctaatgaaaggctgagggagcacccggttgtgtccccatcagccaactgttccggaaagatgagcctccttggctgtcccaggattgacactcttcccctgcatacatcactaggaaggtagaacctaccctgaacacttgatttgaatatttctattcccctattcttttacaaatgcaaactaattctttcaaaaatataaatgatcctctgaaagaaatttcttgtggaattctggaatgaccaagaagatgggaagagaccaccagaaaagtggttctgagagtctgcagggcctgtcaaaaaaattatgccaaagctatcatttgtggcctgcatcaaatcatctagacttccgttttcttatcttgaaacccagatgttggtctatctgccctccccatcaaaggttacctggagcagtagttctcagttttacatagtggagtcatctgagtatttctgaaatgctgatgcctgagtctcatcccctagagagtctcattcaattgtcctgggtggctggaggggttaggggttgaaaacttgcccaggtgattttaatatacagccagaatgtgaaccactatgtatgtgcatctggggaaagcgggcctcctatttacttctaatcccattatatgtggcagctgtttgttgtcactctcctctcaccatggacaactattcacagcacagacagtcaccctttggaactccaggacctctttcctcttccatttggagctgaagaatggacataacggaagcaccattttatgattacagcctcgttctctaattctgcctatccccagcatacacggctagatatagacatactgcaaagctcctctatgtaaaggaaattctacacataaaatttaaacttggaatcga

SEQ ID NO: 20 - DNA, 14706 bp, Homo sapiens
ctctttctctccctacctccctcgctcagcagctcccggtcgcacaactcccagcagccggcgctggggaggtggtagggggtgcgggtggaggtgcccagttagcttctcgtgggtggccaacctggggctcccattctcctgtgcaccctggagtggagctcccgctatcagccacaggggctgaggatccgcactgggggcgtcgtgggcatcgggcttcagcttcccccctcttgtctagcccccctgggcgcataggggggcttgtgagcactgcagactgcagccttcccctcaaccctccctcccctcgttggtgggctgaccctgcctctccttctctcctccgccccgttagagccctgctgcagagcctccggctgggatagccgccccccgtgggggcgatgcggacagcgcgggacagccaggggagcgcgctggggccgcagcatgcgggaacccgctaaacccggtggctgctgaggcggccgagatgctcgtgcgcgcagcgcgccccactgcatcctcgaccttctcgggctacagggaccgtcagtggcgactatgggcagagtgggctattggaccctgctggtgctgccggcccttctggtctggcgcggtccggcgccgagcgcggcggcggagaagggtccccccgcgctaaatattgcggtgatgctgggtcacagccacgacgtgacagagcgcgaacttcgaacactgtggggccccgagcaggcggcggggctgcccctggacgtgaacgtggtagctctgctgatgaaccgcaccgaccccaagagcctcatcacgcacgtgtgcgacctcatgtccggggcacgcatccacggcctcgtgtttggggacgacacggaccaggaggccgtagcccagatgctggattttatctcctcccacaccttcgtccccatcttgggcattcatgggggcgcatctatgatcatggctgacaaggatccgacgtctaccttcttccagtttggagcgtccatccagcagcaagccacggtcatgctgaagatcatgcaggattatgactggcatgtcttctccctggtgaccactatcttccctggctacagggaattcatcagcttcgtcaagaccacagtggacaacagctttgtgggctgggacatgcagaatgtgatcacactggacacttcctttgaggatgcaaagacacaagtccagctgaagaagatccactcttctgtcatcttgctctactgttccaaagacgaggctgttctcattctgagtgaggcccgctcccttggcctcaccgggtatgatttcttctggattgtccccagcttggtctctgggaacacggagctcatcccaaaagagtttccatcgggactcatttctgtctcctacgatgactgggactacagcctggaggcgagagtgagggacggcattggcatcctaaccaccgctgcatcttctatgctggagaagttctcctacatccccgaggccaaggccagctgctacgggcagatggagaggccagaggtcccgatgcacaccttgcacccatttatggtcaatgttacatgggatggcaaagacttatccttcactgaggaaggctaccaggtgcaccccaggctggtggtgattgtgctgaacaaagaccgggaatgggaaaaggtgggcaagtgggagaaccatacgctgagcctgaggcacgccgtgtggcccaggtacaagtccttctccgactgtgagccggatgacaaccatctcagcatcgtcaccctggaggaggccccattcgtcatcgtggaagacatagaccccctgaccgagacgtgtgtgaggaacaccgtgccatgtcggaagttcgtcaaaatcaacaattcaaccaatgaggggatgaatgtgaagaaatgctgcaaggggttctgcattgatattctgaagaagctttccagaactgtgaagtttacttacgacctctatctggtgaccaatgggaagcatggcaagaaagttaacaatgtgtggaatggaatgatcggtgaagtggtctatcaacgggcagtcatggcagttggctcgctcaccatcaatgaggaacgttctgaagtggtggacttctctgtgccctttgtggaaacgggaatcagtgtcatggtttcaagaagtaatggcaccgtctcaccttctgcttttctagaaccattcagcgcctctgtctgggtgatgatgtttgtgatgctgctcattgtttctgccatagctgtttttgtctttgaatacttcagccctgttggatacaacagaaacttagccaaagggaaagcaccccatgggccttcttttacaattggaaaagctatatggcttctttggggcctggtgttcaataactccgtgcctgtccagaatcctaaagggaccaccagcaagatcatggtatctgtatgggccttcttcgctgtcatattcctggctagctacacagccaatctggctgccttcatgatccaagaggaatttgtggaccaagtgaccggcctcagtgacaaaaagtttcagagacctcatgactattccccaccttttcgatttgggacagtgcctaatggaagcacggagagaaacattcggaataactatccctacatgcatcagtacatgaccaaatttaatcagaaaggagtagaggacgccttggtcagcctgaaaacggggaagctggacgctttcatctacgatgccgcagtcttgaattacaaggctgggagggatgaaggctgcaagctggtgaccatcgggagtgggtacatctttgccaccaccggttatggaattgcccttcagaaaggctctccttggaagaggcagatcgacctggccttgcttcagtttgtgggtgatggtgagatggaggagctggagaccctgtggctcactgggatctgccacaacgagaagaacgaggtgatgagcagccagctggacattgacaacatggcgggcgtattctacatgctggctgccgccatggcccttagcctcatcaccttcatctgggagcacctcttctactggaagctgcgcttctgtttcacgggcgtgtgctccgaccggcctgggttgctcttctccatcagcaggggcatctacagctgcattcatggagtgcacattgaagaaaagaagaagtctccagacttcaatctgacgggatcccagagcaacatgttaaaactcctccggtcagccaaaaacatttccagcatgtccaacatgaactcctcaagaatggactcacccaaaagagctgctgacttcatccaaagaggttccctcatcatggacatggtttcagataaggggaatttgatgtactcagacaacaggtcctttcaggggaaagagagcatttttggagacaacatgaacgaactccaaacatttgtggccaaccggcagaaggataacctcaataactatgtattccagggacaacatcctcttactctcaatgagtccaaccctaacacggtggaggtggccgtgagcacagaatccaaagcgaactctagaccccggcagctgtggaagaaatccgtggattccatacgccaggattcactatcccagaatccagtctcccagagggatgaggcaacagcagagaataggacccactccctaaagagccctaggtatcttccagaagagatggcccactctgacatttcagaaacgtcaaatcgggccacgtgccacagggaacctgacaacagtaagaaccacaaaaccaaggacaactttaaaaggtcagtggcctccaaataccccaaggactgtagtgaggtcgagcgcacctacctgaaaaccaaatcaagctcccctagagacaagatctacactatagatggtgagaaggagcctggtttccacttagatccaccccagtttgttgaaaatgtgaccctgcccgagaacgtggacttcccggacccctaccaggatcccagtgaaaacttccgcaagggggactccacgctgccaatgaaccggaaccccttgcataatgaagaggggctttccaacaacgaccagtataaactctactccaagcacttcaccttgaaagacaagggttccccgcacagtgagaccagcgagcgataccggcagaactccacgcactgcagaagctgcctttccaacatgcccacctattcaggccacttcaccatgaggtcccccttcaagtgcgatgcctgcctgcggatggggaacctctatgacatcgatgaagaccagatgcttcaggagacaggtaacccagccaccggggagcaggtctaccagcaggactgggcacagaacaatgcccttcaattacaaaagaacaagctaaggattagccgtcagcattcctacgataacattgtcgacaaacctagggagctagaccttagcaggccctcccggagcataagcctcaaggacagggaacggcttctggagggaaatttttacggcagcctgtttagtgtcccctcaagcaaactctcggggaaaaaaagctcccttttcccccaaggtctggaggacagcaagaggagcaagtctctcttgccagaccacacctccgataaccctttcctccactcccacagggatgaccaacgcttggttattgggagatgcccctcggacccttacaaacactcgttgccatcccaggcggtgaatgacagctatcttcggtcgtccttgaggtcaacggcatcgtactgttccagggacagtcggggccacaatgatgtgtatatttcggagcatgttatgccttatgctgcaaataagaataatatgtactctacccccagggttttaaattcctgcagcaatagacgcgtgtacaagaaaatgcctagtatcgaatctgatgtttaaaaatcttccattaatgttttatctatagggaaatatacgtaatggccaatgttctggagggtaaatgttggatgtccaatagtgccctgctaagaggaagaagatgtagggaggtattttgttgttgttgttgttggctcttttgcacacggcttcatgccataatcttccactcaaggaatcttgtgaggtgtgtgctgagcatggcagacaccagataggtgagtccttaaccaaaaataactaactacataagggcaagtctccgggacatgcctactgggtatgttggcaataatgatgcattggatgccaatggtgatgttatgatttcctatattccaaattccattaaggtcagcccaccatgtaattttctcatcagaaatgcctaatggtttctctaatacagaataagcaatatggtgtgcatgtaaacctgacacagacaaaataaaaacagttaagaatgcatctgcactgtagtgcgattgacatgtgcaagagattaggaagtttggctcgtaacagtttcagctttcttgttatgccttccatcacagcccaggctcaccccaagaactccaggctcccctaaagaatagcaaatcagtgtgttcgtgatgactgtgctaccttcattatagttcatttccaagacacatctggagccaaaggcccgagggaccctcaggtggggagagctacaggaatctctttggatgttgatgtgtgtttctctcaccctcggcttcgatggtcttgttcagagctgcataaactaacacatttatgtctccgagatctaagtgtggatcttctgtctgtgacacagtggccattgtagtttatcccgaagacgcctatgtacgtaagtttgcatttcctcccttctggtgatgactcagggttgtatagtatctgttaccccttccctcccagagtaaccataactcgttccgtttccaaacagccatggtggtgtccaattagctgtgtatcgctcttcccagagttgttaatgtggtgacatgcaccaacagccgtatgtgtactgtgatctgtaagaagtacaatgccatctgtctgccgaaggctagcatggttttaggtttatcttccttcacatccagaaattctgttggacactcacttccaccccaaactcctcaaatcaaaagccttcaaaacacgaggcactcttggatctaccctgagtatcctccaaactgtggatacagtttagtgagacaagcaatttctcccttctgagttattctctctgttggtggcaaaccacttcatagcaccaacagagatgtaggaaaaattcctcaaagtatttgtcatttctgagtcgcctgcattatcccattcttattctcctcaaacctgtgcatatatgacatgaaatgatatccattttttttttaagttagagacagagaggggaatacttatgcatggggagcctgttagcacagtgcctgccacaaaaacaagtgcccccgacaagatagttgctatgttatgacactttctcagatcaggattttctagtttaaaaattaaatatcataaaaccagcaaataatattaactaaaccttttatatagttctactctgttaattagtgtatttcatgtacatgaatatttttgaagtatttgggataactagccatcagtgtttgcagaagacctcagtctgttttgggccattacggaaacactaagctaatgtagactcgcctggccatttcctggtttcctcctgcattctgctttacattcttgaagaaagggataaagtggcaggttgaaaaattatcccatttcttctcctcatggtgtgcgttctgttttgtaagtattattttcttgcatgtgggcaagtgtcagggatgattcccttaaagctcaaggatgcatggaatagctccatctttgggaaagtacacctgacctacagtgttcctataatgaaaagtggttgcaataaatttcagggtcaatcatagggagacagagggaggcagtgggtgagaggagcttcctttcatttttgaaaggcagaaggggctgactaggtgctggggatgcatgtgcttgaaaatgacattttatttcccggatgctgtacctctgctgccccaagacccagccatggagaagcctggatcgaagggcccagaatagtcaccccaacatctgatcctaaatctgaatctccatgactgtgtagccttttagccctttctaccttctgcccttccagtgcattccagaccttaatccaccccagcaaggatcgcctccatttcctcatgagtctgggatgttttgacatgaatgaattaaaaagtcttcccagaatacagaagagtgatttgtcgtcaagccaaagaggctttctgtcttcctctggtgtcctctaaacccagggggctgccaaatgccacagagacctccagggagagtgagagttatacttggactgacttcatcggtcagaaagccttcccctaacagcagcattatttctgctattaaccccatacatctctggataaagaaactgaaaacgtggcctttggaagaatcaacagtaatgttgtgaaaaaaatatatattcttttcactgaccgaagtgttttttcaaataaataatcagaaatgttatgctacacaaagtttatggtatcccttctctactaattcaatgactcttcaatgtgatgatcccaagcaaaaaaaaaaaaaaaaaaaaaaaatcaactactgatcagatggtcaatttctaaggaaaagatgtagagttattcaaaggtcatctaagtcagttcagtctttgcaaaaagaatatcaggaatgtaacgtccagtggtaaatggaacccacagtatgagaagtaacaagaattaaataggaagcctggaagcctgagactatagatctatcacagaagattcccgtaacatatcccagctctgagttcttagatttgttgatgtcaaggagcccaaatttctgaacacaaatgcctgagtttcagctggcttaagtcagagctggcatccataatgatattttgttaattctaattggttttccccacttgagaaggacccaactagtcctcagataggtacacttgaatgcaaacggttgggtctctgtccatgggtaatagcacgcacatgcaaccaatttaaaatactctgggtaagagagtcttctagagcataaactgtacttgagaaacctttgtgcttctggaaaatgggtttcatcatccttacatctgcaggttcttaggccacttatggtttcaatgctctgagctggcaagtccctataggtctatctttctgccagagagacttgcaggattataatctcacaatctcaccactcattactgttatgtcctctgcaaatctctctgcgttattttagccagcaccatgactgggcatccacggattactcttgattttctcttccctgattcacaggatgccagagagtctgagtatttagtttttctggtgaaagcattaaaattcacacattgcagcaaaatctgtcaaatcatttcacgcttattttctggggagaaaaatagaaaattacaccttgtttacaaatgaaatagtttcacttaaatgggactctagccaaggcagttaattagtcaaaagatccacagctgtcatatagtcacacattgtttattctatttgctatgtcgaccactgatgtgtatttaatattctgcatccttcacaggaaatgacaaatgccctgaattatttgcaaatagaattgctaatgaaaaagaaaaaaaatgttgatgttaattaaatggtgattaaaaatttatctcctttaaatctaaaaagtgcagctttaataatcagttgtcatataaataagtaccttgtactgaaatcccttgcggtttaagacctaaataaacactaatttgagaactctaaataccttacagaggagctggatatattcgttaacattatgcacattcctgtactgtttctttgtgcatagctgttgtgcatgaatccttgaaaaggggacatgcgttgtttctgaaatggagcctgtttgtattaaccatggctatagtagacatgattggactcaaagaaaaagaagtttgagtaggtggaagctaaatgtgttgcttgatatttaatgagtgaagggttctatgtaaaggcaattttccttccaaaagtactgtttattttcactggagacaaaattagacctttccagataccccatctctttttggtgaacagggttgaaagtgctgtggggtctatacttgaaaagttctcccaatatgcagttactgtttcccattcaatccctatacattgtactgtgttcatatctaattgtatcagatgaggtcagcttgccacttgatatagatcagagatgatcaaatgcacttatcctgtgaatacaactgcttgtgtacacatgtatataatatacatgcttgatttctacaattcacacgtaagtgtattatccaaataaaattgtacatattgtaaagtttatgttaaaaattaaatggtgatgatggtattaacacatggaaaacccattctggttatttttatgggaggatatttctgtagagagggcactatatttttttaatctcagttgtagggaggatgacagagcctaagtagaatatgggtgttggcgttcttggccccttctttaaaaaacaaaactctgggatgaggggtatagaatatattctgctcgcatttgcttctgaagacaatgccgatatggacttgtgtgtgatttccttaaacacatcaaagggcatagatttcctatttaaattccttatcctctttcctcacctgcactctttctgtgcccagcagtactttcagtatagtattagctagaagtgcctgcccagaagggtcttagaaaaatagagttcgtagaaccacaccaaaagttaggagaaggggaggagagatgagaaaaatgaagtttacaaatgggcagttgcctgtttaagatcatcaagttagagagtatcagagcgaggattagatccaagacatggttcaatttctggctcgattctctgaccgggctgacttatttctaggtttctttcaaggggaaactcctgagtagctctggaataagagccagagatccccataaggctgccctggtttttgttttgttttgttttttgtttttgccacttgccccagaaagacctacccatgcttctttgaatttggttttcctttaaactccctcacaatccctgtcttaaagttcctagaagagaaaacttttctcagagaccgtctccttaggaaaaatcaacagggatattgtttaaaacctcaaactgaagataaaaggctggcagtgaccattgaaataattgaggtctatgtatcatgcattgaggaagaaccaagcacattgaccagctaataggacttcactgaatgtccactttattttgaaagccagtttgggattgtgggggaggggatgcaaaataaatagaaaatatagtctcttcccataagtagcttatggtttagaagttcagttctctaatacaaaggataaccaaggacccatgcaatgcaaacagagactaaacagcaaaaattagaaatgacaactaaagttgtagccaggaaaaaaaaaataatctgattctatgagcataccttttttaaaaaaaaaaaaaaagtccttccctaaacctttaagtaactccggagaatagagttagtggtcccggaaatgcagttgatttattttaatcttcaaataatggacttaggagacccaggagctgctaaccaggtgaacacactcacactcctacatttttgcacaggaaggcttttgtctggttccaaccagttctccagtccttgccaaatcatgcctcctcccagccttgcattatgacatgtgcctcaagaaaaggttgaaaaaattccccaaaggattagaaaatagaccatgatgtaaaaagacgaagcatttccctaaagcttttttgtttggttttgtttttgacatttgaagaacccttctgatacaccagggtatgaatgtatcagggaagcatagagggaaaattctttatcaagttccaagtgcctactgctgtggctgacagaaagcatcaggttccctagagagaaaacggggcaaagagatggcaaaaggaatagaggtgtatttcaggaaggatggcgaggtggagagggagcttttgtcactctgtctatcccattctgtgcattcttggaatgttacattttctgcttatcgcaagtacttgccatggatctgcacctgaatttgaaaatatcaaaatatatctaaattgagtgtaggcgatattctaggcatatcctagaatgtgatcctcaaaacaaccttaagatataaatatgattgctcccattttacagataaggaaactgaggctcaggtttttctcaacctcgtcagcaactagaattagctagtaaatccctaacgttagacatattttcaaagtgcctggtctcctccagggaggtcagaatagaagtcagaaagagaggtgtgctcttttgaggttcctgggcatatacccttgagtatcagcctctgtgacagggaagtctgttagggtcttacccctcattgcctgatctgtgtgtatggagaacttctggggatctcacctctgttcatagttccttctgagacttgaacactgtttgtgtctccctttcatatgcttgcacttagcatttaaaaaaatttctggggagttattttctctacttagcatgtgcctaatcttaaatatctctttcctaatgtctcatctttttatcagtcaacatcagggctaattccattaaacatatatacatagtacatgctctcatgtgtgtgggtatgcacacacacacacacagccatgtgaccctccattaaagtcctcctaaatattgtgacccatgttcaaagaggtgatattatttgcaagttgactattcctcctatttcatgtctatgtttggttatctttgccaggcagatggaatgagtttcagaggtgggacagggaataggaaatagagtttctgcatgaatgatggagagctttggtagtcagaatgattgaggaccagctattaaaatgttgttgttgttttccttcttggtctgttcctttttttttttcttttccaaattgtcttagagataattcccggctcacttagcattaggataatgacggttattctgtcccactgaagagtgttgaactggaacgcttccaccgggctttataggtacccagaatggcctaatgtttaacatatcctgcaggcatgcccctgagcacacactgcctttcctgcattcccccctctccatggtctaagccacatttctttcctggcatttatatttactgagaggtgaggggtgcattaggcccatttgatggatgaggacactagcggggatttgaaggaatgtctttctgggagtctccatgaattctctggagagttagctaacttaatgcaagttaagtgctagtctatgagaaaaatttctcagctgttttcatctttcaagctggcagtaaaaagccttttaagacagaaagattattatttgttatttggagatcctgttgttgggtctacaagtgcgtgaccatgttctccttggaatgttgagctatgcagaaatggagcactccaaagaacgatttgaccaatagcatttcttctctgggggttgtatttcaaagcatgcaactctccagggaaccagaactaaattgcttaaaatgaagtcattcctcagattaacttcctcagataaagtgtcagcggtctgcagaaacgaagaagacaaaactgagattatcactcataattctcttacttactatgtcagtgaaacaatgagtttgcatttttgcaatcctagaacattcttcattagccctgggtcatgacctcttccagttaattctctttcacacctttaggaaagatttaagatgaaccttcaataggatattaacataactcatagccaataccacagctgcctttcaaattaatgaggttaattgttctccagcaaacatgagtttgtctttggcattttaaatgcttcccattgatctgacattttgctgtttcaagttttaaagggctcaaatcaaagactattgataactgagcaaagagcgaagatccagaaatacgaaaacattgtctttttttttccatgaaaaacaatcatagccttttgaattcaatcgaagtttctacattagccatctaagacttatttaattatttctgttctcagtcaagctaattcaagtgaatgaacagtattgacttttaaaatcttttttaaatttttttaaatctttagtttattaagtttgtagaaaagctctggggccatgaccacttacgtaaatgtttcagtttaaaaacaaaagattcaggcctctaatttgagccaaatccaggtgatcttgtttgaaatttttgatgaatttgaaaagatgaaagtggaacttttaacattcatgttccccaaatttttcactgggaagggatgctaattgcctacttaagatataagttcaagaataacattttcatagaaaattcagaaaactgcttgacacagcagtgacatagttagatgtggctcagatgccttccaaacctgagggtccccaaagatttctttaccagttgtttttaactatgaatcttaatcttgttcattcccctgccaaaacaaatttaaaagcataaacctgctgaattaattggcagaattcaccatagtttatatcaccccagaatgtttatgtgtatatgtttatgtatagtatatatatttcaagctgaatgttcagaaagaataaggtgagtttcttggtagcttgaataagaattagatcataaaaattataaataatggccttctcaaagaggataaatgattcatttttcactcacgtattaataatatgacttgttgcataatacacaagctggtcattgcctgcaagaaagttattgaattaagcagctatccaggtttatgtctgagtccatttttaacccaacaaggctaggattgaactccatatgcagagggtttctattcacagctacattcaaacagaatccaaaacccacatcagaactgacttgatgtcttgtatggtggagctgaaagcaagctgtctgctctaagcaatggtcttcatttgggaattaatgttcactgatcaactgcagcagtttatatctgtcatgttttaatccactgcatgaggaagtgctaataagtgtattaaatatttccaagcatagctacaaaattatatgtttgcttaacacctttctatgatagaggaagtatatactatggttgtcttcttgtagtatgtcttttaattttgttcaatgtgttatcaactgttttaaggaatggggattgaaattcgtgtgcaaactgatatgtatataactcctgaggcaacagaagtgcccggtcatatggtgtaaaaatgtaaatacaggaaaatttcatttttctaataaagataatttctgccaattgatatgaaaatga

SEQ ID NO: 21 - DNA, 14600 bp, Homo sapiens
actcacacacacactcgccctcaaacacaccagcccggagctgggttgcggagagggtgctccggcgcgctcagaggaccgggcagttgccgtccggagtggggcaggaagcggagctagggatcttggaggggagctcctagggcttggccggatcatgggaccgggtgagcgctgagaatcgcggccgcagccatcagccctggagatgaccaggagcggccactgctgagaactatgtggagagaggctgcgagccctgctgcagagcctccggctgggatagccgccccccgtgggggcgatgcggacagcgcgggacagccaggggagcgcgctggggccgcagcatgcgggaacccgctaaacccggtggctgctgaggcggccgagatgctcgtgcgcgcagcgcgccccactgcatcctcgaccttctcgggctacagggaccgtcagtggcgactatgggcagagtgggctattggaccctgctggtgctgccggcccttctggtctggcgcggtccggcgccgagcgcggcggcggagaagggtccccccgcgctaaatattgcggtgatgctgggtcacagccacgacgtgacagagcgcgaacttcgaacactgtggggccccgagcaggcggcggggctgcccctggacgtgaacgtggtagctctgctgatgaaccgcaccgaccccaagagcctcatcacgcacgtgtgcgacctcatgtccggggcacgcatccacggcctcgtgtttggggacgacacggaccaggaggccgtagcccagatgctggattttatctcctcccacaccttcgtccccatcttgggcattcatgggggcgcatctatgatcatggctgacaaggatccgacgtctaccttcttccagtttggagcgtccatccagcagcaagccacggtcatgctgaagatcatgcaggattatgactggcatgtcttctccctggtgaccactatcttccctggctacagggaattcatcagcttcgtcaagaccacagtggacaacagctttgtgggctgggacatgcagaatgtgatcacactggacacttcctttgaggatgcaaagacacaagtccagctgaagaagatccactcttctgtcatcttgctctactgttccaaagacgaggctgttctcattctgagtgaggcccgctcccttggcctcaccgggtatgatttcttctggattgtccccagcttggtctctgggaacacggagctcatcccaaaagagtttccatcgggactcatttctgtctcctacgatgactgggactacagcctggaggcgagagtgagggacggcattggcatcctaaccaccgctgcatcttctatgctggagaagttctcctacatccccgaggccaaggccagctgctacgggcagatggagaggccagaggtcccgatgcacaccttgcacccatttatggtcaatgttacatgggatggcaaagacttatccttcactgaggaaggctaccaggtgcaccccaggctggtggtgattgtgctgaacaaagaccgggaatgggaaaaggtgggcaagtgggagaaccatacgctgagcctgaggcacgccgtgtggcccaggtacaagtccttctccgactgtgagccggatgacaaccatctcagcatcgtcaccctggaggaggccccattcgtcatcgtggaagacatagaccccctgaccgagacgtgtgtgaggaacaccgtgccatgtcggaagttcgtcaaaatcaacaattcaaccaatgaggggatgaatgtgaagaaatgctgcaaggggttctgcattgatattctgaagaagctttccagaactgtgaagtttacttacgacctctatctggtgaccaatgggaagcatggcaagaaagttaacaatgtgtggaatggaatgatcggtgaagtggtctatcaacgggcagtcatggcagttggctcgctcaccatcaatgaggaacgttctgaagtggtggacttctctgtgccctttgtggaaacgggaatcagtgtcatggtttcaagaagtaatggcaccgtctcaccttctgcttttctagaaccattcagcgcctctgtctgggtgatgatgtttgtgatgctgctcattgtttctgccatagctgtttttgtctttgaatacttcagccctgttggatacaacagaaacttagccaaagggaaagcaccccatgggccttcttttacaattggaaaagctatatggcttctttggggcctggtgttcaataactccgtgcctgtccagaatcctaaagggaccaccagcaagatcatggtatctgtatgggccttcttcgctgtcatattcctggctagctacacagccaatctggctgccttcatgatccaagaggaatttgtggaccaagtgaccggcctcagtgacaaaaagtttcagagacctcatgactattccccaccttttcgatttgggacagtgcctaatggaagcacggagagaaacattcggaataactatccctacatgcatcagtacatgaccaaatttaatcagaaaggagtagaggacgccttggtcagcctgaaaacggggaagctggacgctttcatctacgatgccgcagtcttgaattacaaggctgggagggatgaaggctgcaagctggtgaccatcgggagtgggtacatctttgccaccaccggttatggaattgcccttcagaaaggctctccttggaagaggcagatcgacctggccttgcttcagtttgtgggtgatggtgagatggaggagctggagaccctgtggctcactgggatctgccacaacgagaagaacgaggtgatgagcagccagctggacattgacaacatggcgggcgtattctacatgctggctgccgccatggcccttagcctcatcaccttcatctgggagcacctcttctactggaagctgcgcttctgtttcacgggcgtgtgctccgaccggcctgggttgctcttctccatcagcaggggcatctacagctgcattcatggagtgcacattgaagaaaagaagaagtctccagacttcaatctgacgggatcccagagcaacatgttaaaactcctccggtcagccaaaaacatttccagcatgtccaacatgaactcctcaagaatggactcacccaaaagagctgctgacttcatccaaagaggttccctcatcatggacatggtttcagataaggggaatttgatgtactcagacaacaggtcctttcaggggaaagagagcatttttggagacaacatgaacgaactccaaacatttgtggccaaccggcagaaggataacctcaataactatgtattccagggacaacatcctcttactctcaatgagtccaaccctaacacggtggaggtggccgtgagcacagaatccaaagcgaactctagaccccggcagctgtggaagaaatccgtggattccatacgccaggattcactatcccagaatccagtctcccagagggatgaggcaacagcagagaataggacccactccctaaagagccctaggtatcttccagaagagatggcccactctgacatttcagaaacgtcaaatcgggccacgtgccacagggaacctgacaacagtaagaaccacaaaaccaaggacaactttaaaaggtcagtggcctccaaataccccaaggactgtagtgaggtcgagcgcacctacctgaaaaccaaatcaagctcccctagagacaagatctacactatagatggtgagaaggagcctggtttccacttagatccaccccagtttgttgaaaatgtgaccctgcccgagaacgtggacttcccggacccctaccaggatcccagtgaaaacttccgcaagggggactccacgctgccaatgaaccggaaccccttgcataatgaagaggggctttccaacaacgaccagtataaactctactccaagcacttcaccttgaaagacaagggttccccgcacagtgagaccagcgagcgataccggcagaactccacgcactgcagaagctgcctttccaacatgcccacctattcaggccacttcaccatgaggtcccccttcaagtgcgatgcctgcctgcggatggggaacctctatgacatcgatgaagaccagatgcttcaggagacaggtaacccagccaccggggagcaggtctaccagcaggactgggcacagaacaatgcccttcaattacaaaagaacaagctaaggattagccgtcagcattcctacgataacattgtcgacaaacctagggagctagaccttagcaggccctcccggagcataagcctcaaggacagggaacggcttctggagggaaatttttacggcagcctgtttagtgtcccctcaagcaaactctcggggaaaaaaagctcccttttcccccaaggtctggaggacagcaagaggagcaagtctctcttgccagaccacacctccgataaccctttcctccactcccacagggatgaccaacgcttggttattgggagatgcccctcggacccttacaaacactcgttgccatcccaggcggtgaatgacagctatcttcggtcgtccttgaggtcaacggcatcgtactgttccagggacagtcggggccacaatgatgtgtatatttcggagcatgttatgccttatgctgcaaataagaataatatgtactctacccccagggttttaaattcctgcagcaatagacgcgtgtacaagaaaatgcctagtatcgaatctgatgtttaaaaatcttccattaatgttttatctatagggaaatatacgtaatggccaatgttctggagggtaaatgttggatgtccaatagtgccctgctaagaggaagaagatgtagggaggtattttgttgttgttgttgttggctcttttgcacacggcttcatgccataatcttccactcaaggaatcttgtgaggtgtgtgctgagcatggcagacaccagataggtgagtccttaaccaaaaataactaactacataagggcaagtctccgggacatgcctactgggtatgttggcaataatgatgcattggatgccaatggtgatgttatgatttcctatattccaaattccattaaggtcagcccaccatgtaattttctcatcagaaatgcctaatggtttctctaatacagaataagcaatatggtgtgcatgtaaacctgacacagacaaaataaaaacagttaagaatgcatctgcactgtagtgcgattgacatgtgcaagagattaggaagtttggctcgtaacagtttcagctttcttgttatgccttccatcacagcccaggctcaccccaagaactccaggctcccctaaagaatagcaaatcagtgtgttcgtgatgactgtgctaccttcattatagttcatttccaagacacatctggagccaaaggcccgagggaccctcaggtggggagagctacaggaatctctttggatgttgatgtgtgtttctctcaccctcggcttcgatggtcttgttcagagctgcataaactaacacatttatgtctccgagatctaagtgtggatcttctgtctgtgacacagtggccattgtagtttatcccgaagacgcctatgtacgtaagtttgcatttcctcccttctggtgatgactcagggttgtatagtatctgttaccccttccctcccagagtaaccataactcgttccgtttccaaacagccatggtggtgtccaattagctgtgtatcgctcttcccagagttgttaatgtggtgacatgcaccaacagccgtatgtgtactgtgatctgtaagaagtacaatgccatctgtctgccgaaggctagcatggttttaggtttatcttccttcacatccagaaattctgttggacactcacttccaccccaaactcctcaaatcaaaagccttcaaaacacgaggcactcttggatctaccctgagtatcctccaaactgtggatacagtttagtgagacaagcaatttctcccttctgagttattctctctgttggtggcaaaccacttcatagcaccaacagagatgtaggaaaaattcctcaaagtatttgtcatttctgagtcgcctgcattatcccattcttattctcctcaaacctgtgcatatatgacatgaaatgatatccattttttttttaagttagagacagagaggggaatacttatgcatggggagcctgttagcacagtgcctgccacaaaaacaagtgcccccgacaagatagttgctatgttatgacactttctcagatcaggattttctagtttaaaaattaaatatcataaaaccagcaaataatattaactaaaccttttatatagttctactctgttaattagtgtatttcatgtacatgaatatttttgaagtatttgggataactagccatcagtgtttgcagaagacctcagtctgttttgggccattacggaaacactaagctaatgtagactcgcctggccatttcctggtttcctcctgcattctgctttacattcttgaagaaagggataaagtggcaggttgaaaaattatcccatttcttctcctcatggtgtgcgttctgttttgtaagtattattttcttgcatgtgggcaagtgtcagggatgattcccttaaagctcaaggatgcatggaatagctccatctttgggaaagtacacctgacctacagtgttcctataatgaaaagtggttgcaataaatttcagggtcaatcatagggagacagagggaggcagtgggtgagaggagcttcctttcatttttgaaaggcagaaggggctgactaggtgctggggatgcatgtgcttgaaaatgacattttatttcccggatgctgtacctctgctgccccaagacccagccatggagaagcctggatcgaagggcccagaatagtcaccccaacatctgatcctaaatctgaatctccatgactgtgtagccttttagccctttctaccttctgcccttccagtgcattccagaccttaatccaccccagcaaggatcgcctccatttcctcatgagtctgggatgttttgacatgaatgaattaaaaagtcttcccagaatacagaagagtgatttgtcgtcaagccaaagaggctttctgtcttcctctggtgtcctctaaacccagggggctgccaaatgccacagagacctccagggagagtgagagttatacttggactgacttcatcggtcagaaagccttcccctaacagcagcattatttctgctattaaccccatacatctctggataaagaaactgaaaacgtggcctttggaagaatcaacagtaatgttgtgaaaaaaatatatattcttttcactgaccgaagtgttttttcaaataaataatcagaaatgttatgctacacaaagtttatggtatcccttctctactaattcaatgactcttcaatgtgatgatcccaagcaaaaaaaaaaaaaaaaaaaaaaaatcaactactgatcagatggtcaatttctaaggaaaagatgtagagttattcaaaggtcatctaagtcagttcagtctttgcaaaaagaatatcaggaatgtaacgtccagtggtaaatggaacccacagtatgagaagtaacaagaattaaataggaagcctggaagcctgagactatagatctatcacagaagattcccgtaacatatcccagctctgagttcttagatttgttgatgtcaaggagcccaaatttctgaacacaaatgcctgagtttcagctggcttaagtcagagctggcatccataatgatattttgttaattctaattggttttccccacttgagaaggacccaactagtcctcagataggtacacttgaatgcaaacggttgggtctctgtccatgggtaatagcacgcacatgcaaccaatttaaaatactctgggtaagagagtcttctagagcataaactgtacttgagaaacctttgtgcttctggaaaatgggtttcatcatccttacatctgcaggttcttaggccacttatggtttcaatgctctgagctggcaagtccctataggtctatctttctgccagagagacttgcaggattataatctcacaatctcaccactcattactgttatgtcctctgcaaatctctctgcgttattttagccagcaccatgactgggcatccacggattactcttgattttctcttccctgattcacaggatgccagagagtctgagtatttagtttttctggtgaaagcattaaaattcacacattgcagcaaaatctgtcaaatcatttcacgcttattttctggggagaaaaatagaaaattacaccttgtttacaaatgaaatagtttcacttaaatgggactctagccaaggcagttaattagtcaaaagatccacagctgtcatatagtcacacattgtttattctatttgctatgtcgaccactgatgtgtatttaatattctgcatccttcacaggaaatgacaaatgccctgaattatttgcaaatagaattgctaatgaaaaagaaaaaaaatgttgatgttaattaaatggtgattaaaaatttatctcctttaaatctaaaaagtgcagctttaataatcagttgtcatataaataagtaccttgtactgaaatcccttgcggtttaagacctaaataaacactaatttgagaactctaaataccttacagaggagctggatatattcgttaacattatgcacattcctgtactgtttctttgtgcatagctgttgtgcatgaatccttgaaaaggggacatgcgttgtttctgaaatggagcctgtttgtattaaccatggctatagtagacatgattggactcaaagaaaaagaagtttgagtaggtggaagctaaatgtgttgcttgatatttaatgagtgaagggttctatgtaaaggcaattttccttccaaaagtactgtttattttcactggagacaaaattagacctttccagataccccatctctttttggtgaacagggttgaaagtgctgtggggtctatacttgaaaagttctcccaatatgcagttactgtttcccattcaatccctatacattgtactgtgttcatatctaattgtatcagatgaggtcagcttgccacttgatatagatcagagatgatcaaatgcacttatcctgtgaatacaactgcttgtgtacacatgtatataatatacatgcttgatttctacaattcacacgtaagtgtattatccaaataaaattgtacatattgtaaagtttatgttaaaaattaaatggtgatgatggtattaacacatggaaaacccattctggttatttttatgggaggatatttctgtagagagggcactatatttttttaatctcagttgtagggaggatgacagagcctaagtagaatatgggtgttggcgttcttggccccttctttaaaaaacaaaactctgggatgaggggtatagaatatattctgctcgcatttgcttctgaagacaatgccgatatggacttgtgtgtgatttccttaaacacatcaaagggcatagatttcctatttaaattccttatcctctttcctcacctgcactctttctgtgcccagcagtactttcagtatagtattagctagaagtgcctgcccagaagggtcttagaaaaatagagttcgtagaaccacaccaaaagttaggagaaggggaggagagatgagaaaaatgaagtttacaaatgggcagttgcctgtttaagatcatcaagttagagagtatcagagcgaggattagatccaagacatggttcaatttctggctcgattctctgaccgggctgacttatttctaggtttctttcaaggggaaactcctgagtagctctggaataagagccagagatccccataaggctgccctggtttttgttttgttttgttttttgtttttgccacttgccccagaaagacctacccatgcttctttgaatttggttttcctttaaactccctcacaatccctgtcttaaagttcctagaagagaaaacttttctcagagaccgtctccttaggaaaaatcaacagggatattgtttaaaacctcaaactgaagataaaaggctggcagtgaccattgaaataattgaggtctatgtatcatgcattgaggaagaaccaagcacattgaccagctaataggacttcactgaatgtccactttattttgaaagccagtttgggattgtgggggaggggatgcaaaataaatagaaaatatagtctcttcccataagtagcttatggtttagaagttcagttctctaatacaaaggataaccaaggacccatgcaatgcaaacagagactaaacagcaaaaattagaaatgacaactaaagttgtagccaggaaaaaaaaaataatctgattctatgagcataccttttttaaaaaaaaaaaaaaagtccttccctaaacctttaagtaactccggagaatagagttagtggtcccggaaatgcagttgatttattttaatcttcaaataatggacttaggagacccaggagctgctaaccaggtgaacacactcacactcctacatttttgcacaggaaggcttttgtctggttccaaccagttctccagtccttgccaaatcatgcctcctcccagccttgcattatgacatgtgcctcaagaaaaggttgaaaaaattccccaaaggattagaaaatagaccatgatgtaaaaagacgaagcatttccctaaagcttttttgtttggttttgtttttgacatttgaagaacccttctgatacaccagggtatgaatgtatcagggaagcatagagggaaaattctttatcaagttccaagtgcctactgctgtggctgacagaaagcatcaggttccctagagagaaaacggggcaaagagatggcaaaaggaatagaggtgtatttcaggaaggatggcgaggtggagagggagcttttgtcactctgtctatcccattctgtgcattcttggaatgttacattttctgcttatcgcaagtacttgccatggatctgcacctgaatttgaaaatatcaaaatatatctaaattgagtgtaggcgatattctaggcatatcctagaatgtgatcctcaaaacaaccttaagatataaatatgattgctcccattttacagataaggaaactgaggctcaggtttttctcaacctcgtcagcaactagaattagctagtaaatccctaacgttagacatattttcaaagtgcctggtctcctccagggaggtcagaatagaagtcagaaagagaggtgtgctcttttgaggttcctgggcatatacccttgagtatcagcctctgtgacagggaagtctgttagggtcttacccctcattgcctgatctgtgtgtatggagaacttctggggatctcacctctgttcatagttccttctgagacttgaacactgtttgtgtctccctttcatatgcttgcacttagcatttaaaaaaatttctggggagttattttctctacttagcatgtgcctaatcttaaatatctctttcctaatgtctcatctttttatcagtcaacatcagggctaattccattaaacatatatacatagtacatgctctcatgtgtgtgggtatgcacacacacacacacagccatgtgaccctccattaaagtcctcctaaatattgtgacccatgttcaaagaggtgatattatttgcaagttgactattcctcctatttcatgtctatgtttggttatctttgccaggcagatggaatgagtttcagaggtgggacagggaataggaaatagagtttctgcatgaatgatggagagctttggtagtcagaatgattgaggaccagctattaaaatgttgttgttgttttccttcttggtctgttcctttttttttttcttttccaaattgtcttagagataattcccggctcacttagcattaggataatgacggttattctgtcccactgaagagtgttgaactggaacgcttccaccgggctttataggtacccagaatggcctaatgtttaacatatcctgcaggcatgcccctgagcacacactgcctttcctgcattcccccctctccatggtctaagccacatttctttcctggcatttatatttactgagaggtgaggggtgcattaggcccatttgatggatgaggacactagcggggatttgaaggaatgtctttctgggagtctccatgaattctctggagagttagctaacttaatgcaagttaagtgctagtctatgagaaaaatttctcagctgttttcatctttcaagctggcagtaaaaagccttttaagacagaaagattattatttgttatttggagatcctgttgttgggtctacaagtgcgtgaccatgttctccttggaatgttgagctatgcagaaatggagcactccaaagaacgatttgaccaatagcatttcttctctgggggttgtatttcaaagcatgcaactctccagggaaccagaactaaattgcttaaaatgaagtcattcctcagattaacttcctcagataaagtgtcagcggtctgcagaaacgaagaagacaaaactgagattatcactcataattctcttacttactatgtcagtgaaacaatgagtttgcatttttgcaatcctagaacattcttcattagccctgggtcatgacctcttccagttaattctctttcacacctttaggaaagatttaagatgaaccttcaataggatattaacataactcatagccaataccacagctgcctttcaaattaatgaggttaattgttctccagcaaacatgagtttgtctttggcattttaaatgcttcccattgatctgacattttgctgtttcaagttttaaagggctcaaatcaaagactattgataactgagcaaagagcgaagatccagaaatacgaaaacattgtctttttttttccatgaaaaacaatcatagccttttgaattcaatcgaagtttctacattagccatctaagacttatttaattatttctgttctcagtcaagctaattcaagtgaatgaacagtattgacttttaaaatcttttttaaatttttttaaatctttagtttattaagtttgtagaaaagctctggggccatgaccacttacgtaaatgtttcagtttaaaaacaaaagattcaggcctctaatttgagccaaatccaggtgatcttgtttgaaatttttgatgaatttgaaaagatgaaagtggaacttttaacattcatgttccccaaatttttcactgggaagggatgctaattgcctacttaagatataagttcaagaataacattttcatagaaaattcagaaaactgcttgacacagcagtgacatagttagatgtggctcagatgccttccaaacctgagggtccccaaagatttctttaccagttgtttttaactatgaatcttaatcttgttcattcccctgccaaaacaaatttaaaagcataaacctgctgaattaattggcagaattcaccatagtttatatcaccccagaatgtttatgtgtatatgtttatgtatagtatatatatttcaagctgaatgttcagaaagaataaggtgagtttcttggtagcttgaataagaattagatcataaaaattataaataatggccttctcaaagaggataaatgattcatttttcactcacgtattaataatatgacttgttgcataatacacaagctggtcattgcctgcaagaaagttattgaattaagcagctatccaggtttatgtctgagtccatttttaacccaacaaggctaggattgaactccatatgcagagggtttctattcacagctacattcaaacagaatccaaaacccacatcagaactgacttgatgtcttgtatggtggagctgaaagcaagctgtctgctctaagcaatggtcttcatttgggaattaatgttcactgatcaactgcagcagtttatatctgtcatgttttaatccactgcatgaggaagtgctaataagtgtattaaatatttccaagcatagctacaaaattatatgtttgcttaacacctttctatgatagaggaagtatatactatggttgtcttcttgtagtatgtcttttaattttgttcaatgtgttatcaactgttttaaggaatggggattgaaattcgtgtgcaaactgatatgtatataactcctgaggcaacagaagtgcccggtcatatggtgtaaaaatgtaaatacaggaaaatttcatttttctaataaagataatttctgccaattgatatgaaaatga

SEQ ID NO: 22 - DNA, 14006 bp, Homo sapiens
agccctgctgcagagcctccggctgggatagccgccccccgtgggggcgatgcggacagcgcgggacagccaggggagcgcgctggggccgcagcatgcgggaacccgctaaacccggtggctgctgaggcggccgagatgctcgtgcgcgcagcgcgccccactgcatcctcgaccttctcgggctacagggaccgtcagtggcgactatgggcagagtgggctattggaccctgctggtgctgccggcccttctggtctggcgcggtccggcgccgagcgcggcggcggagaagggtccccccgcgctaaatattgcggtgatgctgggtcacagccacgacgtgacagagcgcgaacttcgaacactgtggggccccgagcaggcggcggggctgcccctggacgtgaacgtggtagctctgctgatgaaccgcaccgaccccaagagcctcatcacgcacgtgtgcgacctcatgtccggggcacgcatccacggcctcgtgtttggggacgacacggaccaggaggccgtagcccagatgctggattttatctcctcccacaccttcgtccccatcttgggcattcatgggggcgcatctatgatcatggctgacaaggatccgacgtctaccttcttccagtttggagcgtccatccagcagcaagccacggtcatgctgaagatcatgcaggattatgactggcatgtcttctccctggtgaccactatcttccctggctacagggaattcatcagcttcgtcaagaccacagtggacaacagctttgtgggctgggacatgcagaatgtgatcacactggacacttcctttgaggatgcaaagacacaagtccagctgaagaagatccactcttctgtcatcttgctctactgttccaaagacgaggctgttctcattctgagtgaggcccgctcccttggcctcaccgggtatgatttcttctggattgtccccagcttggtctctgggaacacggagctcatcccaaaagagtttccatcgggactcatttctgtctcctacgatgactgggactacagcctggaggcgagagtgagggacggcattggcatcctaaccaccgctgcatcttctatgctggagaagttctcctacatccccgaggccaaggccagctgctacgggcagatggagaggccagaggtcccgatgcacaccttgcacccatttatggtcaatgttacatgggatggcaaagacttatccttcactgaggaaggctaccaggtgcaccccaggctggtggtgattgtgctgaacaaagaccgggaatgggaaaaggtgggcaagtgggagaaccatacgctgagcctgaggcacgccgtgtggcccaggtacaagtccttctccgactgtgagccggatgacaaccatctcagcatcgtcaccctggaggaggccccattcgtcatcgtggaagacatagaccccctgaccgagacgtgtgtgaggaacaccgtgccatgtcggaagttcgtcaaaatcaacaattcaaccaatgaggggatgaatgtgaagaaatgctgcaaggggttctgcattgatattctgaagaagctttccagaactgtgaagtttacttacgacctctatctggtgaccaatgggaagcatggcaagaaagttaacaatgtgtggaatggaatgatcggtgaagtggtctatcaacgggcagtcatggcagttggctcgctcaccatcaatgaggaacgttctgaagtggtggacttctctgtgccctttgtggaaacgggaatcagtgtcatggtttcaagaagtaatggcaccgtctcaccttctgcttttctagaaccattcagcgcctctgtctgggtgatgatgtttgtgatgctgctcattgtttctgccatagctgtttttgtctttgaatacttcagccctgttggatacaacagaaacttagccaaagggaaagcaccccatgggccttcttttacaattggaaaagctatatggcttctttggggcctggtgttcaataactccgtgcctgtccagaatcctaaagggaccaccagcaagatcatggtatctgtatgggccttcttcgctgtcatattcctggctagctacacagccaatctggctgccttcatgatccaagaggaatttgtggaccaagtgaccggcctcagtgacaaaaagtttcagagacctcatgactattccccaccttttcgatttgggacagtgcctaatggaagcacggagagaaacattcggaataactatccctacatgcatcagtacatgaccaaatttaatcagaaaggagtagaggacgccttggtcagcctgaaaacggggaagctggacgctttcatctacgatgccgcagtcttgaattacaaggctgggagggatgaaggctgcaagctggtgaccatcgggagtgggtacatctttgccaccaccggttatggaattgcccttcagaaaggctctccttggaagaggcagatcgacctggccttgcttcagtttgtgggtgatggtgagatggaggagctggagaccctgtggctcactgggatctgccacaacgagaagaacgaggtgatgagcagccagctggacattgacaacatggcgggcgtattctacatgctggctgccgccatggcccttagcctcatcaccttcatctgggagcacctcttctactggaagctgcgcttctgtttcacgggcgtgtgctccgaccggcctgggttgctcttctccatcagcaggggcatctacagctgcattcatggagtgcacattgaagaaaagaagaagtctccagacttcaatctgacgggatcccagagcaacatgttaaaactcctccggtcagccaaaaacatttccagcatgtccaacatgaactcctcaagaatggactcacccaaaagagctgctgacttcatccaaagaggttccctcatcatggacatggtttcagataaggggaatttgatgtactcagacaacaggtcctttcaggggaaagagagcatttttggagacaacatgaacgaactccaaacatttgtggccaaccggcagaaggataacctcaataactatgtattccagggacaacatcctcttactctcaatgagtccaaccctaacacggtggaggtggccgtgagcacagaatccaaagcgaactctagaccccggcagctgtggaagaaatccgtggattccatacgccaggattcactatcccagaatccagtctcccagagggatgaggcaacagcagagaataggacccactccctaaagagccctaggtatcttccagaagagatggcccactctgacatttcagaaacgtcaaatcgggccacgtgccacagggaacctgacaacagtaagaaccacaaaaccaaggacaactttaaaaggtcagtggcctccaaataccccaaggactgtagtgaggtcgagcgcacctacctgaaaaccaaatcaagctcccctagagacaagatctacactatagatggtgagaaggagcctggtttccacttagatccaccccagtttgttgaaaatgtgaccctgcccgagaacgtggacttcccggacccctaccaggatcccagtgaaaacttccgcaagggggactccacgctgccaatgaaccggaaccccttgcataatgaagaggggctttccaacaacgaccagtataaactctactccaagcacttcaccttgaaagacaagggttccccgcacagtgagaccagcgagcgataccggcagaactccacgcactgcagaagctgcctttccaacatgcccacctattcaggccacttcaccatgaggtcccccttcaagtgcgatgcctgcctgcggatggggaacctctatgacatcgatgaagaccagatgcttcaggagacagggatgaccaacgcttggttattgggagatgcccctcggacccttacaaacactcgttgccatcccaggcggtgaatgacagctatcttcggtcgtccttgaggtcaacggcatcgtactgttccagggacagtcggggccacaatgatgtgtatatttcggagcatgttatgccttatgctgcaaataagaataatatgtactctacccccagggttttaaattcctgcagcaatagacgcgtgtacaagaaaatgcctagtatcgaatctgatgtttaaaaatcttccattaatgttttatctatagggaaatatacgtaatggccaatgttctggagggtaaatgttggatgtccaatagtgccctgctaagaggaagaagatgtagggaggtattttgttgttgttgttgttggctcttttgcacacggcttcatgccataatcttccactcaaggaatcttgtgaggtgtgtgctgagcatggcagacaccagataggtgagtccttaaccaaaaataactaactacataagggcaagtctccgggacatgcctactgggtatgttggcaataatgatgcattggatgccaatggtgatgttatgatttcctatattccaaattccattaaggtcagcccaccatgtaattttctcatcagaaatgcctaatggtttctctaatacagaataagcaatatggtgtgcatgtaaacctgacacagacaaaataaaaacagttaagaatgcatctgcactgtagtgcgattgacatgtgcaagagattaggaagtttggctcgtaacagtttcagctttcttgttatgccttccatcacagcccaggctcaccccaagaactccaggctcccctaaagaatagcaaatcagtgtgttcgtgatgactgtgctaccttcattatagttcatttccaagacacatctggagccaaaggcccgagggaccctcaggtggggagagctacaggaatctctttggatgttgatgtgtgtttctctcaccctcggcttcgatggtcttgttcagagctgcataaactaacacatttatgtctccgagatctaagtgtggatcttctgtctgtgacacagtggccattgtagtttatcccgaagacgcctatgtacgtaagtttgcatttcctcccttctggtgatgactcagggttgtatagtatctgttaccccttccctcccagagtaaccataactcgttccgtttccaaacagccatggtggtgtccaattagctgtgtatcgctcttcccagagttgttaatgtggtgacatgcaccaacagccgtatgtgtactgtgatctgtaagaagtacaatgccatctgtctgccgaaggctagcatggttttaggtttatcttccttcacatccagaaattctgttggacactcacttccaccccaaactcctcaaatcaaaagccttcaaaacacgaggcactcttggatctaccctgagtatcctccaaactgtggatacagtttagtgagacaagcaatttctcccttctgagttattctctctgttggtggcaaaccacttcatagcaccaacagagatgtaggaaaaattcctcaaagtatttgtcatttctgagtcgcctgcattatcccattcttattctcctcaaacctgtgcatatatgacatgaaatgatatccattttttttttaagttagagacagagaggggaatacttatgcatggggagcctgttagcacagtgcctgccacaaaaacaagtgcccccgacaagatagttgctatgttatgacactttctcagatcaggattttctagtttaaaaattaaatatcataaaaccagcaaataatattaactaaaccttttatatagttctactctgttaattagtgtatttcatgtacatgaatatttttgaagtatttgggataactagccatcagtgtttgcagaagacctcagtctgttttgggccattacggaaacactaagctaatgtagactcgcctggccatttcctggtttcctcctgcattctgctttacattcttgaagaaagggataaagtggcaggttgaaaaattatcccatttcttctcctcatggtgtgcgttctgttttgtaagtattattttcttgcatgtgggcaagtgtcagggatgattcccttaaagctcaaggatgcatggaatagctccatctttgggaaagtacacctgacctacagtgttcctataatgaaaagtggttgcaataaatttcagggtcaatcatagggagacagagggaggcagtgggtgagaggagcttcctttcatttttgaaaggcagaaggggctgactaggtgctggggatgcatgtgcttgaaaatgacattttatttcccggatgctgtacctctgctgccccaagacccagccatggagaagcctggatcgaagggcccagaatagtcaccccaacatctgatcctaaatctgaatctccatgactgtgtagccttttagccctttctaccttctgcccttccagtgcattccagaccttaatccaccccagcaaggatcgcctccatttcctcatgagtctgggatgttttgacatgaatgaattaaaaagtcttcccagaatacagaagagtgatttgtcgtcaagccaaagaggctttctgtcttcctctggtgtcctctaaacccagggggctgccaaatgccacagagacctccagggagagtgagagttatacttggactgacttcatcggtcagaaagccttcccctaacagcagcattatttctgctattaaccccatacatctctggataaagaaactgaaaacgtggcctttggaagaatcaacagtaatgttgtgaaaaaaatatatattcttttcactgaccgaagtgttttttcaaataaataatcagaaatgttatgctacacaaagtttatggtatcccttctctactaattcaatgactcttcaatgtgatgatcccaagcaaaaaaaaaaaaaaaaaaaaaaaatcaactactgatcagatggtcaatttctaaggaaaagatgtagagttattcaaaggtcatctaagtcagttcagtctttgcaaaaagaatatcaggaatgtaacgtccagtggtaaatggaacccacagtatgagaagtaacaagaattaaataggaagcctggaagcctgagactatagatctatcacagaagattcccgtaacatatcccagctctgagttcttagatttgttgatgtcaaggagcccaaatttctgaacacaaatgcctgagtttcagctggcttaagtcagagctggcatccataatgatattttgttaattctaattggttttccccacttgagaaggacccaactagtcctcagataggtacacttgaatgcaaacggttgggtctctgtccatgggtaatagcacgcacatgcaaccaatttaaaatactctgggtaagagagtcttctagagcataaactgtacttgagaaacctttgtgcttctggaaaatgggtttcatcatccttacatctgcaggttcttaggccacttatggtttcaatgctctgagctggcaagtccctataggtctatctttctgccagagagacttgcaggattataatctcacaatctcaccactcattactgttatgtcctctgcaaatctctctgcgttattttagccagcaccatgactgggcatccacggattactcttgattttctcttccctgattcacaggatgccagagagtctgagtatttagtttttctggtgaaagcattaaaattcacacattgcagcaaaatctgtcaaatcatttcacgcttattttctggggagaaaaatagaaaattacaccttgtttacaaatgaaatagtttcacttaaatgggactctagccaaggcagttaattagtcaaaagatccacagctgtcatatagtcacacattgtttattctatttgctatgtcgaccactgatgtgtatttaatattctgcatccttcacaggaaatgacaaatgccctgaattatttgcaaatagaattgctaatgaaaaagaaaaaaaatgttgatgttaattaaatggtgattaaaaatttatctcctttaaatctaaaaagtgcagctttaataatcagttgtcatataaataagtaccttgtactgaaatcccttgcggtttaagacctaaataaacactaatttgagaactctaaataccttacagaggagctggatatattcgttaacattatgcacattcctgtactgtttctttgtgcatagctgttgtgcatgaatccttgaaaaggggacatgcgttgtttctgaaatggagcctgtttgtattaaccatggctatagtagacatgattggactcaaagaaaaagaagtttgagtaggtggaagctaaatgtgttgcttgatatttaatgagtgaagggttctatgtaaaggcaattttccttccaaaagtactgtttattttcactggagacaaaattagacctttccagataccccatctctttttggtgaacagggttgaaagtgctgtggggtctatacttgaaaagttctcccaatatgcagttactgtttcccattcaatccctatacattgtactgtgttcatatctaattgtatcagatgaggtcagcttgccacttgatatagatcagagatgatcaaatgcacttatcctgtgaatacaactgcttgtgtacacatgtatataatatacatgcttgatttctacaattcacacgtaagtgtattatccaaataaaattgtacatattgtaaagtttatgttaaaaattaaatggtgatgatggtattaacacatggaaaacccattctggttatttttatgggaggatatttctgtagagagggcactatatttttttaatctcagttgtagggaggatgacagagcctaagtagaatatgggtgttggcgttcttggccccttctttaaaaaacaaaactctgggatgaggggtatagaatatattctgctcgcatttgcttctgaagacaatgccgatatggacttgtgtgtgatttccttaaacacatcaaagggcatagatttcctatttaaattccttatcctctttcctcacctgcactctttctgtgcccagcagtactttcagtatagtattagctagaagtgcctgcccagaagggtcttagaaaaatagagttcgtagaaccacaccaaaagttaggagaaggggaggagagatgagaaaaatgaagtttacaaatgggcagttgcctgtttaagatcatcaagttagagagtatcagagcgaggattagatccaagacatggttcaatttctggctcgattctctgaccgggctgacttatttctaggtttctttcaaggggaaactcctgagtagctctggaataagagccagagatccccataaggctgccctggtttttgttttgttttgttttttgtttttgccacttgccccagaaagacctacccatgcttctttgaatttggttttcctttaaactccctcacaatccctgtcttaaagttcctagaagagaaaacttttctcagagaccgtctccttaggaaaaatcaacagggatattgtttaaaacctcaaactgaagataaaaggctggcagtgaccattgaaataattgaggtctatgtatcatgcattgaggaagaaccaagcacattgaccagctaataggacttcactgaatgtccactttattttgaaagccagtttgggattgtgggggaggggatgcaaaataaatagaaaatatagtctcttcccataagtagcttatggtttagaagttcagttctctaatacaaaggataaccaaggacccatgcaatgcaaacagagactaaacagcaaaaattagaaatgacaactaaagttgtagccaggaaaaaaaaaataatctgattctatgagcataccttttttaaaaaaaaaaaaaaagtccttccctaaacctttaagtaactccggagaatagagttagtggtcccggaaatgcagttgatttattttaatcttcaaataatggacttaggagacccaggagctgctaaccaggtgaacacactcacactcctacatttttgcacaggaaggcttttgtctggttccaaccagttctccagtccttgccaaatcatgcctcctcccagccttgcattatgacatgtgcctcaagaaaaggttgaaaaaattccccaaaggattagaaaatagaccatgatgtaaaaagacgaagcatttccctaaagcttttttgtttggttttgtttttgacatttgaagaacccttctgatacaccagggtatgaatgtatcagggaagcatagagggaaaattctttatcaagttccaagtgcctactgctgtggctgacagaaagcatcaggttccctagagagaaaacggggcaaagagatggcaaaaggaatagaggtgtatttcaggaaggatggcgaggtggagagggagcttttgtcactctgtctatcccattctgtgcattcttggaatgttacattttctgcttatcgcaagtacttgccatggatctgcacctgaatttgaaaatatcaaaatatatctaaattgagtgtaggcgatattctaggcatatcctagaatgtgatcctcaaaacaaccttaagatataaatatgattgctcccattttacagataaggaaactgaggctcaggtttttctcaacctcgtcagcaactagaattagctagtaaatccctaacgttagacatattttcaaagtgcctggtctcctccagggaggtcagaatagaagtcagaaagagaggtgtgctcttttgaggttcctgggcatatacccttgagtatcagcctctgtgacagggaagtctgttagggtcttacccctcattgcctgatctgtgtgtatggagaacttctggggatctcacctctgttcatagttccttctgagacttgaacactgtttgtgtctccctttcatatgcttgcacttagcatttaaaaaaatttctggggagttattttctctacttagcatgtgcctaatcttaaatatctctttcctaatgtctcatctttttatcagtcaacatcagggctaattccattaaacatatatacatagtacatgctctcatgtgtgtgggtatgcacacacacacacacagccatgtgaccctccattaaagtcctcctaaatattgtgacccatgttcaaagaggtgatattatttgcaagttgactattcctcctatttcatgtctatgtttggttatctttgccaggcagatggaatgagtttcagaggtgggacagggaataggaaatagagtttctgcatgaatgatggagagctttggtagtcagaatgattgaggaccagctattaaaatgttgttgttgttttccttcttggtctgttcctttttttttttcttttccaaattgtcttagagataattcccggctcacttagcattaggataatgacggttattctgtcccactgaagagtgttgaactggaacgcttccaccgggctttataggtacccagaatggcctaatgtttaacatatcctgcaggcatgcccctgagcacacactgcctttcctgcattcccccctctccatggtctaagccacatttctttcctggcatttatatttactgagaggtgaggggtgcattaggcccatttgatggatgaggacactagcggggatttgaaggaatgtctttctgggagtctccatgaattctctggagagttagctaacttaatgcaagttaagtgctagtctatgagaaaaatttctcagctgttttcatctttcaagctggcagtaaaaagccttttaagacagaaagattattatttgttatttggagatcctgttgttgggtctacaagtgcgtgaccatgttctccttggaatgttgagctatgcagaaatggagcactccaaagaacgatttgaccaatagcatttcttctctgggggttgtatttcaaagcatgcaactctccagggaaccagaactaaattgcttaaaatgaagtcattcctcagattaacttcctcagataaagtgtcagcggtctgcagaaacgaagaagacaaaactgagattatcactcataattctcttacttactatgtcagtgaaacaatgagtttgcatttttgcaatcctagaacattcttcattagccctgggtcatgacctcttccagttaattctctttcacacctttaggaaagatttaagatgaaccttcaataggatattaacataactcatagccaataccacagctgcctttcaaattaatgaggttaattgttctccagcaaacatgagtttgtctttggcattttaaatgcttcccattgatctgacattttgctgtttcaagttttaaagggctcaaatcaaagactattgataactgagcaaagagcgaagatccagaaatacgaaaacattgtctttttttttccatgaaaaacaatcatagccttttgaattcaatcgaagtttctacattagccatctaagacttatttaattatttctgttctcagtcaagctaattcaagtgaatgaacagtattgacttttaaaatcttttttaaatttttttaaatctttagtttattaagtttgtagaaaagctctggggccatgaccacttacgtaaatgtttcagtttaaaaacaaaagattcaggcctctaatttgagccaaatccaggtgatcttgtttgaaatttttgatgaatttgaaaagatgaaagtggaacttttaacattcatgttccccaaatttttcactgggaagggatgctaattgcctacttaagatataagttcaagaataacattttcatagaaaattcagaaaactgcttgacacagcagtgacatagttagatgtggctcagatgccttccaaacctgagggtccccaaagatttctttaccagttgtttttaactatgaatcttaatcttgttcattcccctgccaaaacaaatttaaaagcataaacctgctgaattaattggcagaattcaccatagtttatatcaccccagaatgtttatgtgtatatgtttatgtatagtatatatatttcaagctgaatgttcagaaagaataaggtgagtttcttggtagcttgaataagaattagatcataaaaattataaataatggccttctcaaagaggataaatgattcatttttcactcacgtattaataatatgacttgttgcataatacacaagctggtcattgcctgcaagaaagttattgaattaagcagctatccaggtttatgtctgagtccatttttaacccaacaaggctaggattgaactccatatgcagagggtttctattcacagctacattcaaacagaatccaaaacccacatcagaactgacttgatgtcttgtatggtggagctgaaagcaagctgtctgctctaagcaatggtcttcatttgggaattaatgttcactgatcaactgcagcagtttatatctgtcatgttttaatccactgcatgaggaagtgctaataagtgtattaaatatttccaagcatagctacaaaattatatgtttgcttaacacctttctatgatagaggaagtatatactatggttgtcttcttgtagtatgtcttttaattttgttcaatgtgttatcaactgttttaaggaatggggattgaaattcgtgtgcaaactgatatgtatataactcctgaggcaacagaagtgcccggtcatatggtgtaaaaatgtaaatacaggaaaatttcatttttctaataaagataatttctgccaattgatatgaaaatgagaaa

SEQ ID NO: 23 - DNA, 4271 bp, Homo sapiens
gcatcgcgagtgtccttgagccgcgggtgacggtggctctcgctgctcgcgccccctcctcccgcggggggagcctgatgccacgttccctatgaattatttatcgccggcctaaaaataccccgaacttcacagcccgagtgaccctccggtggacatgggtggggccctggggccggccctgttgctcacctcgctcttcggtgcctgggcagggctgggtccggggcagggcgagcagggcatgacggtggccgtggtgtttagcagctcagggccgccccaggcccagttccgtgcccgcctcaccccccagagcttcctggacctacccctggagatccagccgctcacagttggggtcaacaccaccaaccccagcagcctcctcacccagatctgcggcctcctgggtgctgcccacgtccacggcattgtctttgaggacaacgtggacaccgaggcggtggcccagatccttgacttcatctcctcccagacccatgtgcccatcctcagcatcagcggaggctctgctgtggtcctcacccccaaggagccgggctccgccttcctgcagctgggcgtgtccctggagcagcagctgcaggtgctgttcaaggtgctggaagagtacgactggagcgccttcgccgtcatcaccagcctgcacccgggccacgcgctcttcctggagggcgtgcgcgccgtcgccgacgccagccacgtgagttggcggctgctggacgtggtcacgctggagctgggcccgggagggccgcgcgcgcgcacgcagcgcctgctgcgccagctcgacgcgcccgtgtttgtggcctactgctcgcgcgaggaggccgaggtgctcttcgccgaggcggcgcaggccggtctggtggggcccggccacgtgtggctggtgcccaacctggcgctgggcagcaccgatgcgccccccgccaccttccccgtgggcctcatcagcgtcgtcaccgagagctggcgcctcagcctgcgccagaaggtgcgcgacggcgtggccattctggccctgggcgcccacagctactggcgccagcatggaaccctgccagccccggccggggactgccgtgttcaccctgggcccgtcagccctgcccgggaggccttctacaggcacctactgaatgtcacctgggagggccgagacttctccttcagccctggtgggtacctggtccagcccaccatggtggtgatcgccctcaaccggcaccgcctctgggagatggtggggcgctgggagcatggcgtcctatacatgaagtaccccgtgtggcctcgctacagtgcctctctgcagcctgtggtggacagtcggcacctgacggtggccacgctggaagagcggccctttgtcatcgtggagagccctgaccctggcacaggaggctgtgtccccaacaccgtgccctgccgcaggcagagcaaccacaccttcagcagcggggacgtggccccctacaccaagctctgctgtaagggattctgcatcgacatcctcaagaagctggccagagtggtcaaattctcctacgacctgtacctggtgaccaacggcaagcatggcaagcgggtgcgcggcgtatggaacggcatgattggggaggtgtactacaagcgggcagacatggccatcggctccctcaccatcaatgaggaacgctccgagatcgtagacttctctgtaccctttgtggagacgggcatcagtgtgatggtggctcgcagcaatggcaccgtctccccctcggccttcttggagccatatagccctgcagtgtgggtgatgatgtttgtcatgtgcctcactgtggtggccatcaccgtcttcatgttcgagtacttcagccctgtcagctacaaccagaacctcaccagaggcaagaagtccgggggcccagctttcactatcggcaagtccgtgtggctgctgtgggcgctggtcttcaacaactcagtgcccatcgagaacccgcggggcaccaccagcaagatcatggttctggtctgggccttctttgctgtcatcttcctcgccagctacacggccaacctggccgccttcatgatccaagagcaatacatcgacactgtgtcgggcctcagtgacaagaagtttcagcggcctcaagatcagtacccacctttccgcttcggcacggtgcccaacggcagcacggagcggaacatccgcagtaactaccgtgacatgcacacccacatggtcaagttcaaccagcgctcggtggaggacgcgctcaccagcctcaagatggggaagctggatgccttcatctatgatgctgctgtcctcaactacatggcaggcaaggacgagggctgcaagctggtcaccattgggtctggcaaggtctttgctaccactggctacggcatcgccatgcagaaggactcccactggaagcgggccatagacctggcgctcttgcagttcctgggggacggagagacacagaaactggagacagtgtggctctcagggatctgccagaatgagaagaacgaggtgatgagcagcaagctggacatcgacaacatggcaggcgtcttctacatgctgctggtggccatggggctggccctgctggtcttcgcctgggagcacctggtctactggaagctgcgccactcggtgcccaactcatcccagctggacttcctgctggctttcagcaggggcatctacagctgcttcagcggggtgcagagcctcgccagcccaccgcggcaggccagcccggacctcacggccagctcggcccaggccagcgtgctcaagatgctgcaggcagcccgcgacatggtgaccacggcgggcgtaagcagctccctggaccgcgccactcgcaccatcgagaattggggtggcggccgccgtgcgcccccaccgtccccctgcccgaccccgcggtctggccccagcccatgcctgcccacccccgacccgcccccagagccgagccccacgggctggggaccgccagacgggggtcgcgcggcgcttgtgcgcagggctccgcagcccccgggccgccccccgacgccggggccgcccctgtccgacgtctcccgagtgtcgcgccgcccagcctgggaggcgcggtggccggtgcggaccgggcactgcgggaggcacctctcggcctccgagcggcccctgtcgcccgcgcgctgtcactacagctcctttcctcgagccgaccgatccggccgccccttcctcccgctcttcccggagctggaggacctgccgctgctcggtccggagcagctggcccggcgggaggccctgctgcacgcggcctgggcccggggctcgcgcccgcgtcacgcttccctgcccagctccgtggccgaggccttcgctcggcccagctcgctgcccgctgggtgcaccggccccgcctgcgcccgccccgacggccactcggcctgcaggcgcttggcgcaggcgcagtcgatgtgcttgccgatctaccgggaggcctgccaggagggcgagcaggcaggggcccccgcctggcagcacagacagcacgtctgcctgcacgcccacgcccacctgccattttgctggggggctgtctgtcctcaccttccaccctgtgccagccacggctcctggctctccggggcctgggggcctctggggcacaggggcaggactctggggctgggcacaggctacagagacagtgggggactggacgagatcagcagggtagcccgtgggacgcaaggcttcccgggaccctgcacctggagacggatctccagtctggagtcagaagtgtgagttatcagccactcaggctccgagccagctggattctctgcctgccactgtcagggttaagcggcaggcaggattgggcttttctggcttctgccatgaaatcctggccatgggaccccagtgacagatgatgtcttccatggtcatcagtgacctcagtagcctcaaatcatggtgagggctgggcttttgctgtcctcttctcacgcagagttctgccaggagggtgtgctgtgggggtcagactcctgaggctctcccttccctggggctagccagttactggtcatggctgctgtgggcatggaggctggaacttgtggttgaggcagggccatcccgatccttgctctacctggctagagtttcttctcatcagagcactgggacattaaaccaaccttttacaaca

SEQ ID NO: 24 - DNA, 3240 bp, Homo sapiens
gcgagtcccgcttcttccccttccagctctgccccatctcctttgccccagcctagctctctccttcctgcgacctgggagaatcggagtgggcttccagagtagtggcatcttgcagatgctggcccgtgcccggctcccgggcttccagagcgcaaagggaagggtaggcagagagggcactgggcgagcaagacctggcgctgggccctggctgcgcaccagccgcctgcgccggtctctgggggcttcgcccaggatccccgacggcggggaggggaagccccgagtctcctgtttcctgcctacgcgggcagggcggggacacgcggagcaaaggcacccggcaacctgaacccccaggtgcgtcccgcgaccctgcaaggagctgctgggaccctccggtggacatgggtggggccctggggccggccctgttgctcacctcgctcttcggtgcctgggcagggctgggtccggggcagggcgagcagggcatgacggtggccgtggtgtttagcagctcagggccgccccaggcccagttccgtgcccgcctcaccccccagagcttcctggacctacccctggagatccagccgctcacagttggggtcaacaccaccaaccccagcagcctcctcacccagatctgcggcctcctgggtgctgcccacgtccacggcattgtctttgaggacaacgtggacaccgaggcggtggcccagatccttgacttcatctcctcccagacccatgtgcccatcctcagcatcagcggaggctctgctgtggtcctcacccccaaggagccgggctccgccttcctgcagctgggcgtgtccctggagcagcagctgcaggtgctgttcaaggtgctggaagagtacgactggagcgccttcgccgtcatcaccagcctgcacccgggccacgcgctcttcctggagggcgtgcgcgccgtcgccgacgccagccacgtgagttggcggctgctggacgtggtcacgctggagctgggcccgggagggccgcgcgcgcgcacgcagcgcctgctgcgccagctcgacgcgcccgtgtttgtggcctactgctcgcgcgaggaggccgaggtgctcttcgccgaggcggcgcaggccggtctggtggggcccggccacgtgtggctggtgcccaacctggcgctgggcagcaccgatgcgccccccgccaccttccccgtgggcctcatcagcgtcgtcaccgagagctggcgcctcagcctgcgccagaaggtgcgcgacggcgtggccattctggccctgggcgcccacagctactggcgccagcatggaaccctgccagccccggccggggactgccgtgttcaccctgggcccgtcagccctgcccgggaggccttctacaggcacctactgaatgtcacctgggagggccgagacttctccttcagccctggtgggtacctggtccagcccaccatggtggtgatcgccctcaaccggcaccgcctctgggagatggtggggcgctgggagcatggcgtcctatacatgaagtaccccgtgtggcctcgctacagtgcctctctgcagcctgtggtggacagtcggcacctgacggtggccacgctggaagagcggccctttgtcatcgtggagagccctgaccctggcacaggaggctgtgtccccaacaccgtgccctgccgcaggcagagcaaccacaccttcagcagcggggacgtggccccctacaccaagctctgctgtaagggattctgcatcgacatcctcaagaagctggccagagtggtcaaattctcctacgacctgtacctggtgaccaacggcaagcatggcaagcgggtgcgcggcgtatggaacggcatgattggggaggtgtactacaagcgggcagacatggccatcggctccctcaccatcaatgaggaacgctccgagatcgtagacttctctgtaccctttgtggagacgggcatcagtgtgatggtggctcgcagcaatggcaccgtctccccctcggccttcttggagccatatagccctgcagtgtgggtgatgatgtttgtcatgtgcctcactgtggtggccatcaccgtcttcatgttcgagtacttcagccctgtcagctacaaccagaacctcaccagaggcaagaagtccgggggcccagctttcactatcggcaagtccgtgtggctgctgtgggcgctggtcttcaacaactcagtgcccatcgagaacccgcggggcaccaccagcaagatcatggttctggtctgggccttctttgctgtcatcttcctcgccagctacacggccaacctggccgccttcatgatccaagagcaatacatcgacactgtgtcgggcctcagtgacaagaagtttcagcggcctcaagatcagtacccacctttccgcttcggcacggtgcccaacggcagcacggagcggaacatccgcagtaactaccgtgacatgcacacccacatggtcaagttcaaccagcgctcggtggaggacgcgctcaccagcctcaagatggggaagctggatgccttcatctatgatgctgctgtcctcaactacatggcaggcaaggacgagggctgcaagctggtcaccattgggtctggcaaggtctttgctaccactggctacggcatcgccatgcagaaggactcccactggaagcgggccatagacctggcgctcttgcagttcctgggggacggagagacacagaaactggagacagtgtggctctcagggatctgccagaatgagaagaacgaggtgatgagcagcaagctggacatcgacaacatggcaggcgtcttctacatgctgctggtggccatggggctggccctgctggtcttcgcctgggagcacctggtctactggaagctgcgccactcggtgcccaactcatcccagctggacttcctgctggctttcagcagggtgggtgcccacccctccccacacaggccaaagttttaagggcacctacccagataagtccgggctggccatggcccatttccagaggtaaaaccaaggttctggcccggctcatggctcacacctgtaatctcaacactttgagaggcttaggccagtggatcccttgagtccagaagtttgagaccagcctgggcaacatggcgaaaacccatctctacagaaaataaaagaattagctgggaaa

SEQ ID NO: 25 - DNA, 5511 bp, Homo sapiens
atcccgagtgagtgtgtgtgtgcgagaacacagcgagtgtgtgagtccctcccgctccagctcctccaagccgcggccgccgccgccaccctcgcccgcagcctcccgcagcctccctcggccaccggtgcctgctgggggtgttgcctgggtaggtcggcccggcccccaggggtctctcgagcgtctgccatctgcccgagcaacatgtgtagccacgtcctcgcctagtccaggtggccgcaaccttgggggagagacagggcaggacaggaccaaggaagaggaaggagagacggagccagggacagacaggaggtccgggctgccgctgctgccgccaccaccactgccgccgccccggggcctgccccccgacatcggctctctgagccctcctcggaatcttggggtcgctggacgccgggttccggtcctggcccccccgccatccccccaacagaacagggtcatgaaaagaggccgcccggcggggcccgcaggcgatgcgcggcgccggtggcccccgcggccctcggggccccgctaagatgctgctgctgctggcgctggcctgcgccagcccgttcccggaggaggcgccggggccgggcggggccggtgggcccggcggcggcctcggcggggcgcggccgctcaacgtggcgctcgtgttctcggggcccgcgtacgcggccgaggcggcacgcctgggcccggccgtggcggcggcggtgcgcagcccgggcctagacgtgcggcccgtggcgctggtgctcaacggctcggacccgcgcagcctcgtgctgcagctctgcgacctgctgtcggggttgcgcgtgcacggcgtggtcttcgaagacgactcgcgcgcgcccgccgtcgcgcccatcctcgacttcctgtcggcgcagacctcgctgcccatcgtggccgtgcacggcggcgccgcgctcgtgctcacgcccaaggagaagggctccaccttcctgcagctgggctcttccaccgagcaacagcttcaggtcatctttgaggtgctggaggagtatgactggacgtcctttgtagccgtgaccactcgtgcccctggccaccgggccttcctgtcctacattgaggtgctgactgacggtagtctggtgggctgggagcaccgcggagcgctgacgctggaccctggggcgggcgaggccgtgctcagtgcccagctccgcagtgtcagcgcgcagatccgcctgctcttctgcgcccgagaggaggccgagcccgtgttccgcgcagctgaggaggctggcctcactggatctggctacgtctggttcatggtggggccccagctggctggaggcgggggctctggggcccctggtgagccccctcttctgccaggaggcgcccccctgcctgccgggctgtttgcagtgcgctcggctggctggcgggatgacctggctcggcgagtggcagctggcgtggccgtagtggccagaggtgcccaggccctgctgcgtgattatggtttccttcctgagctcggccacgactgtcgcgcccagaaccgcacccaccgcggcgagagtctgcataggtacttcatgaacatcacgtgggataaccgggattactccttcaatgaggacggcttcctagtgaacccctccctggtggtcatctccctcaccagagacaggacgtgggaggtggtgggcagctgggagcagcagacgctccgcctcaagtacccgctgtggtcccgctatggtcgcttcctgcagccagtggacgacacgcagcacctcacggtggccacgctggaggaaaggccgtttgtcatcgtggagcctgcagaccctatcagcggcacctgcatccgagactccgtcccctgccggagccagctcaaccgaacccacagccctccaccggatgccccccgcccggaaaagcgctgctgcaagggtttctgcatcgacattctgaagcggctggcgcataccatcggcttcagctacgacctctacctggtcaccaatggcaagcacggaaagaagatcgatggcgtctggaacggcatgatcggggaggtgttctaccagcgcgcagacatggccatcggctccctcaccatcaacgaggagcgctccgagatcgtggacttctccgtccccttcgtggagaccggcatcagcgtcatggtggcgcgcagcaatggcacggtgtccccctcggccttcctcgagccctacagccccgccgtgtgggtgatgatgttcgtcatgtgcctcactgtggtcgccgtcactgttttcatcttcgagtacctcagtcctgttggttacaaccgcagcctggccacgggcaagcgccctggcggttcaaccttcaccattgggaaatccatctggctgctctgggccctggtgttcaataattcggtgcccgtggagaacccccggggaaccaccagcaaaatcatggtgctggtgtgggccttcttcgccgtcatcttcctcgccagctacacagccaacctggccgccttcatgatccaggaggagtacgtggatactgtgtctgggctcagtgaccgcaagttccagaggccccaggagcagtacccgcccctgaagtttgggaccgtgcccaacggctccacggagaagaacatccgcagcaactatcccgacatgcacagctacatggtgcgctacaaccagccccgcgtagaggaagcgctcactcagctcaaggcagggaagctggacgccttcatctacgatgctgcagtgctcaattacatggcccgcaaggacgagggctgcaagcttgtcaccatcggctccggcaaggtcttcgccacgacaggctatggcatcgccctgcacaagggctcccgctggaagcggcccatcgacctggcgttgctgcagttcctgggggatgatgagatcgagatgctggagcggctgtggctctctgggatctgccacaatgacaaaatcgaggtgatgagcagcaagctggacatcgacaacatggcgggcgtcttctacatgctcctggtggccatgggcctgtccctgctggtcttcgcctgggagcacctggtgtactggcgcctgcggcactgcctggggcccacccaccgcatggacttcctgctggccttctccaggggcatgtacagctgctgcagcgctgaggccgccccaccgcccgccaagcccccgccgccgccacagcccctgcccagccccgcgtaccccgcgccgcggccggctcccgggcccgcacctttcgtgccccgcgagcgcgcctcagtggaccgctggcgccggaccaagggcgcggggccgccggggggcgcgggcctggccgacggcttccaccgctactacggccccatcgagccgcagggcctaggcctcggcctgggcgaagcgcgcgcggcaccgcggggcgcagccgggcgcccgctgtccccgccggccgctcagcccccgcagaagccgccgccctcctatttcgccatcgtacgcgacaaggagccagccgagccccccgccggcgccttccccggcttcccgtcgccgcccgcgccccccgccgccgcggccaccgccgtcgggccgccactctgccgcttggccttcgaggacgagagcccgccggcgcccgcgcggtggccgcgctcggaccccgagagccaacccctgctggggccaggcgcgggcggcgcggggggcacggggggcgcaggcggaggagccccggccgctccgcccccgtgccgcgccgcgccgcccccgtgcccttacctcgatctcgagccgtcgccgtcggactcggaggactcggagagcctgggcggcgcgtcgctgggcggcctggagccctggtggttcgccgacttcccttacccgtatgccgagcgcctcgggccgccgcccggccgctactggtcggtcgacaagctcgggggctggcgcgccgggagctgggactacctgcccccgcgcagcggtccggccgcctggcactgtcggcactgcgccagcctggagctgctgccgccgccgcgccatctcagctgctcgcacgatggcctggacggcggctggtgggcgccaccgcctccaccctgggccgccgggcccctgccccgacgccgggcccgctgcgggtgcccgcggtcgcacccgcaccgcccgcgggcctcgcaccgcacgcccgccgccgccgcgccccaccaccacaggcaccggcgcgccgctgggggctgggacctcccgccgcccgcgcccacctcgcgctcgctcgaggacctcagctcgtgccctcgcgccgcccctgcgcgcaggcttaccgggccctcccgccacgctcgcaggtgtccgcacgccgcgcactgggggccgccgctgcccacagcttcccaccggagacaccggggcggggacctgggcacccgcaggggctcggcgcacttctctagcctcgagtccgaggtatgacgcggccccgggggccccaccgcccccttggtcagcgcaggccacggcccgagggggcgcccgcagtggacaggacccgcgtgggttgggaaggaaagcagtggaactggccggaccccgcctggagcagcgtcctgcgccccctggttctggaggaaccgcaagccggagaggatttggtccctcaactatcacccagcctgagaacgagggggcgggggccttggagcccaccggacttttttttaaacccgacaagggctttttaacgtcaccagatggggcgggaggtggggggttcacgccactcccgcgtcccacctccacgcccggggccgtggcccccacatcactgtgcagctccccggccccagccgcgcctccggggaagcccgtttttaacctttcatccattgggactcaaaactgtgagacgcgttcgccaaactgtgaccccagaccttggccccgccacacagaaacccctgacccagactgtgacatccgactcctaaaatggtcatccgactccagactgtgacccctgaccccaaactgtgacccgaaccccagactgtgccccgaccagactgtgacccttgacatcaaaccgtgacaccccctcctcctcttccaccctcggtcgcttttccctactctgacctaggacacgtccccaacggaagccccgccttcccttgcaccgcgatgaacccaacctccctgaagccaaaacttcccaccctgccgcacctccggaccaccccacttgccaccaagcacatcctctccaaatccaacccttatttgcgacccttcagtgatgacccagcagacctccaaaaagcctcctctgcccagatctccagccgggtctggggctgggttgcggaggggagggtctgggagtccacacttctccaccaacttcccttcccactctcttattccacattgcagtgtttggaataaaccatgtttttatgcctcctca